SEQUENCE LISTING (205 sequences)

SEQ ID NO: 1 - protein, 327 aa, synthetic construct
  REGION 1..327: note = artificially synthesized sequence
MSHLAELVASAKAAISQASDVAALDNVRVEYLGKKGHLTLQMTTLRELPPEERPAAGAVINEAKEQVQQALNARKAELESAALNARLAAETIDVSLPGRRIENGGLHPVTRTIDRIESFFGELGFTVATGPEIEDDYHNFDALNIPGHHPARADHDTFWFDTTRLLRTATSGVQIRTMKAQQPPIRIIAPGRVYRNDYDQTHTPMFHQMEGLIVDTNISFTNLKGTLHDFLRNFFEEDLQIRFRPSYFPFTEPSAEVDVMGKNGKWLEVLGCGMVHPNVLRNVGIDPEVYSGFAFGMGMERLTMLRYGVTDLRSFFENDLRFLKQFK

SEQ ID NO: 2 - protein, 327 aa, synthetic construct
  REGION 1..327: note = artificially synthesized sequence
MSHLAELVASAKAAISQASDVAALDNVRVEYLGKKGHLTLQMTTLRELPPEERPAAGAVINEAKEQVQQALNARKAELESAALNARLAAETIDVSLPGRRIENGGLHPVTRTIDRIESFFGELGFTVATGPEIEDDYHNFDALNIPGHHPARADHDTFWFDTTRLLRTGTSGVQIRTMKAQQPPIRIIAPGRVYRNDYDQTHTPMFHQMEGLIVDTNISFTNLKGTLHDFLRNFFEEDLQIRFRPSYFPFTEPSAEVDVMGKNGKWLEVLGCGMVHPNVLRNVGIDPEVYSGFAFGMGMERLTMLRYGVTDLRSFFENDLRFLKQFK

SEQ ID NO: 3 - protein, 951 aa, synthetic construct
  REGION 1..951: note = artificially synthesized sequence
MEKTYNPQDIEQPLYEHWEKQGYFKPNGDESQESFCIMIPPPAVTGSLHMGHAFQQTIMDTMIRYQRMQGKNTLWQVGTDHAGIATQMVVERKIAAEEGKTRHDYGREAFIDKIWEWKAESGGTITRQMRRLGNSVDWERERFTMDEGLSNAVKEVFVRLYKEDLIYRGKRLVNWDPKLRTAISDLEVENRESKGSMWHIRYPLADGAKTADGKDYLVVATTRPETLLGDTGVAVNPEDPRYKDLIGKYVILPLVNRRIPIVGDEHADMEKGTGCVKITPAHDFNDYEVGKRHALPMINILTFDGDIRESAQVFDTKGNESDVYSSEIPAEFQKLERFAARKAVVAAVDALGLLEEIKPHDLTVPYGDRGGVVIEPMLTDQWYVRADVLAKPAVEAVENGDIQFVPKQYENMYFSWMRDIQDWCISRQLWWGHRIPAWYDEAGNVYVGRNEDEVRKENNLGADVVLRQDEDVLDTWFSSALWTFSTLGWPENTDALRQFHPTSVMVSGFDIIFFWIARMIMMTMHFIKDENGKPQVPFHTVYMTGLIRDDEGQKMSKSKGNVIDPLDMVDGISLPELLEKRTGNMMQPQLADKIRKRTEKQFPNGIEPHGTDALRFTLAALASTGRDINWDMKRLEGYRNFCNKLWNASRFVLMNTEGQDCGFNGGEMTLSLADRWILAEFNQTIKAYREALDSFRFDIAAGILYEFTWNQFCDWYLELTKPVMNGGTEAELRGTRHTLVTVLEGLLRLAHPIIPFITETIWQRVKVLCGITADTIMLQPFPQYDASQVDEAALADTEWLKQAIVAVRNIRAEMNIAPGKPLELLLRGCSADAERRVNENRGFLQTLARLESITVLPADDKGPVSVTKIIDGAELLIPMAGLINKEDELARLAKEVAKIEGEISRIENKLANEGFVARAPEAVIAKEREKLEGYAEAKAKLIEQQAVIAAL

SEQ ID NO: 4 - protein, 951 aa, synthetic construct
  REGION 1..951: note = artificially synthesized sequence
MEKTYNPQDIEQPLYEHWEKQGYFKPNGDESQESFCIMIPPPGVTGSLHMGHAFQQTIMDTMIRYQRMQGKNTLWQVGTDHAGIATQMVVERKIAAEEGKTRHDYGREAFIDKIWEWKAESGGTITRQMRRLGNSVDWERERFTMDEGLSNAVKEVFVRLYKEDLIYRGKRLVNWDPKLRTAISDLEVENRESKGSMWHIRYPLADGAKTADGKDYLVVATTRPETLLGDTGVAVNPEDPRYKDLIGKYVILPLVNRRIPIVGDEHADMEKGTGCVKITPAHDFNDYEVGKRHALPMINILTFDGDIRESAQVFDTKGNESDVYSSEIPAEFQKLERFAARKAVVAAVDALGLLEEIKPHDLTVPYGDRGGVVIEPMLTDQWYVRADVLAKPAVEAVENGDIQFVPKQYENMYFSWMRDIQDWCISRQLWWGHRIPAWYDEAGNVYVGRNEDEVRKENNLGADVVLRQDEDVLDTWFSSALWTFSTLGWPENTDALRQFHPTSVMVSGFDIIFFWIARMIMMTMHFIKDENGKPQVPFHTVYMTGLIRDDEGQKMSKSKGNVIDPLDMVDGISLPELLEKRTGNMMQPQLADKIRKRTEKQFPNGIEPHGTDALRFTLAALASTGRDINWDMKRLEGYRNFCNKLWNASRFVLMNTEGQDCGFNGGEMTLSLADRWILAEFNQTIKAYREALDSFRFDIAAGILYEFTWNQFCDWYLELTKPVMNGGTEAELRGTRHTLVTVLEGLLRLAHPIIPFITETIWQRVKVLCGITADTIMLQPFPQYDASQVDEAALADTEWLKQAIVAVRNIRAEMNIAPGKPLELLLRGCSADAERRVNENRGFLQTLARLESITVLPADDKGPVSVTKIIDGAELLIPMAGLINKEDELARLAKEVAKIEGEISRIENKLANEGFVARAPEAVIAKEREKLEGYAEAKAKLIEQQAVIAAL

SEQ ID NO: 5 - protein, 951 aa, synthetic construct
  REGION 1..951: note = artificially synthesized sequence
MEKTYNPQDIEQPLYEHWEKQGYFKPNGDESQESFCIMIPPPGVSGSLHMGHAFQQTIMDTMIRYQRMQGKNTLWQVGTDHAGIATQMVVERKIAAEEGKTRHDYGREAFIDKIWEWKAESGGTITRQMRRLGNSVDWERERFTMDEGLSNAVKEVFVRLYKEDLIYRGKRLVNWDPKLRTAISDLEVENRESKGSMWHIRYPLADGAKTADGKDYLVVATTRPETLLGDTGVAVNPEDPRYKDLIGKYVILPLVNRRIPIVGDEHADMEKGTGCVKITPAHDFNDYEVGKRHALPMINILTFDGDIRESAQVFDTKGNESDVYSSEIPAEFQKLERFAARKAVVAAVDALGLLEEIKPHDLTVPYGDRGGVVIEPMLTDQWYVRADVLAKPAVEAVENGDIQFVPKQYENMYFSWMRDIQDWCISRQLWWGHRIPAWYDEAGNVYVGRNEDEVRKENNLGADVVLRQDEDVLDTWFSSALWTFSTLGWPENTDALRQFHPTSVMVSGFDIIFFWIARMIMMTMHFIKDENGKPQVPFHTVYMTGLIRDDEGQKMSKSKGNVIDPLDMVDGISLPELLEKRTGNMMQPQLADKIRKRTEKQFPNGIEPHGTDALRFTLAALASTGRDINWDMKRLEGYRNFCNKLWNASRFVLMNTEGQDCGFNGGEMTLSLADRWILAEFNQTIKAYREALDSFRFDIAAGILYEFTWNQFCDWYLELTKPVMNGGTEAELRGTRHTLVTVLEGLLRLAHPIIPFITETIWQRVKVLCGITADTIMLQPFPQYDASQVDEAALADTEWLKQAIVAVRNIRAEMNIAPGKPLELLLRGCSADAERRVNENRGFLQTLARLESITVLPADDKGPVSVTKIIDGAELLIPMAGLINKEDELARLAKEVAKIEGEISRIENKLANEGFVARAPEAVIAKEREKLEGYAEAKAKLIEQQAVIAAL

SEQ ID NO: 6 - protein, 430 aa, synthetic construct
  REGION 1..430: note = artificially synthesized sequence
MLDPNLLRNEPDAVAEKLARRGFKLDVDKLGALEERRKVLQVKTENLQAERNSRSKSIGQAKARGEDIEPLRLEVNKLGEELDAAKAELDALQAEIRDIALTIPNLPADEVPVGKDENDNVEVSRWGTPREFDFEVRDHVTLGEMHSGLDFAAAVKLTGSRFVVMKGQIARMHRALSQFMLDLHTEQHGYSENYVPYLVNQDTLYGTGQLPKFAGDLFHTRPLEEEADTSNYALIPSAEVPLTNLVRGEIIDEDDLPIKMTAHTPCFRSEAGSYGRDTRGLIRMHQFDKVEMVQIVRPEDSMAALEEMTGHAEKVLQLLGLPYRKIILCTGDMGFGACKTYDLEVWIPAQNTYREISSCSNVWDFQARRMQARCRSKSDKKTRLVHTLNGSGLAVGRTLVAVMENYQQADGRIEVPEVLRPYMNGLEYIG

SEQ ID NO: 7 - protein, 430 aa, synthetic construct
  REGION 1..430: note = artificially synthesized sequence
MLDPNLLRNEPDAVAEKLARRGFKLDVDKLGALEERRKVLQVKTENLQAERNSRSKSIGQAKARGEDIEPLRLEVNKLGEELDAAKAELDALQAEIRDIALTIPNLPADEVPVGKDENDNVEVSRWGTPREFDFEVRDHVTLGEMHSGLDFAAAVKLTGSRFVVMKGQIARMHRALSQFMLDLHTEQHGYSENYVPYLVNQDTLYGTGQLPKFAGDLFHTRPLEEEADTSNYALIPTAAVPLTNLVRGEIIDEDDLPIKMTAHTPCFRSEAGSYGRDTRGLIRMHQFDKVEMVQIVRPEDSMAALEEMTGHAEKVLQLLGLPYRKIILCTGDMGFGACKTYDLEVWIPAQNTYREISSCSNVWDFQARRMQARCRSKSDKKTRLVHTLNGSGLAVGRTLVAVMENYQQADGRIEVPEVLRPYMNGLEYIG

SEQ ID NO: 8 - protein, 430 aa, synthetic construct
  REGION 1..430: note = artificially synthesized sequence
MLDPNLLRNEPDAVAEKLARRGFKLDVDKLGALEERRKVLQVKTENLQAERNSRSKSIGQAKARGEDIEPLRLEVNKLGEELDAAKAELDALQAEIRDIALTIPNLPADEVPVGKDENDNVEVSRWGTPREFDFEVRDHVTLGEMHSGLDFAAAVKLTGSRFVVMKGQIARMHRALSQFMLDLHTEQHGYSENYVPYLVNQDTLYGTGQLPKFAGDLFHTRPLEEEADTSNYALIPTAGVPLTNLVRGEIIDEDDLPIKMTAHTPCFRSEAGSYGRDTRGLIRMHQFDKVEMVQIVRPEDSMAALEEMTGHAEKVLQLLGLPYRKIILCTGDMGFGACKTYDLEVWIPAQNTYREISSCSNVWDFQARRMQARCRSKSDKKTRLVHTLNGSGLAVGRTLVAVMENYQQADGRIEVPEVLRPYMNGLEYIG

SEQ ID NO: 9 - protein, 430 aa, synthetic construct
  REGION 1..430: note = artificially synthesized sequence
MLDPNLLRNEPDAVAEKLARRGFKLDVDKLGALEERRKVLQVKTENLQAERNSRSKSIGQAKARGEDIEPLRLEVNKLGEELDAAKAELDALQAEIRDIALTIPNLPADEVPVGKDENDNVEVSRWGTPREFDFEVRDHVTLGEMHSGLDFAAAVKLTGSRFVVMKGQIARMHRALSQFMLDLHTEQHGYSENYVPYLVNQDTLYGTGQLPKFAGDLFHTRPLEEEADTSNYALIPSAGVPLTNLVRGEIIDEDDLPIKMTAHTPCFRSEAGSYGRDTRGLIRMHQFDKVEMVQIVRPEDSMAALEEMTGHAEKVLQLLGLPYRKIILCTGDMGFGACKTYDLEVWIPAQNTYREISSCSNVWDFQARRMQARCRSKSDKKTRLVHTLNGSGLAVGRTLVAVMENYQQADGRIEVPEVLRPYMNGLEYIG

SEQ ID NO: 10 - protein, 642 aa, synthetic construct
  REGION 1..642: note = artificially synthesized sequence
MPVITLPDGSQRHYDHAVSPMDVALDIGPGLAKACIAGRVNGELVDACDLIENDAQLSIITAKDEEGLEIIRHSCAHLLGHAIKQLWPHTKMAIGPVIDNGFYYDVDLDRTLTQEDVEALEKRMHELAEKNYDVIKKKVSWHEARETFANRGESYKVSILDENIAHDDKPGLYFHEEYVDMCRGPHVPNMRFCHHFKLMKTAGAYWRGDSNNKMLQRIYGTAWADKKALNAYLQRLEEAAKRDHRKIGKQLDLYHMQEEAPGMVFWHNDGWTIFRELEVFVRSKLKEYQYQEVKGPFMMDRVLWEKTGHWDNYKDAMFTTSSENREYCIKPGNCPGHVQIFNQGLKSYRDLPLRMAEFGSCHRNEPSGSLHGLMRVRGFTQDDAHIFCTEEQIRDEVNGCIRLVYDMYSTFGFEKIVVKLSTRPEKRIGSDEMWDRAEADLAVALEENNIPFEYQLGEGAFYGPKIEFTLYDCLDRAWQCGTVQLDFSLPSRLSASYVGEDNERKVPVMIHRAILGSMERFIGILTEEFAGFFPTWLAPVQVVIMNITDSQSEYVNELTQKLSNAGIRVKADLRNEKIGFKIREHTLRRVPYMLVCGDKEVESGKVAVRTRRGKDLGSMDVNEVIEKLQQEIRSRSLKQLEE

SEQ ID NO: 11 - protein, 642 aa, synthetic construct
  REGION 1..642: note = artificially synthesized sequence
MPVITLPDGSQRHYDHAVSPMDVALDIGPGLAKACIAGRVNGELVDACDLIENDAQLSIITAKDEEGLEIIRHSCAHLLGHAIKQLWPHTKMAIGPVIDNGFYYDVDLDRTLTQEDVEALEKRMHELAEKNYDVIKKKVSWHEARETFANRGESYKVSILDENIAHDDKPGLYFHEEYVDMCRGPHVPNMRFCHHFKLMKTAGAYWRGDSNNKMLQRIYGTAWADKKALNAYLQRLEEAAKRDHRKIGKQLDLYHMQEEAPGMVFWHNDGWTIFRELEVFVRSKLKEYQYQEVKGPFMMDRVLWEKTGHWDNYKDAMFTTSSENREYCIKPMNCPGHVQIFNQGLKSYRDLPLRMAEFGSCHRNEPSGSLHGLMRVRGFTQDDAHIFCTEEQIRDEVNGCIRLVYDMYSTFGFEKIVVKLSTRPEKRIGSDEMWDRAEADLAVALEENNIPFEYQLGEGAFYGPKIEFTLYDCLDRAWQCGTVQLDFSLPSRLSASYVGEDNERKVPVMIGRAILGSMERFIGILTEEFAGFFPTWLAPVQVVIMNITDSQSEYVNELTQKLSNAGIRVKADLRNEKIGFKIREHTLRRVPYMLVCGDKEVESGKVAVRTRRGKDLGSMDVNEVIEKLQQEIRSRSLKQLEE

SEQ ID NO: 12 - DNA, 981 bp, synthetic construct
  misc_feature 1..981: note = artificially synthesized sequence
  source 1..981: mol_type = other DNA
atgtcacatctcgcagaactggttgccagtgcgaaggcggccattagccaggcgtcagatgttgccgcgttagataatgtgcgcgtcgaatatttgggtaaaaaagggcacttaacccttcagatgacgaccctgcgtgagctgccgccagaagagcgtccggcagctggtgcggttatcaacgaagcgaaagagcaggttcagcaggcgctgaatgcgcgtaaagcggaactggaaagcgctgcactgaatgcgcgtctggcggcggaaacgattgatgtctctctgccaggtcgtcgcattgaaaacggcggtctgcatccggttacccgtaccatcgaccgtatcgaaagtttcttcggtgagcttggctttaccgtggcaaccgggccggaaatcgaagacgattatcataacttcgatgctctgaacattcctggtcaccacccggcgcgcgctgaccacgacactttctggtttgacactacccgcctgctgcgtaccgcgacctctggcgtacagatccgcaccatgaaagcccagcagccaccgattcgtatcatcgcgcctggccgtgtttatcgtaacgactacgaccagactcacacgccgatgttccatcagatggaaggtctgattgttgataccaacatcagctttaccaacctgaaaggcacgctgcacgacttcctgcgtaacttctttgaggaagatttgcagattcgcttccgtccttcctacttcccgtttaccgaaccttctgcagaagtggacgtcatgggtaaaaacggtaaatggctggaagtgctgggctgcgggatggtgcatccgaacgtgttgcgtaacgttggcatcgacccggaagtttactctggtttcgccttcgggatggggatggagcgtctgactatgttgcgttacggcgtcaccgacctgcgttcattcttcgaaaacgatctgcgtttcctcaaacagtttaaa

SEQ ID NO: 13 - DNA, 981 bp, synthetic construct
  misc_feature 1..981: note = artificially synthesized sequence
  source 1..981: mol_type = other DNA
atgtcacatctcgcagaactggttgccagtgcgaaggcggccattagccaggcgtcagatgttgccgcgttagataatgtgcgcgtcgaatatttgggtaaaaaagggcacttaacccttcagatgacgaccctgcgtgagctgccgccagaagagcgtccggcagctggtgcggttatcaacgaagcgaaagagcaggttcagcaggcgctgaatgcgcgtaaagcggaactggaaagcgctgcactgaatgcgcgtctggcggcggaaacgattgatgtctctctgccaggtcgtcgcattgaaaacggcggtctgcatccggttacccgtaccatcgaccgtatcgaaagtttcttcggtgagcttggctttaccgtggcaaccgggccggaaatcgaagacgattatcataacttcgatgctctgaacattcctggtcaccacccggcgcgcgctgaccacgacactttctggtttgacactacccgcctgctgcgtaccggcacctctggcgtacagatccgcaccatgaaagcccagcagccaccgattcgtatcatcgcgcctggccgtgtttatcgtaacgactacgaccagactcacacgccgatgttccatcagatggaaggtctgattgttgataccaacatcagctttaccaacctgaaaggcacgctgcacgacttcctgcgtaacttctttgaggaagatttgcagattcgcttccgtccttcctacttcccgtttaccgaaccttctgcagaagtggacgtcatgggtaaaaacggtaaatggctggaagtgctgggctgcgggatggtgcatccgaacgtgttgcgtaacgttggcatcgacccggaagtttactctggtttcgccttcgggatggggatggagcgtctgactatgttgcgttacggcgtcaccgacctgcgttcattcttcgaaaacgatctgcgtttcctcaaacagtttaaa

SEQ ID NO: 14 - DNA, 2853 bp, synthetic construct
  misc_feature 1..2853: note = artificially synthesized sequence
  source 1..2853: mol_type = other DNA
atggaaaagacatataacccacaagatatcgaacagccgctttacgagcactgggaaaagcagggctactttaagcctaatggcgatgaaagccaggaaagtttctgcatcatgatcccgccgccggctgtcaccggcagtttgcatatgggtcacgccttccagcaaaccatcatggataccatgatccgctatcagcgcatgcagggcaaaaacaccctgtggcaggtcggtactgaccacgccgggatcgctacccagatggtcgttgagcgcaagattgccgcagaagaaggtaaaacccgtcacgactacggccgcgaagctttcatcgacaaaatctgggaatggaaagcggaatctggcggcaccattacccgtcagatgcgccgtctcggcaactccgtcgactgggagcgtgaacgcttcaccatggacgaaggcctgtccaatgcggtgaaagaagttttcgttcgtctgtataaagaagacctgatttaccgtggcaaacgcctggtaaactgggatccgaaactgcgcaccgctatctctgacctggaagtcgaaaaccgcgaatcgaaaggttcgatgtggcacatccgctatccgctggctgacggtgcgaaaaccgcagacggtaaagattatctggtggtcgcgactacccgtccagaaaccctgctgggcgatactggcgtagccgttaacccggaagatccgcgttacaaagatctgattggcaaatatgtcattctgccgctggttaaccgtcgtattccgatcgttggcgacgaacacgccgacatggaaaaaggcaccggctgcgtgaaaatcactccggcgcacgactttaacgactatgaagtgggtaaacgtcacgccctgccgatgatcaacatcctgacctttgacggcgatatccgtgaaagcgcccaggtgttcgataccaaaggtaacgaatctgacgtttattccagcgaaatccctgcagagttccagaaactggagcgttttgctgcacgtaaagcagtcgttgccgcagttgacgcgcttggcctgctggaagaaattaaaccgcacgacctgaccgttccttacggcgaccgtggcggcgtagttatcgaaccaatgctgaccgaccagtggtacgtgcgtgccgatgtcctggcgaaaccggcggttgaagcggttgagaacggcgacattcagttcgtaccgaagcagtacgaaaacatgtacttctcctggatgcgcgatattcaggactggtgtatctctcgtcagttgtggtggggtcaccgtatcccggcatggtatgacgaagcgggtaacgtttatgttggccgcaacgaagacgaagtgcgtaaagaaaataacctcggtgctgatgttgtcctgcgtcaggacgaagacgttctcgatacctggttctcttctgcgctgtggaccttctctacccttggctggccggaaaataccgacgccctgcgtcagttccacccaaccagcgtgatggtatctggtttcgacatcattttcttctggattgcccgcatgatcatgatgaccatgcacttcatcaaagatgaaaatggcaaaccgcaggtgccgttccacaccgtttacatgaccggcctgattcgtgatgacgaaggccagaagatgtccaaatccaagggtaacgttatcgacccactggatatggttgacggtatttcgctgccagaactgctggaaaaacgtaccggcaatatgatgcagccgcagctggcggacaaaatccgtaagcgcaccgagaagcagttcccgaacggtattgagccgcacggtactgacgcgctgcgcttcaccctggcggcgctggcgtctaccggtcgtgacatcaactgggatatgaagcgtctggaaggttaccgtaacttctgtaacaagctgtggaacgccagccgctttgtgctgatgaacacagaaggtcaggattgcggcttcaacggcggcgaaatgacgctgtcgctggcggaccgctggattctggcggagttcaaccagaccatcaaagcgtaccgcgaagcgctggacagcttccgcttcgatatcgccgcaggcattctgtatgagttcacctggaaccagttctgtgactggtatctcgagctgaccaagccggtaatgaacggtggcaccgaagcagaactgcgcggtactcgccatacgctggtgactgtactggaaggtctgctgcgcctcgcgcatccgatcattccgttcatcaccgaaaccatctggcagcgtgtgaaagtactttgcggtatcactgccgacaccatcatgctgcagccgttcccgcagtacgatgcatctcaggttgatgaagccgcactggccgacaccgaatggctgaaacaggcgatcgttgcggtacgtaacatccgtgcagaaatgaacatcgcgccgggcaaaccgctggagctgctgctgcgtggttgcagcgcggatgcagaacgtcgcgtaaatgaaaaccgtggcttcctgcaaaccctggcgcgtctggaaagtatcaccgtgctgcctgccgatgacaaaggtccggtttccgttacgaagatcatcgacggtgcagagctgctgatcccgatggctggcctcatcaacaaagaagatgagctggcgcgtctggcgaaagaagtggcgaagattgaaggtgaaatcagccgtatcgagaacaaactggcgaacgaaggctttgtcgcccgcgcaccggaagcggtcatcgcgaaagagcgtgagaagctggaaggctatgcggaagcgaaagcgaaactgattgaacagcaggctgttatcgccgcgctg

SEQ ID NO: 15 - DNA, 2853 bp, synthetic construct
  misc_feature 1..2853: note = artificially synthesized sequence
  source 1..2853: mol_type = other DNA
atggaaaagacatataacccacaagatatcgaacagccgctttacgagcactgggaaaagcagggctactttaagcctaatggcgatgaaagccaggaaagtttctgcatcatgatcccgccgccgggtgtcaccggcagtttgcatatgggtcacgccttccagcaaaccatcatggataccatgatccgctatcagcgcatgcagggcaaaaacaccctgtggcaggtcggtactgaccacgccgggatcgctacccagatggtcgttgagcgcaagattgccgcagaagaaggtaaaacccgtcacgactacggccgcgaagctttcatcgacaaaatctgggaatggaaagcggaatctggcggcaccattacccgtcagatgcgccgtctcggcaactccgtcgactgggagcgtgaacgcttcaccatggacgaaggcctgtccaatgcggtgaaagaagttttcgttcgtctgtataaagaagacctgatttaccgtggcaaacgcctggtaaactgggatccgaaactgcgcaccgctatctctgacctggaagtcgaaaaccgcgaatcgaaaggttcgatgtggcacatccgctatccgctggctgacggtgcgaaaaccgcagacggtaaagattatctggtggtcgcgactacccgtccagaaaccctgctgggcgatactggcgtagccgttaacccggaagatccgcgttacaaagatctgattggcaaatatgtcattctgccgctggttaaccgtcgtattccgatcgttggcgacgaacacgccgacatggaaaaaggcaccggctgcgtgaaaatcactccggcgcacgactttaacgactatgaagtgggtaaacgtcacgccctgccgatgatcaacatcctgacctttgacggcgatatccgtgaaagcgcccaggtgttcgataccaaaggtaacgaatctgacgtttattccagcgaaatccctgcagagttccagaaactggagcgttttgctgcacgtaaagcagtcgttgccgcagttgacgcgcttggcctgctggaagaaattaaaccgcacgacctgaccgttccttacggcgaccgtggcggcgtagttatcgaaccaatgctgaccgaccagtggtacgtgcgtgccgatgtcctggcgaaaccggcggttgaagcggttgagaacggcgacattcagttcgtaccgaagcagtacgaaaacatgtacttctcctggatgcgcgatattcaggactggtgtatctctcgtcagttgtggtggggtcaccgtatcccggcatggtatgacgaagcgggtaacgtttatgttggccgcaacgaagacgaagtgcgtaaagaaaataacctcggtgctgatgttgtcctgcgtcaggacgaagacgttctcgatacctggttctcttctgcgctgtggaccttctctacccttggctggccggaaaataccgacgccctgcgtcagttccacccaaccagcgtgatggtatctggtttcgacatcattttcttctggattgcccgcatgatcatgatgaccatgcacttcatcaaagatgaaaatggcaaaccgcaggtgccgttccacaccgtttacatgaccggcctgattcgtgatgacgaaggccagaagatgtccaaatccaagggtaacgttatcgacccactggatatggttgacggtatttcgctgccagaactgctggaaaaacgtaccggcaatatgatgcagccgcagctggcggacaaaatccgtaagcgcaccgagaagcagttcccgaacggtattgagccgcacggtactgacgcgctgcgcttcaccctggcggcgctggcgtctaccggtcgtgacatcaactgggatatgaagcgtctggaaggttaccgtaacttctgtaacaagctgtggaacgccagccgctttgtgctgatgaacacagaaggtcaggattgcggcttcaacggcggcgaaatgacgctgtcgctggcggaccgctggattctggcggagttcaaccagaccatcaaagcgtaccgcgaagcgctggacagcttccgcttcgatatcgccgcaggcattctgtatgagttcacctggaaccagttctgtgactggtatctcgagctgaccaagccggtaatgaacggtggcaccgaagcagaactgcgcggtactcgccatacgctggtgactgtactggaaggtctgctgcgcctcgcgcatccgatcattccgttcatcaccgaaaccatctggcagcgtgtgaaagtactttgcggtatcactgccgacaccatcatgctgcagccgttcccgcagtacgatgcatctcaggttgatgaagccgcactggccgacaccgaatggctgaaacaggcgatcgttgcggtacgtaacatccgtgcagaaatgaacatcgcgccgggcaaaccgctggagctgctgctgcgtggttgcagcgcggatgcagaacgtcgcgtaaatgaaaaccgtggcttcctgcaaaccctggcgcgtctggaaagtatcaccgtgctgcctgccgatgacaaaggtccggtttccgttacgaagatcatcgacggtgcagagctgctgatcccgatggctggcctcatcaacaaagaagatgagctggcgcgtctggcgaaagaagtggcgaagattgaaggtgaaatcagccgtatcgagaacaaactggcgaacgaaggctttgtcgcccgcgcaccggaagcggtcatcgcgaaagagcgtgagaagctggaaggctatgcggaagcgaaagcgaaactgattgaacagcaggctgttatcgccgcgctg

SEQ ID NO: 16 - DNA, 2853 bp, synthetic construct
  misc_feature 1..2853: note = artificially synthesized sequence
  source 1..2853: mol_type = other DNA
atggaaaagacatataacccacaagatatcgaacagccgctttacgagcactgggaaaagcagggctactttaagcctaatggcgatgaaagccaggaaagtttctgcatcatgatcccgccgccgggtgtctcaggcagtttgcatatgggtcacgccttccagcaaaccatcatggataccatgatccgctatcagcgcatgcagggcaaaaacaccctgtggcaggtcggtactgaccacgccgggatcgctacccagatggtcgttgagcgcaagattgccgcagaagaaggtaaaacccgtcacgactacggccgcgaagctttcatcgacaaaatctgggaatggaaagcggaatctggcggcaccattacccgtcagatgcgccgtctcggcaactccgtcgactgggagcgtgaacgcttcaccatggacgaaggcctgtccaatgcggtgaaagaagttttcgttcgtctgtataaagaagacctgatttaccgtggcaaacgcctggtaaactgggatccgaaactgcgcaccgctatctctgacctggaagtcgaaaaccgcgaatcgaaaggttcgatgtggcacatccgctatccgctggctgacggtgcgaaaaccgcagacggtaaagattatctggtggtcgcgactacccgtccagaaaccctgctgggcgatactggcgtagccgttaacccggaagatccgcgttacaaagatctgattggcaaatatgtcattctgccgctggttaaccgtcgtattccgatcgttggcgacgaacacgccgacatggaaaaaggcaccggctgcgtgaaaatcactccggcgcacgactttaacgactatgaagtgggtaaacgtcacgccctgccgatgatcaacatcctgacctttgacggcgatatccgtgaaagcgcccaggtgttcgataccaaaggtaacgaatctgacgtttattccagcgaaatccctgcagagttccagaaactggagcgttttgctgcacgtaaagcagtcgttgccgcagttgacgcgcttggcctgctggaagaaattaaaccgcacgacctgaccgttccttacggcgaccgtggcggcgtagttatcgaaccaatgctgaccgaccagtggtacgtgcgtgccgatgtcctggcgaaaccggcggttgaagcggttgagaacggcgacattcagttcgtaccgaagcagtacgaaaacatgtacttctcctggatgcgcgatattcaggactggtgtatctctcgtcagttgtggtggggtcaccgtatcccggcatggtatgacgaagcgggtaacgtttatgttggccgcaacgaagacgaagtgcgtaaagaaaataacctcggtgctgatgttgtcctgcgtcaggacgaagacgttctcgatacctggttctcttctgcgctgtggaccttctctacccttggctggccggaaaataccgacgccctgcgtcagttccacccaaccagcgtgatggtatctggtttcgacatcattttcttctggattgcccgcatgatcatgatgaccatgcacttcatcaaagatgaaaatggcaaaccgcaggtgccgttccacaccgtttacatgaccggcctgattcgtgatgacgaaggccagaagatgtccaaatccaagggtaacgttatcgacccactggatatggttgacggtatttcgctgccagaactgctggaaaaacgtaccggcaatatgatgcagccgcagctggcggacaaaatccgtaagcgcaccgagaagcagttcccgaacggtattgagccgcacggtactgacgcgctgcgcttcaccctggcggcgctggcgtctaccggtcgtgacatcaactgggatatgaagcgtctggaaggttaccgtaacttctgtaacaagctgtggaacgccagccgctttgtgctgatgaacacagaaggtcaggattgcggcttcaacggcggcgaaatgacgctgtcgctggcggaccgctggattctggcggagttcaaccagaccatcaaagcgtaccgcgaagcgctggacagcttccgcttcgatatcgccgcaggcattctgtatgagttcacctggaaccagttctgtgactggtatctcgagctgaccaagccggtaatgaacggtggcaccgaagcagaactgcgcggtactcgccatacgctggtgactgtactggaaggtctgctgcgcctcgcgcatccgatcattccgttcatcaccgaaaccatctggcagcgtgtgaaagtactttgcggtatcactgccgacaccatcatgctgcagccgttcccgcagtacgatgcatctcaggttgatgaagccgcactggccgacaccgaatggctgaaacaggcgatcgttgcggtacgtaacatccgtgcagaaatgaacatcgcgccgggcaaaccgctggagctgctgctgcgtggttgcagcgcggatgcagaacgtcgcgtaaatgaaaaccgtggcttcctgcaaaccctggcgcgtctggaaagtatcaccgtgctgcctgccgatgacaaaggtccggtttccgttacgaagatcatcgacggtgcagagctgctgatcccgatggctggcctcatcaacaaagaagatgagctggcgcgtctggcgaaagaagtggcgaagattgaaggtgaaatcagccgtatcgagaacaaactggcgaacgaaggctttgtcgcccgcgcaccggaagcggtcatcgcgaaagagcgtgagaagctggaaggctatgcggaagcgaaagcgaaactgattgaacagcaggctgttatcgccgcgctg

SEQ ID NO: 17 - DNA, 1290 bp, synthetic construct
  misc_feature 1..1290: note = artificially synthesized sequence
  source 1..1290: mol_type = other DNA
atgctcgatcccaatctgctgcgtaatgagccagacgcagtcgctgaaaaactggcacgccggggctttaagctggatgtagataagctgggcgctcttgaagagcgtcgtaaagtattgcaggtcaaaacggaaaacctgcaagcggagcgtaactcccgatcgaaatccattggccaggcgaaagcgcgcggggaagatatcgagcctttacgtctggaagtgaacaaactgggcgaagagctggatgcagcaaaagccgagctggatgctttacaggctgaaattcgcgatatcgcgctgaccatccctaacctgcctgcagatgaagtgccggtaggtaaagacgaaaatgacaacgttgaagtcagccgctggggtaccccgcgtgagtttgactttgaagttcgtgaccacgtgacgctgggtgaaatgcactctggcctcgactttgcagctgcagttaagctgactggttcccgctttgtggtaatgaaagggcagattgctcgcatgcaccgcgcactgtcgcagtttatgctggatctgcataccgaacagcatggctacagtgagaactatgttccgtacctggttaaccaggacacgctgtacggtacgggtcaactgccgaaatttgctggcgatctgttccatactcgtccgctggaagaagaagcagacaccagtaactatgcgctgatcccaagtgcagaagttccgctgactaacctggtgcgcggtgaaatcatcgatgaagatgatctgccaattaagatgaccgcccacaccccatgcttccgttctgaagccggttcatatggtcgtgacacccgtggtctgatccgtatgcaccagttcgacaaagttgaaatggtgcagatcgtgcgcccagaagactcaatggcggcgctggaagagatgactggtcatgcagaaaaagtcctgcagttgctgggcctgccgtaccgtaaaatcatcctttgcactggcgacatgggctttggcgcttgcaaaacttacgacctggaagtatggatcccggcacagaacacctaccgtgagatctcttcctgctccaacgtttgggatttccaggcacgtcgtatgcaggcacgttgccgcagcaagtcggacaagaaaacccgtctggttcataccctgaacggttctggtctggctgttggtcgtacgctggttgcagtaatggaaaactatcagcaggctgatggtcgtattgaagtaccagaagttctgcgtccgtatatgaacggactggaatatattggc

SEQ ID NO: 18 - DNA, 1290 bp, synthetic construct
  misc_feature 1..1290: note = artificially synthesized sequence
  source 1..1290: mol_type = other DNA
atgctcgatcccaatctgctgcgtaatgagccagacgcagtcgctgaaaaactggcacgccggggctttaagctggatgtagataagctgggcgctcttgaagagcgtcgtaaagtattgcaggtcaaaacggaaaacctgcaagcggagcgtaactcccgatcgaaatccattggccaggcgaaagcgcgcggggaagatatcgagcctttacgtctggaagtgaacaaactgggcgaagagctggatgcagcaaaagccgagctggatgctttacaggctgaaattcgcgatatcgcgctgaccatccctaacctgcctgcagatgaagtgccggtaggtaaagacgaaaatgacaacgttgaagtcagccgctggggtaccccgcgtgagtttgactttgaagttcgtgaccacgtgacgctgggtgaaatgcactctggcctcgactttgcagctgcagttaagctgactggttcccgctttgtggtaatgaaagggcagattgctcgcatgcaccgcgcactgtcgcagtttatgctggatctgcataccgaacagcatggctacagtgagaactatgttccgtacctggttaaccaggacacgctgtacggtacgggtcaactgccgaaatttgctggcgatctgttccatactcgtccgctggaagaagaagcagacaccagtaactatgcgctgatcccaacggcagctgttccgctgactaacctggtgcgcggtgaaatcatcgatgaagatgatctgccaattaagatgaccgcccacaccccatgcttccgttctgaagccggttcatatggtcgtgacacccgtggtctgatccgtatgcaccagttcgacaaagttgaaatggtgcagatcgtgcgcccagaagactcaatggcggcgctggaagagatgactggtcatgcagaaaaagtcctgcagttgctgggcctgccgtaccgtaaaatcatcctttgcactggcgacatgggctttggcgcttgcaaaacttacgacctggaagtatggatcccggcacagaacacctaccgtgagatctcttcctgctccaacgtttgggatttccaggcacgtcgtatgcaggcacgttgccgcagcaagtcggacaagaaaacccgtctggttcataccctgaacggttctggtctggctgttggtcgtacgctggttgcagtaatggaaaactatcagcaggctgatggtcgtattgaagtaccagaagttctgcgtccgtatatgaacggactggaatatattggc

SEQ ID NO: 19 - DNA, 1290 bp, synthetic construct
  misc_feature 1..1290: note = artificially synthesized sequence
  source 1..1290: mol_type = other DNA
atgctcgatcccaatctgctgcgtaatgagccagacgcagtcgctgaaaaactggcacgccggggctttaagctggatgtagataagctgggcgctcttgaagagcgtcgtaaagtattgcaggtcaaaacggaaaacctgcaagcggagcgtaactcccgatcgaaatccattggccaggcgaaagcgcgcggggaagatatcgagcctttacgtctggaagtgaacaaactgggcgaagagctggatgcagcaaaagccgagctggatgctttacaggctgaaattcgcgatatcgcgctgaccatccctaacctgcctgcagatgaagtgccggtaggtaaagacgaaaatgacaacgttgaagtcagccgctggggtaccccgcgtgagtttgactttgaagttcgtgaccacgtgacgctgggtgaaatgcactctggcctcgactttgcagctgcagttaagctgactggttcccgctttgtggtaatgaaagggcagattgctcgcatgcaccgcgcactgtcgcagtttatgctggatctgcataccgaacagcatggctacagtgagaactatgttccgtacctggttaaccaggacacgctgtacggtacgggtcaactgccgaaatttgctggcgatctgttccatactcgtccgctggaagaagaagcagacaccagtaactatgcgctgatcccaacggcaggtgttccgctgactaacctggtgcgcggtgaaatcatcgatgaagatgatctgccaattaagatgaccgcccacaccccatgcttccgttctgaagccggttcatatggtcgtgacacccgtggtctgatccgtatgcaccagttcgacaaagttgaaatggtgcagatcgtgcgcccagaagactcaatggcggcgctggaagagatgactggtcatgcagaaaaagtcctgcagttgctgggcctgccgtaccgtaaaatcatcctttgcactggcgacatgggctttggcgcttgcaaaacttacgacctggaagtatggatcccggcacagaacacctaccgtgagatctcttcctgctccaacgtttgggatttccaggcacgtcgtatgcaggcacgttgccgcagcaagtcggacaagaaaacccgtctggttcataccctgaacggttctggtctggctgttggtcgtacgctggttgcagtaatggaaaactatcagcaggctgatggtcgtattgaagtaccagaagttctgcgtccgtatatgaacggactggaatatattggc

SEQ ID NO: 20 - DNA, 1290 bp, synthetic construct
  misc_feature 1..1290: note = artificially synthesized sequence
  source 1..1290: mol_type = other DNA
atgctcgatcccaatctgctgcgtaatgagccagacgcagtcgctgaaaaactggcacgccggggctttaagctggatgtagataagctgggcgctcttgaagagcgtcgtaaagtattgcaggtcaaaacggaaaacctgcaagcggagcgtaactcccgatcgaaatccattggccaggcgaaagcgcgcggggaagatatcgagcctttacgtctggaagtgaacaaactgggcgaagagctggatgcagcaaaagccgagctggatgctttacaggctgaaattcgcgatatcgcgctgaccatccctaacctgcctgcagatgaagtgccggtaggtaaagacgaaaatgacaacgttgaagtcagccgctggggtaccccgcgtgagtttgactttgaagttcgtgaccacgtgacgctgggtgaaatgcactctggcctcgactttgcagctgcagttaagctgactggttcccgctttgtggtaatgaaagggcagattgctcgcatgcaccgcgcactgtcgcagtttatgctggatctgcataccgaacagcatggctacagtgagaactatgttccgtacctggttaaccaggacacgctgtacggtacgggtcaactgccgaaatttgctggcgatctgttccatactcgtccgctggaagaagaagcagacaccagtaactatgcgctgatcccaagtgcaggtgttccgctgactaacctggtgcgcggtgaaatcatcgatgaagatgatctgccaattaagatgaccgcccacaccccatgcttccgttctgaagccggttcatatggtcgtgacacccgtggtctgatccgtatgcaccagttcgacaaagttgaaatggtgcagatcgtgcgcccagaagactcaatggcggcgctggaagagatgactggtcatgcagaaaaagtcctgcagttgctgggcctgccgtaccgtaaaatcatcctttgcactggcgacatgggctttggcgcttgcaaaacttacgacctggaagtatggatcccggcacagaacacctaccgtgagatctcttcctgctccaacgtttgggatttccaggcacgtcgtatgcaggcacgttgccgcagcaagtcggacaagaaaacccgtctggttcataccctgaacggttctggtctggctgttggtcgtacgctggttgcagtaatggaaaactatcagcaggctgatggtcgtattgaagtaccagaagttctgcgtccgtatatgaacggactggaatatattggc

SEQ ID NO: 21 - DNA, 1926 bp, synthetic construct
  misc_feature 1..1926: note = artificially synthesized sequence
  source 1..1926: mol_type = other DNA
atgccggttattaccctgccggacggttcgcaacgtcattacgatcacgctgttagcccgatggatgtcgcactggatattggcccgggtctggcaaaagcttgcattgcaggccgcgttaatggtgaactggtcgacgcttgtgatctgatcgaaaacgacgcgcagctgagcattatcaccgccaaagatgaagaaggcctggaaattatccgtcattcttgcgcgcacctgctgggtcatgccattaaacagctgtggccgcataccaaaatggcgattggcccggtcatcgacaatggtttttattacgatgtggacctggatcgcaccctgacgcaggaagatgtggaagcgctggaaaaacgtatgcacgaactggccgagaaaaactacgacgtgattaagaaaaaagttagttggcatgaagcacgtgaaacctttgctaatcgcggcgaatcatacaaagtttcgattctggatgaaaacatcgcccacgatgacaaaccgggcctgtatttccatgaagaatacgtggatatgtgccgtggtccgcacgttccgaatatgcgcttttgtcatcacttcaaactgatgaaaaccgcaggtgcatattggcgtggtgacagcaacaataaaatgctgcagcgtatttatggtacggcatgggctgataaaaaagcgctgaatgcctacctgcaacgcctggaagaagcggccaaacgtgaccaccgcaaaattggcaaacagctggatctgtatcatatgcaagaagaagcgccgggcatggtgttttggcataacgatggttggaccatctttcgtgaactggaagttttcgtccgcagcaaactgaaagaatatcagtaccaagaagttaaaggcccgtttatgatggatcgcgtcctgtgggaaaaaaccggtcactgggacaactataaagatgccatgttcaccacgagctctgaaaaccgtgaatactgcattaaaccgggcaattgtccgggtcatgtgcagatctttaaccaaggcctgaaaagttatcgtgatctgccgctgcgcatggcagaattcggttcctgccaccgcaatgaaccgagtggctccctgcatggtctgatgcgtgttcgcggttttacccaggatgacgcccatattttctgcacggaagaacaaatccgtgacgaagttaacggctgtattcgcctggtctatgatatgtactctacctttggtttcgaaaaaattgtggttaaactgagcacgcgtccggaaaaacgcatcggctctgacgaaatgtgggatcgtgcggaagccgatctggcagtggctctggaagaaaacaatattccgtttgaatatcagctgggcgagggtgcgttttacggcccgaaaatcgaattcaccctgtatgactgcctggatcgcgcctggcagtgtggtacggtccaactggacttcagtctgccgtcccgtctgtcagcatcgtacgtgggcgaagataatgaacgtaaagtgccggttatgattcaccgcgctatcctgggcagtatggaacgttttattggtatcctgaccgaagaattcgcaggctttttcccgacgtggctggctccggttcaggtcgtgattatgaatatcaccgatagccagtctgaatatgtgaacgaactgacgcaaaaactgtccaatgcaggcattcgtgttaaagctgatctgcgcaacgaaaaaatcggtttcaaaatccgcgaacataccctgcgtcgcgtcccgtacatgctggtgtgtggcgataaagaagttgaaagcggtaaagtcgcggtgcgtacgcgtcgcggcaaagacctgggttcgatggatgtcaacgaagtcattgaaaaactgcaacaagaaatccgtagccgctcgctgaaacaactggaagaa

SEQ ID NO: 22 - DNA, 1926 bp, synthetic construct
  misc_feature 1..1926: note = artificially synthesized sequence
  source 1..1926: mol_type = other DNA
atgccggtcattaccctgccggatggctctcagcgtcactacgatcatgcggtctcgccgatggatgttgctctggatattggcccgggtctggcaaaagcttgcattgcaggccgcgttaatggtgaactggtcgacgcttgtgatctgatcgaaaacgacgcgcagctgagcattatcaccgccaaagatgaagaaggcctggaaattatccgtcattcttgcgcgcacctgctgggtcatgccattaagcagctgtggccgcataccaaaatggcgattggcccggtcatcgacaatggtttttattacgatgtggacctggatcgcaccctgacgcaggaagatgtggaagcgctggaaaagcgtatgcacgaactggccgaaaagaactatgacgtgatcaaaaagaaagttagttggcatgaagcacgtgaaacctttgctaatcgcggcgaatcatacaaggtttcgattctggatgaaaacatcgcccacgatgacaaaccgggcctgtatttccatgaagaatacgtggatatgtgccgtggtccgcacgttccgaatatgcgcttttgtcatcacttcaagctgatgaaaaccgcaggtgcatattggcgtggtgacagcaacaataaaatgctgcagcgtatttatggtacggcatgggctgataagaaagcgctgaatgcctacctgcaacgcctggaagaagcggccaaacgtgaccaccgcaagattggcaaacagctggatctgtatcatatgcaagaagaagcgccgggcatggtgttttggcataacgatggttggaccatctttcgtgaactggaagttttcgtccgcagcaagctgaaagaatatcagtaccaagaagttaaaggcccgtttatgatggatcgcgtcctgtgggaaaagaccggtcactgggacaactataaagatgccatgttcaccacgagctctgaaaaccgtgaatactgcattaagccgatgaattgtccgggccatgtgcagatctttaaccaaggtctgaaaagttatcgtgatctgccgctgcgcatggcagaattcggttcctgccaccgcaatgaaccgagtggctccctgcatggtctgatgcgtgttcgcggctttacccaggatgacgcccacattttctgcacggaagaacaaatccgtgacgaagttaacggctgtattcgcctggtctatgatatgtactctacctttggtttcgaaaagattgtggttaaactgagcacgcgtccggaaaaacgcatcggttctgacgaaatgtgggatcgtgcggaagccgatctggcagtggctctggaagaaaacaatattccgtttgaatatcagctgggcgagggtgcgttttacggcccgaaaatcgaattcaccctgtatgactgcctggatcgcgcctggcagtgtggtacggtccaactggacttcagtctgccgtcccgtctgtcagcatcgtacgtgggcgaagataatgaacgtaaagtgccggttatgattggccgcgctatcctgggtagtatggaacgttttattggcatcctgaccgaagaattcgcaggctttttcccgacgtggctggctccggttcaggtcgtgattatgaatatcaccgatagccagtctgaatatgtgaacgaactgacgcaaaagctgtccaatgcaggcattcgtgttaaagctgatctgcgcaacgaaaagattggttttaaaatccgcgaacataccctgcgtcgcgtcccgtacatgctggtgtgtggcgataaggaagttgaaagcggtaaagtcgcggtgcgtacgcgtcgcggcaaagacctgggttcgatggatgtcaacgaagtcattgaaaaactgcaacaagaaatccgctcacgctcgctgaaacaactggaagaa

SEQ ID NO: 23 - DNA, 2853 bp, synthetic construct
  source 1..2853: mol_type = other DNA
atggaaaagacatataacccacaagatatcgaacagccgctttacgagcactgggaaaagcagggctactttaagcctaatggcgatgaaagccaggaaagtttctgcatcatgatcccgccgccgaacgtcaccggcagtttgcatatgggtcacgccttccagcaaaccatcatggataccatgatccgctatcagcgcatgcagggcaaaaacaccctgtggcaggtcggtactgaccacgccgggatcgctacccagatggtcgttgagcgcaagattgccgcagaagaaggtaaaacccgtcacgactacggccgcgaagctttcatcgacaaaatctgggaatggaaagcggaatctggcggcaccattacccgtcagatgcgccgtctcggcaactccgtcgactgggagcgtgaacgcttcaccatggacgaaggcctgtccaatgcggtgaaagaagttttcgttcgtctgtataaagaagacctgatttaccgtggcaaacgcctggtaaactgggatccgaaactgcgcaccgctatctctgacctggaagtcgaaaaccgcgaatcgaaaggttcgatgtggcacatccgctatccgctggctgacggtgcgaaaaccgcagacggtaaagattatctggtggtcgcgactacccgtccagaaaccctgctgggcgatactggcgtagccgttaacccggaagatccgcgttacaaagatctgattggcaaatatgtcattctgccgctggttaaccgtcgtattccgatcgttggcgacgaacacgccgacatggaaaaaggcaccggctgcgtgaaaatcactccggcgcacgactttaacgactatgaagtgggtaaacgtcacgccctgccgatgatcaacatcctgacctttgacggcgatatccgtgaaagcgcccaggtgttcgataccaaaggtaacgaatctgacgtttattccagcgaaatccctgcagagttccagaaactggagcgttttgctgcacgtaaagcagtcgttgccgcagttgacgcgcttggcctgctggaagaaattaaaccgcacgacctgaccgttccttacggcgaccgtggcggcgtagttatcgaaccaatgctgaccgaccagtggtacgtgcgtgccgatgtcctggcgaaaccggcggttgaagcggttgagaacggcgacattcagttcgtaccgaagcagtacgaaaacatgtacttctcctggatgcgcgatattcaggactggtgtatctctcgtcagttgtggtggggtcaccgtatcccggcatggtatgacgaagcgggtaacgtttatgttggccgcaacgaagacgaagtgcgtaaagaaaataacctcggtgctgatgttgtcctgcgtcaggacgaagacgttctcgatacctggttctcttctgcgctgtggaccttctctacccttggctggccggaaaataccgacgccctgcgtcagttccacccaaccagcgtgatggtatctggtttcgacatcattttcttctggattgcccgcatgatcatgatgaccatgcacttcatcaaagatgaaaatggcaaaccgcaggtgccgttccacaccgtttacatgaccggcctgattcgtgatgacgaaggccagaagatgtccaaatccaagggtaacgttatcgacccactggatatggttgacggtatttcgctgccagaactgctggaaaaacgtaccggcaatatgatgcagccgcagctggcggacaaaatccgtaagcgcaccgagaagcagttcccgaacggtattgagccgcacggtactgacgcgctgcgcttcaccctggcggcgctggcgtctaccggtcgtgacatcaactgggatatgaagcgtctggaaggttaccgtaacttctgtaacaagctgtggaacgccagccgctttgtgctgatgaacacagaaggtcaggattgcggcttcaacggcggcgaaatgacgctgtcgctggcggaccgctggattctggcggagttcaaccagaccatcaaagcgtaccgcgaagcgctggacagcttccgcttcgatatcgccgcaggcattctgtatgagttcacctggaaccagttctgtgactggtatctcgagctgaccaagccggtaatgaacggtggcaccgaagcagaactgcgcggtactcgccatacgctggtgactgtactggaaggtctgctgcgcctcgcgcatccgatcattccgttcatcaccgaaaccatctggcagcgtgtgaaagtactttgcggtatcactgccgacaccatcatgctgcagccgttcccgcagtacgatgcatctcaggttgatgaagccgcactggccgacaccgaatggctgaaacaggcgatcgttgcggtacgtaacatccgtgcagaaatgaacatcgcgccgggcaaaccgctggagctgctgctgcgtggttgcagcgcggatgcagaacgtcgcgtaaatgaaaaccgtggcttcctgcaaaccctggcgcgtctggaaagtatcaccgtgctgcctgccgatgacaaaggtccggtttccgttacgaagatcatcgacggtgcagagctgctgatcccgatggctggcctcatcaacaaagaagatgagctggcgcgtctggcgaaagaagtggcgaagattgaaggtgaaatcagccgtatcgagaacaaactggcgaacgaaggctttgtcgcccgcgcaccggaagcggtcatcgcgaaagagcgtgagaagctggaaggctatgcggaagcgaaagcgaaactgattgaacagcaggctgttatcgccgcgctg

SEQ ID NO: 24 - protein, 951 aa, synthetic construct
MEKTYNPQDIEQPLYEHWEKQGYFKPNGDESQESFCIMIPPPNVTGSLHMGHAFQQTIMDTMIRYQRMQGKNTLWQVGTDHAGIATQMVVERKIAAEEGKTRHDYGREAFIDKIWEWKAESGGTITRQMRRLGNSVDWERERFTMDEGLSNAVKEVFVRLYKEDLIYRGKRLVNWDPKLRTAISDLEVENRESKGSMWHIRYPLADGAKTADGKDYLVVATTRPETLLGDTGVAVNPEDPRYKDLIGKYVILPLVNRRIPIVGDEHADMEKGTGCVKITPAHDFNDYEVGKRHALPMINILTFDGDIRESAQVFDTKGNESDVYSSEIPAEFQKLERFAARKAVVAAVDALGLLEEIKPHDLTVPYGDRGGVVIEPMLTDQWYVRADVLAKPAVEAVENGDIQFVPKQYENMYFSWMRDIQDWCISRQLWWGHRIPAWYDEAGNVYVGRNEDEVRKENNLGADVVLRQDEDVLDTWFSSALWTFSTLGWPENTDALRQFHPTSVMVSGFDIIFFWIARMIMMTMHFIKDENGKPQVPFHTVYMTGLIRDDEGQKMSKSKGNVIDPLDMVDGISLPELLEKRTGNMMQPQLADKIRKRTEKQFPNGIEPHGTDALRFTLAALASTGRDINWDMKRLEGYRNFCNKLWNASRFVLMNTEGQDCGFNGGEMTLSLADRWILAEFNQTIKAYREALDSFRFDIAAGILYEFTWNQFCDWYLELTKPVMNGGTEAELRGTRHTLVTVLEGLLRLAHPIIPFITETIWQRVKVLCGITADTIMLQPFPQYDASQVDEAALADTEWLKQAIVAVRNIRAEMNIAPGKPLELLLRGCSADAERRVNENRGFLQTLARLESITVLPADDKGPVSVTKIIDGAELLIPMAGLINKEDELARLAKEVAKIEGEISRIENKLANEGFVARAPEAVIAKEREKLEGYAEAKAKLIEQQAVIAAL

SEQ ID NO: 25 - DNA, 1290 bp, synthetic construct
  source 1..1290: mol_type = other DNA
atgctcgatcccaatctgctgcgtaatgagccagacgcagtcgctgaaaaactggcacgccggggctttaagctggatgtagataagctgggcgctcttgaagagcgtcgtaaagtattgcaggtcaaaacggaaaacctgcaagcggagcgtaactcccgatcgaaatccattggccaggcgaaagcgcgcggggaagatatcgagcctttacgtctggaagtgaacaaactgggcgaagagctggatgcagcaaaagccgagctggatgctttacaggctgaaattcgcgatatcgcgctgaccatccctaacctgcctgcagatgaagtgccggtaggtaaagacgaaaatgacaacgttgaagtcagccgctggggtaccccgcgtgagtttgactttgaagttcgtgaccacgtgacgctgggtgaaatgcactctggcctcgactttgcagctgcagttaagctgactggttcccgctttgtggtaatgaaagggcagattgctcgcatgcaccgcgcactgtcgcagtttatgctggatctgcataccgaacagcatggctacagtgagaactatgttccgtacctggttaaccaggacacgctgtacggtacgggtcaactgccgaaatttgctggcgatctgttccatactcgtccgctggaagaagaagcagacaccagtaactatgcgctgatcccaacggcagaagttccgctgactaacctggtgcgcggtgaaatcatcgatgaagatgatctgccaattaagatgaccgcccacaccccatgcttccgttctgaagccggttcatatggtcgtgacacccgtggtctgatccgtatgcaccagttcgacaaagttgaaatggtgcagatcgtgcgcccagaagactcaatggcggcgctggaagagatgactggtcatgcagaaaaagtcctgcagttgctgggcctgccgtaccgtaaaatcatcctttgcactggcgacatgggctttggcgcttgcaaaacttacgacctggaagtatggatcccggcacagaacacctaccgtgagatctcttcctgctccaacgtttgggatttccaggcacgtcgtatgcaggcacgttgccgcagcaagtcggacaagaaaacccgtctggttcataccctgaacggttctggtctggctgttggtcgtacgctggttgcagtaatggaaaactatcagcaggctgatggtcgtattgaagtaccagaagttctgcgtccgtatatgaacggactggaatatattggc

SEQ ID NO: 26 - protein, 430 aa, synthetic construct
MLDPNLLRNEPDAVAEKLARRGFKLDVDKLGALEERRKVLQVKTENLQAERNSRSKSIGQAKARGEDIEPLRLEVNKLGEELDAAKAELDALQAEIRDIALTIPNLPADEVPVGKDENDNVEVSRWGTPREFDFEVRDHVTLGEMHSGLDFAAAVKLTGSRFVVMKGQIARMHRALSQFMLDLHTEQHGYSENYVPYLVNQDTLYGTGQLPKFAGDLFHTRPLEEEADTSNYALIPTAEVPLTNLVRGEIIDEDDLPIKMTAHTPCFRSEAGSYGRDTRGLIRMHQFDKVEMVQIVRPEDSMAALEEMTGHAEKVLQLLGLPYRKIILCTGDMGFGACKTYDLEVWIPAQNTYREISSCSNVWDFQARRMQARCRSKSDKKTRLVHTLNGSGLAVGRTLVAVMENYQQADGRIEVPEVLRPYMNGLEYIG

SEQ ID NO: 27 - DNA, 981 bp, synthetic construct
  source 1..981: mol_type = other DNA
atgtcacatctcgcagaactggttgccagtgcgaaggcggccattagccaggcgtcagatgttgccgcgttagataatgtgcgcgtcgaatatttgggtaaaaaagggcacttaacccttcagatgacgaccctgcgtgagctgccgccagaagagcgtccggcagctggtgcggttatcaacgaagcgaaagagcaggttcagcaggcgctgaatgcgcgtaaagcggaactggaaagcgctgcactgaatgcgcgtctggcggcggaaacgattgatgtctctctgccaggtcgtcgcattgaaaacggcggtctgcatccggttacccgtaccatcgaccgtatcgaaagtttcttcggtgagcttggctttaccgtggcaaccgggccggaaatcgaagacgattatcataacttcgatgctctgaacattcctggtcaccacccggcgcgcgctgaccacgacactttctggtttgacactacccgcctgctgcgtacccagacctctggcgtacagatccgcaccatgaaagcccagcagccaccgattcgtatcatcgcgcctggccgtgtttatcgtaacgactacgaccagactcacacgccgatgttccatcagatggaaggtctgattgttgataccaacatcagctttaccaacctgaaaggcacgctgcacgacttcctgcgtaacttctttgaggaagatttgcagattcgcttccgtccttcctacttcccgtttaccgaaccttctgcagaagtggacgtcatgggtaaaaacggtaaatggctggaagtgctgggctgcgggatggtgcatccgaacgtgttgcgtaacgttggcatcgacccggaagtttactctggtttcgccttcgggatggggatggagcgtctgactatgttgcgttacggcgtcaccgacctgcgttcattcttcgaaaacgatctgcgtttcctcaaacagtttaaa

SEQ ID NO: 28 - protein, 327 aa, synthetic construct
MSHLAELVASAKAAISQASDVAALDNVRVEYLGKKGHLTLQMTTLRELPPEERPAAGAVINEAKEQVQQALNARKAELESAALNARLAAETIDVSLPGRRIENGGLHPVTRTIDRIESFFGELGFTVATGPEIEDDYHNFDALNIPGHHPARADHDTFWFDTTRLLRTQTSGVQIRTMKAQQPPIRIIAPGRVYRNDYDQTHTPMFHQMEGLIVDTNISFTNLKGTLHDFLRNFFEEDLQIRFRPSYFPFTEPSAEVDVMGKNGKWLEVLGCGMVHPNVLRNVGIDPEVYSGFAFGMGMERLTMLRYGVTDLRSFFENDLRFLKQFK

SEQ ID NO: 29 - protein, 642 aa, synthetic construct
MPVITLPDGSQRHYDHAVSPMDVALDIGPGLAKACIAGRVNGELVDACDLIENDAQLSIITAKDEEGLEIIRHSCAHLLGHAIKQLWPHTKMAIGPVIDNGFYYDVDLDRTLTQEDVEALEKRMHELAEKNYDVIKKKVSWHEARETFANRGESYKVSILDENIAHDDKPGLYFHEEYVDMCRGPHVPNMRFCHHFKLMKTAGAYWRGDSNNKMLQRIYGTAWADKKALNAYLQRLEEAAKRDHRKIGKQLDLYHMQEEAPGMVFWHNDGWTIFRELEVFVRSKLKEYQYQEVKGPFMMDRVLWEKTGHWDNYKDAMFTTSSENREYCIKPMNCPGHVQIFNQGLKSYRDLPLRMAEFGSCHRNEPSGSLHGLMRVRGFTQDDAHIFCTEEQIRDEVNGCIRLVYDMYSTFGFEKIVVKLSTRPEKRIGSDEMWDRAEADLAVALEENNIPFEYQLGEGAFYGPKIEFTLYDCLDRAWQCGTVQLDFSLPSRLSASYVGEDNERKVPVMIHRAILGSMERFIGILTEEFAGFFPTWLAPVQVVIMNITDSQSEYVNELTQKLSNAGIRVKADLRNEKIGFKIREHTLRRVPYMLVCGDKEVESGKVAVRTRRGKDLGSMDVNEVIEKLQQEIRSRSLKQLEE

SEQ ID NO: 30 - DNA, 41 bp, synthetic construct
  misc_feature 1..41: note = artificially synthesized sequence
  source 1..41: mol_type = other DNA
gcctgctgcgtacccagaccgcgggcgtacagatccgcacc

SEQ ID NO: 31 - DNA, 41 bp, synthetic construct
  misc_feature 1..41: note = artificially synthesized sequence
  source 1..41: mol_type = other DNA
gcctgctgcgtacccagaccggcggcgtacagatccgcacc

SEQ ID NO: 32 - DNA, 42 bp, synthetic construct
  misc_feature 1..42: note = artificially synthesized sequence
  source 1..42: mol_type = other DNA
ctacccgcctgctgcgtaccgcgacctctggcgtacagatcc

SEQ ID NO: 33 - DNA, 42 bp, synthetic construct
  misc_feature 1..42: note = artificially synthesized sequence
  source 1..42: mol_type = other DNA
ctacccgcctgctgcgtaccggcacctctggcgtacagatcc

SEQ ID NO: 34 - DNA, 41 bp, synthetic construct
  misc_feature 1..41: note = artificially synthesized sequence
  source 1..41: mol_type = other DNA
ggtgcggatctgtacgcccgcggtctgggtacgcagcaggc

SEQ ID NO: 35 - DNA, 41 bp, synthetic construct
  misc_feature 1..41: note = artificially synthesized sequence
  source 1..41: mol_type = other DNA
ggtgcggatctgtacgccgccggtctgggtacgcagcaggc

SEQ ID NO: 36 - DNA, 42 bp, synthetic construct
  misc_feature 1..42: note = artificially synthesized sequence
  source 1..42: mol_type = other DNA
ggatctgtacgccagaggtcgcggtacgcagcaggcgggtag

SEQ ID NO: 37 - DNA, 42 bp, synthetic construct
  misc_feature 1..42: note = artificially synthesized sequence
  source 1..42: mol_type = other DNA
ggatctgtacgccagaggtgccggtacgcagcaggcgggtag

SEQ ID NO: 38 - DNA, 97 bp, synthetic construct
  misc_feature 1..97: note = artificially synthesized sequence
  source 1..97: mol_type = other DNA
ggcgtaatacgactcactatagcccggatagctcagtcggtagagcaggggattgaaaatccccgtgtccttggttcgattccgagtccgggcacca

SEQ ID NO: 39 - RNA, 76 nt, synthetic construct
  misc_feature 1..76: note = artificially synthesized sequence
  source 1..76: mol_type = other RNA
gcccggatagctcagtcggtagagcaggggattgaaaatccccgtgtccttggttcgattccgagtccgggcacca

SEQ ID NO: 40 - DNA, 92 bp, synthetic construct
  misc_feature 1..92: note = artificially synthesized sequence
  source 1..92: mol_type = other DNA
ggcgtaatacgactcactatagggttaactttaacaaggagaaaaacatgcgtttccgtgactacaaggacgacgacgacaagtaagcttcg

SEQ ID NO: 41 - RNA, 71 nt, synthetic construct
  misc_feature 1..71: note = artificially synthesized sequence
  source 1..71: mol_type = other RNA
gggttaactttaacaaggagaaaaacatgcgtttccgtgactacaaggacgacgacgacaagtaagcttcg

SEQ ID NO: 42 - protein, 12 aa, synthetic construct
  REGION 1..12: note = artificially synthesized sequence
  SITE 3: note = MISC_FEATURE - Xaa is phenylalanine or methyl phenylalanine
MRXRDYKDDDDK

SEQ ID NO: 43 - DNA, 49 bp, synthetic construct
  misc_feature 1..49: note = artificially synthesized sequence
  source 1..49: mol_type = other DNA
gtttctgcatcatgatcccggctccgaacgtcaccggcagtttgcatat

SEQ ID NO: 44 - DNA, 49 bp, synthetic construct
  misc_feature 1..49: note = artificially synthesized sequence
  source 1..49: mol_type = other DNA
gtttctgcatcatgatcccgggtccgaacgtcaccggcagtttgcatat

SEQ ID NO: 45 - DNA, 49 bp, synthetic construct
  misc_feature 1..49: note = artificially synthesized sequence
  source 1..49: mol_type = other DNA
gtttctgcatcatgatcccgccgccggctgtcaccggcagtttgcatat

SEQ ID NO: 46 - DNA, 49 bp, synthetic construct
  misc_feature 1..49: note = artificially synthesized sequence
  source 1..49: mol_type = other DNA
gtttctgcatcatgatcccggctccggctgtcaccggcagtttgcatat

SEQ ID NO: 47 - DNA, 49 bp, synthetic construct
  misc_feature 1..49: note = artificially synthesized sequence
  source 1..49: mol_type = other DNA
gtttctgcatcatgatcccgggtccggctgtcaccggcagtttgcatat

SEQ ID NO: 48 - DNA, 49 bp, synthetic construct
  misc_feature 1..49: note = artificially synthesized sequence
  source 1..49: mol_type = other DNA
gtttctgcatcatgatcccgccgccgagtgtcaccggcagtttgcatat

SEQ ID NO: 49 - DNA, 49 bp, synthetic construct
  misc_feature 1..49: note = artificially synthesized sequence
  source 1..49: mol_type = other DNA
gtttctgcatcatgatcccggctccgagtgtcaccggcagtttgcatat

SEQ ID NO: 50 - DNA, 49 bp, synthetic construct
  misc_feature 1..49: note = artificially synthesized sequence
  source 1..49: mol_type = other DNA
gtttctgcatcatgatcccgggtccgagtgtcaccggcagtttgcatat

SEQ ID NO: 51 - DNA, 49 bp, synthetic construct
  misc_feature 1..49: note = artificially synthesized sequence
  source 1..49: mol_type = other DNA
gtttctgcatcatgatcccgccgccggtagtcaccggcagtttgcatat

SEQ ID NO: 52 - DNA, 49 bp, synthetic construct
  misc_feature 1..49: note = artificially synthesized sequence
  source 1..49: mol_type = other DNA
gtttctgcatcatgatcccggctccggtagtcaccggcagtttgcatat

SEQ ID NO: 53 - DNA, 49 bp, synthetic construct
  misc_feature 1..49: note = artificially synthesized sequence
  source 1..49: mol_type = other DNA
gtttctgcatcatgatcccgggtccggtagtcaccggcagtttgcatat

SEQ ID NO: 54 - DNA, 49 bp, synthetic construct
  misc_feature 1..49: note = artificially synthesized sequence
  source 1..49: mol_type = other DNA
gtttctgcatcatgatcccgccgccgggtgtcaccggcagtttgcatat

SEQ ID NO: 55 - DNA, 49 bp, synthetic construct
  misc_feature 1..49: note = artificially synthesized sequence
  source 1..49: mol_type = other DNA
gtttctgcatcatgatcccggctccgggtgtcaccggcagtttgcatat

SEQ ID NO: 56 - DNA, 49 bp, synthetic construct
  misc_feature 1..49: note = artificially synthesized sequence
  source 1..49: mol_type = other DNA
gtttctgcatcatgatcccgggtccgggtgtcaccggcagtttgcatat

SEQ ID NO: 57 - DNA, 49 bp, synthetic construct
  misc_feature 1..49: note = artificially synthesized sequence
  source 1..49: mol_type = other DNA
gtttctgcatcatgatcccgccgccggatgtcaccggcagtttgcatat

SEQ ID NO: 58 - DNA, 49 bp, synthetic construct
  misc_feature 1..49: note = artificially synthesized sequence
  source 1..49: mol_type = other DNA
gtttctgcatcatgatcccggctccggatgtcaccggcagtttgcatat

SEQ ID NO: 59 - DNA, 49 bp, synthetic construct
  misc_feature 1..49: note = artificially synthesized sequence
  source 1..49: mol_type = other DNA
gtttctgcatcatgatcccgggtccggatgtcaccggcagtttgcatat

SEQ ID NO: 60 - DNA, 33 bp, synthetic construct
  misc_feature 1..33: note = artificially synthesized sequence
  source 1..33: mol_type = other DNA
tggcaggtcggtactgctcacgccgggatcgct

SEQ ID NO: 61 - DNA, 33 bp, synthetic construct
  misc_feature 1..33: note = artificially synthesized sequence
  source 1..33: mol_type = other DNA
tggcaggtcggtactagtcacgccgggatcgct

SEQ ID NO: 62 - DNA, 33 bp, synthetic construct
  misc_feature 1..33: note = artificially synthesized sequence
  source 1..33: mol_type = other DNA
tggcaggtcggtactgtacacgccgggatcgct

SEQ ID NO: 63 - DNA, 33 bp, synthetic construct
  misc_feature 1..33: note = artificially synthesized sequence
  source 1..33: mol_type = other DNA
tggcaggtcggtactggtcacgccgggatcgct

SEQ ID NO: 64 - DNA, 43 bp, synthetic construct
  misc_feature 1..43: note = artificially synthesized sequence
  source 1..43: mol_type = other DNA
gtactgaccacgccgggatctatacccagatggtcgttgagcg

SEQ ID NO: 65 - DNA, 43 bp, synthetic construct
  misc_feature 1..43: note = artificially synthesized sequence
  source 1..43: mol_type = other DNA
gtactgaccacgccgggatctggacccagatggtcgttgagcg

SEQ ID NO: 66 - DNA, 43 bp, synthetic construct
  misc_feature 1..43: note = artificially synthesized sequence
  source 1..43: mol_type = other DNA
gtactgaccacgccgggatctcaacccagatggtcgttgagcg

SEQ ID NO: 67 - DNA, 43 bp, synthetic construct
  misc_feature 1..43: note = artificially synthesized sequence
  source 1..43: mol_type = other DNA
gtactgaccacgccgggatcatgacccagatggtcgttgagcg

SEQ ID NO: 68 - DNA, 43 bp, synthetic construct
  misc_feature 1..43: note = artificially synthesized sequence
  source 1..43: mol_type = other DNA
gtactgaccacgccgggatcaaaacccagatggtcgttgagcg

SEQ ID NO: 69 - DNA, 43 bp, synthetic construct
  misc_feature 1..43: note = artificially synthesized sequence
  source 1..43: mol_type = other DNA
gtactgaccacgccgggatcaacacccagatggtcgttgagcg

SEQ ID NO: 70 - DNA, 43 bp, synthetic construct
  misc_feature 1..43: note = artificially synthesized sequence
  source 1..43: mol_type = other DNA
gtactgaccacgccgggatcgtgacccagatggtcgttgagcg

SEQ ID NO: 71 - DNA, 43 bp, synthetic construct
  misc_feature 1..43: note = artificially synthesized sequence
  source 1..43: mol_type = other DNA
gtactgaccacgccgggatcctgacccagatggtcgttgagcg

SEQ ID NO: 72 - DNA, 43 bp, synthetic construct
  misc_feature 1..43: note = artificially synthesized sequence
  source 1..43: mol_type = other DNA
gatcccgccgccgggtgtctatggcagtttgcatatgggtcac

SEQ ID NO: 73 - DNA, 43 bp, synthetic construct
  misc_feature 1..43: note = artificially synthesized sequence
  source 1..43: mol_type = other DNA
gatcccgccgccgggtgtctggggcagtttgcatatgggtcac

SEQ ID NO: 74 - DNA, 43 bp, synthetic construct
  misc_feature 1..43: note = artificially synthesized sequence
  source 1..43: mol_type = other DNA
gatcccgccgccgggtgtctcaggcagtttgcatatgggtcac

SEQ ID NO: 75 - DNA, 43 bp, synthetic construct
  misc_feature 1..43: note = artificially synthesized sequence
  source 1..43: mol_type = other DNA
gatcccgccgccgggtgtcatgggcagtttgcatatgggtcac

SEQ ID NO: 76 - DNA, 43 bp, synthetic construct
  misc_feature 1..43: note = artificially synthesized sequence
  source 1..43: mol_type = other DNA
gatcccgccgccgggtgtcaaaggcagtttgcatatgggtcac

SEQ ID NO: 77 - DNA, 43 bp, synthetic construct
  misc_feature 1..43: note = artificially synthesized sequence
  source 1..43: mol_type = other DNA
gatcccgccgccgggtgtcaacggcagtttgcatatgggtcac

SEQ ID NO: 78 - DNA, 43 bp, synthetic construct
  misc_feature 1..43: note = artificially synthesized sequence
  source 1..43: mol_type = other DNA
gatcccgccgccgggtgtcgtgggcagtttgcatatgggtcac

SEQ ID NO: 79 - DNA, 43 bp, synthetic construct
  misc_feature 1..43: note = artificially synthesized sequence
  source 1..43: mol_type = other DNA
gatcccgccgccgggtgtcctgggcagtttgcatatgggtcac

SEQ ID NO: 80 - DNA, 49 bp, synthetic construct
  misc_feature 1..49: note = artificially synthesized sequence
  source 1..49: mol_type = other DNA
atatgcaaactgccggtgacgttcggagccgggatcatgatgcagaaac

SEQ ID NO: 81 - DNA, 49 bp, synthetic construct
  misc_feature 1..49: note = artificially synthesized sequence
  source 1..49: mol_type = other DNA
atatgcaaactgccggtgacgttcggacccgggatcatgatgcagaaac

SEQ ID NO: 82 - DNA, 49 bp, synthetic construct
  misc_feature 1..49: note = artificially synthesized sequence
  source 1..49: mol_type = other DNA
atatgcaaactgccggtgacagccggcggcgggatcatgatgcagaaac

SEQ ID NO: 83 - DNA, 49 bp, synthetic construct
  misc_feature 1..49: note = artificially synthesized sequence
  source 1..49: mol_type = other DNA
atatgcaaactgccggtgacagccggagccgggatcatgatgcagaaac

SEQ ID NO: 84 - DNA, 49 bp, synthetic construct
  misc_feature 1..49: note = artificially synthesized sequence
  source 1..49: mol_type = other DNA
atatgcaaactgccggtgacagccggacccgggatcatgatgcagaaac

SEQ ID NO: 85 - DNA, 49 bp, synthetic construct
  misc_feature 1..49: note = artificially synthesized sequence
  source 1..49: mol_type = other DNA
atatgcaaactgccggtgacactcggcggcgggatcatgatgcagaaac

SEQ ID NO: 86 - DNA, 49 bp, synthetic construct
  misc_feature 1..49: note = artificially synthesized sequence
  source 1..49: mol_type = other DNA
atatgcaaactgccggtgacactcggagccgggatcatgatgcagaaac

SEQ ID NO: 87 - DNA, 49 bp, synthetic construct
  misc_feature 1..49: note = artificially synthesized sequence
  source 1..49: mol_type = other DNA
atatgcaaactgccggtgacactcggacccgggatcatgatgcagaaac

SEQ ID NO: 88 - DNA, 49 bp, synthetic construct
  misc_feature 1..49: note = artificially synthesized sequence
  source 1..49: mol_type = other DNA
atatgcaaactgccggtgactaccggcggcgggatcatgatgcagaaac

SEQ ID NO: 89 - DNA, 49 bp, synthetic construct
  misc_feature 1..49: note = artificially synthesized sequence
  source 1..49: mol_type = other DNA
atatgcaaactgccggtgactaccggagccgggatcatgatgcagaaac

SEQ ID NO: 90 - DNA, 49 bp, synthetic construct
  misc_feature 1..49: note = artificially synthesized sequence
  source 1..49: mol_type = other DNA
atatgcaaactgccggtgactaccggacccgggatcatgatgcagaaac

SEQ ID NO: 91 - DNA, 49 bp, synthetic construct
  misc_feature 1..49: note = artificially synthesized sequence
  source 1..49: mol_type = other DNA
atatgcaaactgccggtgacacccggcggcgggatcatgatgcagaaac

SEQ ID NO: 92 - DNA, 49 bp, synthetic construct
  misc_feature 1..49: note = artificially synthesized sequence
  source 1..49: mol_type = other DNA
atatgcaaactgccggtgacacccggagccgggatcatgatgcagaaac

SEQ ID NO: 93 - DNA, 49 bp, synthetic construct
  misc_feature 1..49: note = artificially synthesized sequence
  source 1..49: mol_type = other DNA
atatgcaaactgccggtgacacccggacccgggatcatgatgcagaaac

SEQ ID NO: 94 - DNA, 49 bp, synthetic construct
  misc_feature 1..49: note = artificially synthesized sequence
  source 1..49: mol_type = other DNA
atatgcaaactgccggtgacatccggcggcgggatcatgatgcagaaac

SEQ ID NO: 95 - DNA, 49 bp, synthetic construct
  misc_feature 1..49: note = artificially synthesized sequence
  source 1..49: mol_type = other DNA
atatgcaaactgccggtgacatccggagccgggatcatgatgcagaaac

SEQ ID NO: 96 - DNA, 49 bp, synthetic construct
  misc_feature 1..49: note = artificially synthesized sequence
  source 1..49: mol_type = other DNA
atatgcaaactgccggtgacatccggacccgggatcatgatgcagaaac

SEQ ID NO: 97 - DNA, 33 bp, synthetic construct
  misc_feature 1..33: note = artificially synthesized sequence
  source 1..33: mol_type = other DNA
agcgatcccggcgtgagcagtaccgacctgcca

SEQ ID NO: 98 - DNA, 33 bp, synthetic construct
  misc_feature 1..33: note = artificially synthesized sequence
  source 1..33: mol_type = other DNA
agcgatcccggcgtgactagtaccgacctgcca

SEQ ID NO: 99 - DNA, 33 bp, synthetic construct
  misc_feature 1..33: note = artificially synthesized sequence
  source 1..33: mol_type = other DNA
agcgatcccggcgtgtacagtaccgacctgcca

SEQ ID NO: 100 - DNA, 33 bp, synthetic construct
  misc_feature 1..33: note = artificially synthesized sequence
  source 1..33: mol_type = other DNA
agcgatcccggcgtgaccagtaccgacctgcca

SEQ ID NO: 101 - DNA, 43 bp, synthetic construct
  misc_feature 1..43: note = artificially synthesized sequence
  source 1..43: mol_type = other DNA
cgctcaacgaccatctgggtatagatcccggcgtggtcagtac

SEQ ID NO: 102 - DNA, 43 bp, synthetic construct
  misc_feature 1..43: note = artificially synthesized sequence
  source 1..43: mol_type = other DNA
cgctcaacgaccatctgggtccagatcccggcgtggtcagtac

SEQ ID NO: 103 - DNA, 43 bp, synthetic construct
  misc_feature 1..43: note = artificially synthesized sequence
  source 1..43: mol_type = other DNA
cgctcaacgaccatctgggttgagatcccggcgtggtcagtac

SEQ ID NO: 104 - DNA, 43 bp, synthetic construct
  misc_feature 1..43: note = artificially synthesized sequence
  source 1..43: mol_type = other DNA
cgctcaacgaccatctgggtcatgatcccggcgtggtcagtac

SEQ ID NO: 105 - DNA, 43 bp, synthetic construct
  misc_feature 1..43: note = artificially synthesized sequence
  source 1..43: mol_type = other DNA
cgctcaacgaccatctgggttttgatcccggcgtggtcagtac

SEQ ID NO: 106 - DNA, 43 bp, synthetic construct
  misc_feature 1..43: note = artificially synthesized sequence
  source 1..43: mol_type = other DNA
cgctcaacgaccatctgggtgttgatcccggcgtggtcagtac

SEQ ID NO: 107 - DNA, 43 bp, synthetic construct
  misc_feature 1..43: note = artificially synthesized sequence
  source 1..43: mol_type = other DNA
cgctcaacgaccatctgggtcacgatcccggcgtggtcagtac

SEQ ID NO: 108 - DNA, 43 bp, synthetic construct
  misc_feature 1..43: note = artificially synthesized sequence
  source 1..43: mol_type = other DNA
cgctcaacgaccatctgggtcaggatcccggcgtggtcagtac

SEQ ID NO: 109 - DNA, 43 bp, synthetic construct
  misc_feature 1..43: note = artificially synthesized sequence
  source 1..43: mol_type = other DNA
gtgacccatatgcaaactgccatagacacccggcggcgggatc

SEQ ID NO: 110 - DNA, 43 bp, synthetic construct
  misc_feature 1..43: note = artificially synthesized sequence
  source 1..43: mol_type = other DNA
gtgacccatatgcaaactgccccagacacccggcggcgggatc

SEQ ID NO: 111 - DNA, 43 bp, synthetic construct
  misc_feature 1..43: note = artificially synthesized sequence
  source 1..43: mol_type = other DNA
gtgacccatatgcaaactgcctgagacacccggcggcgggatc

SEQ ID NO: 112 - DNA, 43 bp, synthetic construct
  misc_feature 1..43: note = artificially synthesized sequence
  source 1..43: mol_type = other DNA
gtgacccatatgcaaactgcccatgacacccggcggcgggatc

SEQ ID NO: 113 - DNA, 43 bp, synthetic construct
  misc_feature 1..43: note = artificially synthesized sequence
  source 1..43: mol_type = other DNA
gtgacccatatgcaaactgcctttgacacccggcggcgggatc

SEQ ID NO: 114 - DNA, 43 bp, synthetic construct
  misc_feature 1..43: note = artificially synthesized sequence
  source 1..43: mol_type = other DNA
gtgacccatatgcaaactgccgttgacacccggcggcgggatc

SEQ ID NO: 115 - DNA, 43 bp, synthetic construct
  misc_feature 1..43: note = artificially synthesized sequence
  source 1..43: mol_type = other DNA
gtgacccatatgcaaactgcccacgacacccggcggcgggatc

SEQ ID NO: 116 - DNA, 43 bp, synthetic construct
  misc_feature 1..43: note = artificially synthesized sequence
  source 1..43: mol_type = other DNA
gtgacccatatgcaaactgcccaggacacccggcggcgggatc

SEQ ID NO: 117 - DNA, 98 bp, synthetic construct
  misc_feature 1..98: note = artificially synthesized sequence
  source 1..98: mol_type = other DNA
ggcgtaatacgactcactatagcgtccgtagctcagttggttagagcaccaccttgacatggtgggggtcggtggttcgagtccactcggacgcacca

SEQ ID NO: 118 - RNA, 77 nt, synthetic construct
  misc_feature 1..77: note = artificially synthesized sequence
  source 1..77: mol_type = other RNA
gcgtccgtagctcagttggttagagcaccaccttgacatggtgggggtcggtggttcgagtccactcggacgcacca

SEQ ID NO: 119 - DNA, 92 bp, synthetic construct
  misc_feature 1..92: note = artificially synthesized sequence
  source 1..92: mol_type = other DNA
ggcgtaatacgactcactatagggttaactttaacaaggagaaaaacatgcgtgtccgtgactacaaggacgacgacgacaagtaagcttcg

SEQ ID NO: 120 - RNA, 71 nt, synthetic construct
  misc_feature 1..71: note = artificially synthesized sequence
  source 1..71: mol_type = other RNA
gggttaactttaacaaggagaaaaacatgcgtgtccgtgactacaaggacgacgacgacaagtaagcttcg

SEQ ID NO: 121 - DNA, 95 bp, synthetic construct
  misc_feature 1..95: note = artificially synthesized sequence
  source 1..95: mol_type = other DNA
ggcgtaatacgactcactatagggttaactttaacaaggagaaaaacatgcgtgtcgtccgtgactacaaggacgacgacgacaagtaagcttcg

SEQ ID NO: 122 - RNA, 74 nt, synthetic construct
  misc_feature 1..74: note = artificially synthesized sequence
  source 1..74: mol_type = other RNA
gggttaactttaacaaggagaaaaacatgcgtgtcgtccgtgactacaaggacgacgacgacaagtaagcttcg

SEQ ID NO: 123 - DNA, 98 bp, synthetic construct
  misc_feature 1..98: note = artificially synthesized sequence
  source 1..98: mol_type = other DNA
ggcgtaatacgactcactatagggttaactttaacaaggagaaaaacatgcgtgtcgtcgtccgtgactacaaggacgacgacgacaagtaagcttcg

SEQ ID NO: 124 - RNA, 77 nt, synthetic construct
  misc_feature 1..77: note = artificially synthesized sequence
  source 1..77: mol_type = other RNA
gggttaactttaacaaggagaaaaacatgcgtgtcgtcgtccgtgactacaaggacgacgacgacaagtaagcttcg

SEQ ID NO: 125 - protein, 12 aa, synthetic construct
  REGION 1..12: note = artificially synthesized sequence
  SITE 3: note = MISC_FEATURE - Xaa is valine or methyl valine
MRXRDYKDDDDK

SEQ ID NO: 126 - protein, 13 aa, synthetic construct
  REGION 1..13: note = artificially synthesized sequence
  REGION 3..4: note = MISC_FEATURE - Xaa is valine or methyl valine
MRXXRDYKDDDDK

SEQ ID NO: 127 - protein, 14 aa, synthetic construct
  REGION 1..14: note = artificially synthesized sequence
  REGION 3..5: note = MISC_FEATURE - Xaa is valine or methyl valine
MRXXXRDYKDDDDK

SEQ ID NO: 128 - DNA, 49 bp, synthetic construct
  misc_feature 1..49: note = artificially synthesized sequence
  source 1..49: mol_type = other DNA
gtaactatgcgctgatcccagctgcagaagttccgctgactaacctggt

SEQ ID NO: 129 - DNA, 49 bp, synthetic construct
  misc_feature 1..49: note = artificially synthesized sequence
  source 1..49: mol_type = other DNA
gtaactatgcgctgatcccaagtgcagaagttccgctgactaacctggt

SEQ ID NO: 130 - DNA, 49 bp, synthetic construct
  misc_feature 1..49: note = artificially synthesized sequence
  source 1..49: mol_type = other DNA
gtaactatgcgctgatcccaggtgcagaagttccgctgactaacctggt

SEQ ID NO: 131 - DNA, 49 bp, synthetic construct
  misc_feature 1..49: note = artificially synthesized sequence
  source 1..49: mol_type = other DNA
gtaactatgcgctgatcccaacggcagctgttccgctgactaacctggt

SEQ ID NO: 132 - DNA, 49 bp, synthetic construct
  misc_feature 1..49: note = artificially synthesized sequence
  source 1..49: mol_type = other DNA
gtaactatgcgctgatcccaacggcagtagttccgctgactaacctggt

SEQ ID NO: 133 - DNA, 49 bp, synthetic construct
  misc_feature 1..49: note = artificially synthesized sequence
  source 1..49: mol_type = other DNA
gtaactatgcgctgatcccaacggcaagtgttccgctgactaacctggt

SEQ ID NO: 134 - DNA, 33 bp, synthetic construct
  misc_feature 1..33: note = artificially synthesized sequence
  source 1..33: mol_type = other DNA
ctggttcataccctggctggttctggtctggct

SEQ ID NO: 135 - DNA, 49 bp, synthetic construct
  misc_feature 1..49: note = artificially synthesized sequence
  source 1..49: mol_type = other DNA
gtaactatgcgctgatcccagctgcagctgttccgctgactaacctggt

SEQ ID NO: 136 - DNA, 49 bp, synthetic construct
  misc_feature 1..49: note = artificially synthesized sequence
  source 1..49: mol_type = other DNA
gtaactatgcgctgatcccagctgcagtagttccgctgactaacctggt

SEQ ID NO: 137 - DNA, 49 bp, synthetic construct
  misc_feature 1..49: note = artificially synthesized sequence
  source 1..49: mol_type = other DNA
gtaactatgcgctgatcccagctgcaagtgttccgctgactaacctggt

SEQ ID NO: 138 - DNA, 49 bp, synthetic construct
  misc_feature 1..49: note = artificially synthesized sequence
  source 1..49: mol_type = other DNA
gtaactatgcgctgatcccaagtgcagctgttccgctgactaacctggt

SEQ ID NO: 139 - DNA, 49 bp, synthetic construct
  misc_feature 1..49: note = artificially synthesized sequence
  source 1..49: mol_type = other DNA
gtaactatgcgctgatcccaagtgcagtagttccgctgactaacctggt

SEQ ID NO: 140 - DNA, 49 bp, synthetic construct
  misc_feature 1..49: note = artificially synthesized sequence
  source 1..49: mol_type = other DNA
gtaactatgcgctgatcccaagtgcaagtgttccgctgactaacctggt

SEQ ID NO: 141 - DNA, 49 bp, synthetic construct
  misc_feature 1..49: note = artificially synthesized sequence
  source 1..49: mol_type = other DNA
gtaactatgcgctgatcccaggtgcagctgttccgctgactaacctggt

SEQ ID NO: 142 - DNA, 49 bp, synthetic construct
  misc_feature 1..49: note = artificially synthesized sequence
  source 1..49: mol_type = other DNA
gtaactatgcgctgatcccaggtgcagtagttccgctgactaacctggt

SEQ ID NO: 143 - DNA, 49 bp, synthetic construct
  misc_feature 1..49: note = artificially synthesized sequence
  source 1..49: mol_type = other DNA
gtaactatgcgctgatcccaggtgcaagtgttccgctgactaacctggt

SEQ ID NO: 144 - DNA, 33 bp, synthetic construct
  misc_feature 1..33: note = artificially synthesized sequence
  source 1..33: mol_type = other DNA
cagttcgacaaagttgctatggtgcagatcgtg

SEQ ID NO: 145 - DNA, 33 bp, synthetic construct
  misc_feature 1..33: note = artificially synthesized sequence
  source 1..33: mol_type = other DNA
atgcaccagttcgacgctgttgaaatggtgcag

SEQ ID NO: 146 - DNA, 49 bp, synthetic construct
  misc_feature 1..49: note = artificially synthesized sequence
  source 1..49: mol_type = other DNA
gtaactatgcgctgatcccaacggcaggtgttccgctgactaacctggt

SEQ ID NO: 147 - DNA, 49 bp, synthetic construct
  misc_feature 1..49: note = artificially synthesized sequence
  source 1..49: mol_type = other DNA
gtaactatgcgctgatcccaacggcagacgttccgctgactaacctggt

SEQ ID NO: 148 - DNA, 49 bp, synthetic construct
  misc_feature 1..49: note = artificially synthesized sequence
  source 1..49: mol_type = other DNA
gtaactatgcgctgatcccaagtgcaggtgttccgctgactaacctggt

SEQ ID NO: 149 - DNA, 49 bp, synthetic construct
  misc_feature 1..49: note = artificially synthesized sequence
  source 1..49: mol_type = other DNA
gtaactatgcgctgatcccaagtgcagacgttccgctgactaacctggt

SEQ ID NO: 150 - DNA, 49 bp, synthetic construct
  misc_feature 1..49: note = artificially synthesized sequence
  source 1..49: mol_type = other DNA
accaggttagtcagcggaacttctgcagctgggatcagcgcatagttac

SEQ ID NO: 151 - DNA, 49 bp, synthetic construct
  misc_feature 1..49: note = artificially synthesized sequence
  source 1..49: mol_type = other DNA
accaggttagtcagcggaacttctgcacttgggatcagcgcatagttac

SEQ ID NO: 152 - DNA, 49 bp, synthetic construct
  misc_feature 1..49: note = artificially synthesized sequence
  source 1..49: mol_type = other DNA
accaggttagtcagcggaacttctgcacctgggatcagcgcatagttac

SEQ ID NO: 153 - DNA, 49 bp, synthetic construct
  misc_feature 1..49: note = artificially synthesized sequence
  source 1..49: mol_type = other DNA
accaggttagtcagcggaacagctgccgttgggatcagcgcatagttac

SEQ ID NO: 154 - DNA, 49 bp, synthetic construct
  misc_feature 1..49: note = artificially synthesized sequence
  source 1..49: mol_type = other DNA
accaggttagtcagcggaactactgccgttgggatcagcgcatagttac

SEQ ID NO: 155 - DNA, 49 bp, synthetic construct
  misc_feature 1..49: note = artificially synthesized sequence
  source 1..49: mol_type = other DNA
accaggttagtcagcggaacacttgccgttgggatcagcgcatagttac

SEQ ID NO: 156 - DNA, 33 bp, synthetic construct
  misc_feature 1..33: note = artificially synthesized sequence
  source 1..33: mol_type = other DNA
agccagaccagaaccagccagggtatgaaccag

SEQ ID NO: 157 - DNA, 49 bp, synthetic construct
  misc_feature 1..49: note = artificially synthesized sequence
  source 1..49: mol_type = other DNA
accaggttagtcagcggaacagctgcagctgggatcagcgcatagttac

SEQ ID NO: 158 - DNA, 49 bp, synthetic construct
  misc_feature 1..49: note = artificially synthesized sequence
  source 1..49: mol_type = other DNA
accaggttagtcagcggaactactgcagctgggatcagcgcatagttac

SEQ ID NO: 159 - DNA, 49 bp, synthetic construct
  misc_feature 1..49: note = artificially synthesized sequence
  source 1..49: mol_type = other DNA
accaggttagtcagcggaacacttgcagctgggatcagcgcatagttac

SEQ ID NO: 160 - DNA, 49 bp, synthetic construct
  misc_feature 1..49: note = artificially synthesized sequence
  source 1..49: mol_type = other DNA
accaggttagtcagcggaacagctgcacttgggatcagcgcatagttac

SEQ ID NO: 161 - DNA, 49 bp, synthetic construct
  misc_feature 1..49: note = artificially synthesized sequence
  source 1..49: mol_type = other DNA
accaggttagtcagcggaactactgcacttgggatcagcgcatagttac

SEQ ID NO: 162 - DNA, 49 bp, synthetic construct
  misc_feature 1..49: note = artificially synthesized sequence
  source 1..49: mol_type = other DNA
accaggttagtcagcggaacacttgcacttgggatcagcgcatagttac

SEQ ID NO: 163 - DNA, 49 bp, synthetic construct
  misc_feature 1..49: note = artificially synthesized sequence
  source 1..49: mol_type = other DNA
accaggttagtcagcggaacagctgcacctgggatcagcgcatagttac

SEQ ID NO: 164 - DNA, 49 bp, synthetic construct
  misc_feature 1..49: note = artificially synthesized sequence
  source 1..49: mol_type = other DNA
accaggttagtcagcggaactactgcacctgggatcagcgcatagttac

SEQ ID NO: 165 - DNA, 49 bp, synthetic construct
  misc_feature 1..49: note = artificially synthesized sequence
  source 1..49: mol_type = other DNA
accaggttagtcagcggaacacttgcacctgggatcagcgcatagttac

SEQ ID NO: 166 - DNA, 33 bp, synthetic construct
  misc_feature 1..33: note = artificially synthesized sequence
  source 1..33: mol_type = other DNA
cacgatctgcaccatagcaactttgtcgaactg

SEQ ID NO: 167 - DNA, 33 bp, synthetic construct
  misc_feature 1..33: note = artificially synthesized sequence
  source 1..33: mol_type = other DNA
ctgcaccatttcaacagcgtcgaactggtgcat

SEQ ID NO: 168 - DNA, 49 bp, synthetic construct
  misc_feature 1..49: note = artificially synthesized sequence
  source 1..49: mol_type = other DNA
accaggttagtcagcggaacacctgccgttgggatcagcgcatagttac

SEQ ID NO: 169 - DNA, 49 bp, synthetic construct
  misc_feature 1..49: note = artificially synthesized sequence
  source 1..49: mol_type = other DNA
accaggttagtcagcggaacgtctgccgttgggatcagcgcatagttac

SEQ ID NO: 170 - DNA, 49 bp, synthetic construct
  misc_feature 1..49: note = artificially synthesized sequence
  source 1..49: mol_type = other DNA
accaggttagtcagcggaacacctgcacttgggatcagcgcatagttac

SEQ ID NO: 171 - DNA, 49 bp, synthetic construct
  misc_feature 1..49: note = artificially synthesized sequence
  source 1..49: mol_type = other DNA
accaggttagtcagcggaacgtctgcacttgggatcagcgcatagttac

SEQ ID NO: 172 - DNA, 114 bp, synthetic construct
  misc_feature 1..114: note = artificially synthesized sequence
  source 1..114: mol_type = other DNA
ggcgtaatacgactcactataggtgaggtggccgagaggctgaaggcgctcccctgctaagggagtatgcggtcaaaagctgcatccggggttcgaatccccgcctcaccgcca

SEQ ID NO: 173 - RNA, 93 nt, synthetic construct
  misc_feature 1..93: note = artificially synthesized sequence
  source 1..93: mol_type = other RNA
ggtgaggtggccgagaggctgaaggcgctcccctgctaagggagtatgcggtcaaaagctgcatccggggttcgaatccccgcctcaccgcca

SEQ ID NO: 174 - DNA, 92 bp, synthetic construct
  misc_feature 1..92: note = artificially synthesized sequence
  source 1..92: mol_type = other DNA
ggcgtaatacgactcactatagggttaactttaacaaggagaaaaacatgcgttcccgtgactacaaggacgacgacgacaagtaagcttcg

SEQ ID NO: 175 - RNA, 71 nt, synthetic construct
  misc_feature 1..71: note = artificially synthesized sequence
  source 1..71: mol_type = other RNA
gggttaactttaacaaggagaaaaacatgcgttcccgtgactacaaggacgacgacgacaagtaagcttcg

SEQ ID NO: 176 - protein, 12 aa, synthetic construct
  REGION 1..12: note = artificially synthesized sequence
  SITE 3: note = MISC_FEATURE - Xaa is serine or methyl serine
MRXRDYKDDDDK

SEQ ID NO: 177 - DNA, 103 bp, synthetic construct
  misc_feature 1..103: note = artificially synthesized sequence
  source 1..103: mol_type = other DNA
ggcgtaatacgactcactatagggttaactttaagaaggagatatacatatgaaggctggtccgggttttatgactaagagtggtagtggtagttaagcttcg

SEQ ID NO: 178 - RNA, 82 nt, synthetic construct
  misc_feature 1..82: note = artificially synthesized sequence
  source 1..82: mol_type = other RNA
gggttaactttaagaaggagatatacatatgaaggctggtccgggttttatgactaagagtggtagtggtagttaagcttcg

SEQ ID NO: 179 - protein, 15 aa, synthetic construct
  REGION 1..15: note = artificially synthesized sequence
  SITE 9: note = MISC_FEATURE - Xaa is threonine or methyl threonine
MKAGPGFMXKSGSGS

SEQ ID NO: 180 - protein, 7 aa, synthetic construct
  REGION 1..7: note = artificially synthesized sequence
  SITE 4: note = MISC_FEATURE - Xaa is Asn (N), Tyr (Y), or Thr (T).
  SITE 5: note = MISC_FEATURE - Xaa is any amino acid.
  SITE 6: note = MISC_FEATURE - Xaa is Thr (T) or Ser (S).
PPPXXXG

SEQ ID NO: 181 - protein, 7 aa, synthetic construct
  REGION 1..7: note = artificially synthesized sequence
  SITE 5: note = MISC_FEATURE - Xaa is any amino acid.
PPPNXTG

SEQ ID NO: 182 - protein, 951 aa, synthetic construct
  REGION 1..951: note = artificially synthesized sequence
MEKTYNPQDIEQPLYEHWEKQGYFKPNGDESQESFCIMIPPPGVSGSLHMGHAFQQTIMDTMIRYQRMQGKNTLWQVGTDHAGIATQMVVERKIAAEEGKTRHDYGREAFIDKIWEWKAESGGTITRQMRRLGNSVDWERERFTMDEGLSNAVKEVFVRLYKEDLIYRGKRLVNWDPKLRTAISDLEVENRESKGSMWHIRYPLADGAKTADGKDYLVVATTRPETLLGDTGVAVNPEDPRYKDLIGKYVILPLVNRRIPIVGDEHADMEKGTGCVKIAPAHDFNDYEVGKRHALPMINILTFDGDIRESAQVFDTKGNESDVYSSEIPAEFQKLERFAARKAVVAAVDALGLLEEIKPHDLTVPYGDRGGVVIEPMLTDQWYVRADVLAKPAVEAVENGDIQFVPKQYENMYFSWMRDIQDWCISRQLWWGHRIPAWYDEAGNVYVGRNEDEVRKENNLGADVVLRQDEDVLDTWFSSALWTFSTLGWPENTDALRQFHPTSVMVSGFDIIFFWIARMIMMTMHFIKDENGKPQVPFHTVYMTGLIRDDEGQKMSKSKGNVIDPLDMVDGISLPELLEKRTGNMMQPQLADKIRKRTEKQFPNGIEPHGTDALRFTLAALASTGRDINWDMKRLEGYRNFCNKLWNASRFVLMNTEGQDCGFNGGEMTLSLADRWILAEFNQTIKAYREALDSFRFDIAAGILYEFTWNQFCDWYLELTKPVMNGGTEAELRGTRHTLVTVLEGLLRLAHPIIPFITETIWQRVKVLCGITADTIMLQPFPQYDASQVDEAALADTEWLKQAIVAVRNIRAEMNIAPGKPLELLLRGCSADAERRVNENRGFLQTLARLESITVLPADDKGPVSVTKIIDGAELLIPMAGLINKEDELARLAKEVAKIEGEISRIENKLANEGFVARAPEAVIAKEREKLEGYAEAKAKLIEQQAVIAAL

SEQ ID NO: 183 - protein, 951 aa, synthetic construct
  REGION 1..951: note = artificially synthesized sequence
MEKTYNPQDIEQPLYEHWEKQGYFKPNGDESQESFCIMIPPPGVSGSLHMGHAFQQTIMDTMIRYQRMQGKNTLWQVGTDHAGIATQMVVERKIAAEEGKTRHDYGREAFIDKIWEWKAESGGTITRQMRRLGNSVDWERERFTMDEGLSNAVKEVFVRLYKEDLIYRGKRLVNWDPKLRTAISDLEVENRESKGSMWHIRYPLADGAKTADGKDYLVVATTRPETLLGDTGVAVNPEDPRYKDLIGKYVILPLVNRRIPIVGDEHADMEKGTGCVKIGPAHDFNDYEVGKRHALPMINILTFDGDIRESAQVFDTKGNESDVYSSEIPAEFQKLERFAARKAVVAAVDALGLLEEIKPHDLTVPYGDRGGVVIEPMLTDQWYVRADVLAKPAVEAVENGDIQFVPKQYENMYFSWMRDIQDWCISRQLWWGHRIPAWYDEAGNVYVGRNEDEVRKENNLGADVVLRQDEDVLDTWFSSALWTFSTLGWPENTDALRQFHPTSVMVSGFDIIFFWIARMIMMTMHFIKDENGKPQVPFHTVYMTGLIRDDEGQKMSKSKGNVIDPLDMVDGISLPELLEKRTGNMMQPQLADKIRKRTEKQFPNGIEPHGTDALRFTLAALASTGRDINWDMKRLEGYRNFCNKLWNASRFVLMNTEGQDCGFNGGEMTLSLADRWILAEFNQTIKAYREALDSFRFDIAAGILYEFTWNQFCDWYLELTKPVMNGGTEAELRGTRHTLVTVLEGLLRLAHPIIPFITETIWQRVKVLCGITADTIMLQPFPQYDASQVDEAALADTEWLKQAIVAVRNIRAEMNIAPGKPLELLLRGCSADAERRVNENRGFLQTLARLESITVLPADDKGPVSVTKIIDGAELLIPMAGLINKEDELARLAKEVAKIEGEISRIENKLANEGFVARAPEAVIAKEREKLEGYAEAKAKLIEQQAVIAAL

SEQ ID NO: 184 - protein, 334 aa, synthetic construct
  REGION 1..334: note = artificially synthesized sequence
MTKPIVFSGAQPSGELTIGNYMGALRQWVNMQDDYHCIYCIVDQHAITVRQDAQKLRKATLDTLALYLACGIDPEKSTIFVQSHVPEHAQLGWALNCYTYFGELSRMTQFKDKSARYAENINAGLFDYPVLMAADILLYQTNLVPVGEDAKQALELSRDIAQRFNALYGEIFKVPEPFIPKSGARVMSLLEPTKKMSKSDDNRNNVIGLLEDPKSVVKKIKRAVTDSDEPPVVRYDVQNKAGVSNLLDILSAVTGQSIPELEKQFEGKMYGHLKGEVADAVSGMLTELQERYHRFRNDEAFLQQVMKDGAEKASAHASRTLKAVYEAIGFVAKP

SEQ ID NO: 185 - protein, 334 aa, synthetic construct
  REGION 1..334: note = artificially synthesized sequence
MTKPIVFSGAQPSGELTIGNYMGALRQWVNMQDDYHCIYCIVDQHAITVRQDAQKLRKATLDTLALYLACGIDPEKSTIFVQSHVPEHAQLGWALNCYTYFGELSRMTQFKDKSARYAENINAGLFDYPVLAAADILLYQTNLVPVGEDQKQHLELSRDIAQRFNALYGEIFKVPEPFIPKSGARVMSLLEPTKKMSKSDDNRNNVIGLLEDPKSVVKKIKRAVTDSDEPPVVRYDVQNKAGVSNLLDILSAVTGQSIPELEKQFEGKMYGHLKGEVADAVSGMLTELQERYHRFRNDEAFLQQVMKDGAEKASAHASRTLKAVYEAIGFVAKP

SEQ ID NO: 186 - protein, 334 aa, synthetic construct
  REGION 1..334: note = artificially synthesized sequence
MTKPIVFSGAQPSGELTIGNYMGALRQWVNMQDDYHCIYCIVDQHAITVRQDAQKLRKATLDTLALYLACGIDPEKSTIFVQSHVPEHAQLGWALNCYTYFGELSRMTQFKDKSARYAENINAGLFDYPVLVAADILLYQTNLVPVGEDAKQALELSRDIAQRFNALYGEIFKVPEPFIPKSGARVMSLLEPTKKMSKSDDNRNNVIGLLEDPKSVVKKIKRAVTDSDEPPVVRYDVQNKAGVSNLLDILSAVTGQSIPELEKQFEGKMYGHLKGEVADAVSGMLTELQERYHRFRNDEAFLQQVMKDGAEKASAHASRTLKAVYEAIGFVAKP

SEQ ID NO: 187 - protein, 860 aa, synthetic construct
  REGION 1..860: note = artificially synthesized sequence
MQEQYRPEEIESKVQLHWDEKRTFEVTEDESKEKYYCLSMLPGPSGRLHMGHVRNYTIGDVIARYQRMLGKNVLQPIGWDAFGLPAEGAAVKNNTAPAPWTYDNIAYMKNQLKMLGFGYDWSRELATCTPEYYRWEQKFFTELYKKGLVYKKTSAVNWCPNDQTVLANEQVIDGCCWRCDTKVERKEIPQWFIKITAYADELLNDLDKLDHWPDTVKTMQRNWIGRSEGVEITFNVNDYDNTLTVYTTRPDTFMGCTYLAVAAGHPLAQKAAENNPELAAFIDECRNTKVAEAEMATMEKKGVDTGFKAVHPLTGEEIPVWAANFVLMEYGTGAVMAVPGHDQRDYEFASKYGLNIKPVILAADGSEPDLSQQALTEKGVLFNSGEFNGLDHEAAFNAIADKLTAMGVGERKVNYRLRDWGVSRQRYWGAPIPMVTLEDGTVMPTPDDQLPVILPEDVVMDGITSPIKADPEWAKTTVNGMPALRETDTFDTFMESSWYYARYTCPQYKEGMLDSEAANYWLPVDIYIGGIEHAIMHLLYFRFFHKLMRDAGMVNSDEPAKQLLCQGMVLADAFYYVGENGERNWVSPVDAIVERDEKGRIVKAKDAAGHELVYTGMSKMSKSKNNGIDPQVMVERYGADTVRLFMMFASPADMTLEWQESGVEGANRFLKRVWKLVYEHTAKGDVAALNVDALTENQKALRRDVHKTIAKVTDDIGRRQTFNTAIAAIMELMNKLAKAPTDGEQDRALMQEALLAVVRMLNPFTPHICFTLWQELKGEGDIDNAPWPVADEKAMVEDSTLVVVQVNGKVRAKITVPVDATEEQVRERAGQEHLVAKYLDGVTVRKVIYVPGKLLNLVVG

SEQ ID NO: 188 - protein, 334 aa, synthetic construct
MTKPIVFSGAQPSGELTIGNYMGALRQWVNMQDDYHCIYCIVDQHAITVRQDAQKLRKATLDTLALYLACGIDPEKSTIFVQSHVPEHAQLGWALNCYTYFGELSRMTQFKDKSARYAENINAGLFDYPVLMAADILLYQTNLVPVGEDQKQHLELSRDIAQRFNALYGEIFKVPEPFIPKSGARVMSLLEPTKKMSKSDDNRNNVIGLLEDPKSVVKKIKRAVTDSDEPPVVRYDVQNKAGVSNLLDILSAVTGQSIPELEKQFEGKMYGHLKGEVADAVSGMLTELQERYHRFRNDEAFLQQVMKDGAEKASAHASRTLKAVYEAIGFVAKP

SEQ ID NO: 189 - protein, 860 aa, synthetic construct [sequence omitted]

SEQ ID NO: 190 - DNA, 2853 bp, synthetic construct
  misc_feature 1..2853: note = artificially synthesized sequence
  source 1..2853: mol_type = other DNA
atggaaaaaacctataatccgcaagacattgaacaaccgctgtatgaacactgggaaaaacaaggctactttaaaccgaacggcgatgaaagtcaggaatccttttgcattatgattccgccgccgggcgtgtcaggttcgctgcatatgggccacgcgttccagcaaacgattatggataccatgatccgttatcagcgcatgcaaggtaaaaacacgctgtggcaggttggcaccgatcatgcaggtatcgctacgcaaatggtggttgaacgtaaaattgcggccgaagaaggcaaaacccgtcacgactacggtcgcgaagcgtttattgataaaatctgggaatggaaagccgaaagtggcggtaccattacgcgtcagatgcgtcgcctgggtaactccgtggactgggaacgtgaacgctttaccatggatgaaggtctgagcaatgcagttaaagaagttttcgtccgtctgtataaagaagatctgatctaccgtggcaaacgcctggtgaactgggacccgaaactgcgcaccgccatttctgatctggaagttgaaaatcgtgaatcaaaaggttcgatgtggcatatccgctatccgctggcggatggcgccaaaaccgcagatggtaaagactacctggtcgtggcaaccacgcgtccggaaacgctgctgggcgataccggtgtggccgttaacccggaagacccgcgctataaagatctgattggcaaatacgtgatcctgccgctggttaatcgtcgcattccgatcgtgggtgacgaacacgcggatatggaaaaaggcaccggttgcgttaaaattgcgccggcccatgattttaacgactatgaagtcggcaaacgtcacgccctgccgatgattaatatcctgacgtttgatggcgacattcgcgaaagtgcacaggtcttcgacaccaagggtaatgaatccgatgtgtacagctctgaaatcccggcggaatttcaaaaactggaacgtttcgcagctcgcaaagcagttgtcgcagcagtcgatgcactgggtctgctggaagaaattaaaccgcatgacctgacggttccgtatggtgatcgtggcggtgtggttatcgaaccgatgctgaccgaccagtggtatgtgcgcgccgatgtcctggctaaaccggcggtcgaagccgtggaaaacggtgatattcagtttgtgccgaaacaatacgaaaacatgtacttctcatggatgcgtgatattcaggactggtgtatctcgcgtcaactgtggtggggccatcgcattccggcatggtatgatgaagctggcaacgtctacgtgggtcgtaatgaagacgaagtgcgcaaagaaaacaatctgggtgcggatgtcgtgctgcgccaggatgaagacgttctggacacctggtttagttccgcactgtggaccttcagtacgctgggctggccggaaaacacggatgcgctgcgtcagtttcacccgaccagcgttatggtctctggtttcgacattatctttttctggattgcccgcatgatcatgatgaccatgcatttcatcaaagatgaaaacggcaaaccgcaggtcccgttccacacggtgtatatgaccggcctgatccgtgatgacgaaggtcaaaaaatgagcaaatctaaaggcaacgttatcgatccgctggacatggtcgatggtattagcctgccggaactgctggaaaaacgcacgggtaacatgatgcagccgcaactggcggataaaatccgtaaacgcaccgaaaaacagtttccgaatggcattgaaccgcatggtacggatgcgctgcgtttcaccctggcagctctggcatcaaccggtcgtgacattaattgggatatgaaacgcctggaaggttatcgcaacttttgcaataaactgtggaacgcctcacgtttcgtgctgatgaatacggaaggccaggattgtggttttaacggcggtgaaatgaccctgtcgctggcagaccgctggatcctggctgaattcaatcaaacgattaaagcgtaccgtgaagccctggactcttttcgcttcgatattgcggccggcatcctgtatgaatttacctggaaccagttctgcgattggtacctggaactgacgaaaccggtgatgaatggcggtaccgaagcggaactgcgtggcacccgccatacgctggtgaccgttctggaaggtctgctgcgtctggcccacccgattatcccgtttattaccgaaacgatctggcagcgcgtcaaagtgctgtgtggtattacggcggataccatcatgctgcagccgttcccgcaatatgacgcaagtcaggttgatgaagcagctctggctgataccgaatggctgaaacaagcaattgttgctgtccgtaacatccgcgcggaaatgaatattgccccgggcaaaccgctggaactgctgctgcgtggttgttccgcagatgctgaacgtcgcgtgaacgaaaatcgtggctttctgcagacgctggcacgcctggaaagcattaccgttctgccggctgatgacaaaggtccggtgtctgttaccaaaattatcgatggcgcagaactgctgatcccgatggctggtctgattaacaaagaagacgaactggcgcgtctggccaaagaagtggcaaaaattgaaggcgaaattagccgcatcgaaaacaaactggctaatgaaggttttgtggcgcgtgccccggaagcagttatcgctaaagaacgcgaaaaactggaaggttacgcagaagcaaaagccaaactgattgaacaacaagcggttatcgcagcactg

SEQ ID NO: 191 - DNA, 2853 bp, synthetic construct
  misc_feature 1..2853: note = artificially synthesized sequence
  source 1..2853: mol_type = other DNA
atggaaaaaacctataatccgcaagacattgaacaaccgctgtatgaacactgggaaaaacaaggctactttaaaccgaacggcgatgaaagtcaggaatccttttgcattatgattccgccgccgggcgtgtcaggttcgctgcatatgggccacgcgttccagcaaacgattatggataccatgatccgttatcagcgcatgcaaggtaaaaacacgctgtggcaggttggcaccgatcatgcaggtatcgctacgcaaatggtggttgaacgtaaaattgcggccgaagaaggcaaaacccgtcacgactacggtcgcgaagcgtttattgataaaatctgggaatggaaagccgaaagtggcggtaccattacgcgtcagatgcgtcgcctgggtaactccgtggactgggaacgtgaacgctttaccatggatgaaggtctgagcaatgcagttaaagaagttttcgtccgtctgtataaagaagatctgatctaccgtggcaaacgcctggtgaactgggacccgaaactgcgcaccgccatttctgatctggaagttgaaaatcgtgaatcaaaaggttcgatgtggcatatccgctatccgctggcggatggcgccaaaaccgcagatggtaaagactacctggtcgtggcaaccacgcgtccggaaacgctgctgggcgataccggtgtggccgttaacccggaagacccgcgctataaagatctgattggcaaatacgtgatcctgccgctggttaatcgtcgcattccgatcgtgggtgacgaacacgcggatatggaaaaaggcaccggttgcgttaaaattggtccggcccatgattttaacgactatgaagtcggcaaacgtcacgccctgccgatgattaatatcctgacgtttgatggcgacattcgcgaaagtgcacaggtcttcgacaccaagggtaatgaatccgatgtgtacagctctgaaatcccggcggaatttcaaaaactggaacgtttcgcagctcgcaaagcagttgtcgcagcagtcgatgcactgggtctgctggaagaaattaaaccgcatgacctgacggttccgtatggtgatcgtggcggtgtggttatcgaaccgatgctgaccgaccagtggtatgtgcgcgccgatgtcctggctaaaccggcggtcgaagccgtggaaaacggtgatattcagtttgtgccgaaacaatacgaaaacatgtacttctcatggatgcgtgatattcaggactggtgtatctcgcgtcaactgtggtggggccatcgcattccggcatggtatgatgaagctggcaacgtctacgtgggtcgtaatgaagacgaagtgcgcaaagaaaacaatctgggtgcggatgtcgtgctgcgccaggatgaagacgttctggacacctggtttagttccgcactgtggaccttcagtacgctgggctggccggaaaacacggatgcgctgcgtcagtttcacccgaccagcgttatggtctctggtttcgacattatctttttctggattgcccgcatgatcatgatgaccatgcatttcatcaaagatgaaaacggcaaaccgcaggtcccgttccacacggtgtatatgaccggcctgatccgtgatgacgaaggtcaaaaaatgagcaaatctaaaggcaacgttatcgatccgctggacatggtcgatggtattagcctgccggaactgctggaaaaacgcacgggtaacatgatgcagccgcaactggcggataaaatccgtaaacgcaccgaaaaacagtttccgaatggcattgaaccgcatggtacggatgcgctgcgtttcaccctggcagctctggcatcaaccggtcgtgacattaattgggatatgaaacgcctggaaggttatcgcaacttttgcaataaactgtggaacgcctcacgtttcgtgctgatgaatacggaaggccaggattgtggttttaacggcggtgaaatgaccctgtcgctggcagaccgctggatcctggctgaattcaatcaaacgattaaagcgtaccgtgaagccctggactcttttcgcttcgatattgcggccggcatcctgtatgaatttacctggaaccagttctgcgattggtacctggaactgacgaaaccggtgatgaatggcggtaccgaagcggaactgcgtggcacccgccatacgctggtgaccgttctggaaggtctgctgcgtctggcccacccgattatcccgtttattaccgaaacgatctggcagcgcgtcaaagtgctgtgtggtattacggcggataccatcatgctgcagccgttcccgcaatatgacgcaagtcaggttgatgaagcagctctggctgataccgaatggctgaaacaagcaattgttgctgtccgtaacatccgcgcggaaatgaatattgccccgggcaaaccgctggaactgctgctgcgtggttgttccgcagatgctgaacgtcgcgtgaacgaaaatcgtggctttctgcagacgctggcacgcctggaaagcattaccgttctgccggctgatgacaaaggtccggtgtctgttaccaaaattatcgatggcgcagaactgctgatcccgatggctggtctgattaacaaagaagacgaactggcgcgtctggccaaagaagtggcaaaaattgaaggcgaaattagccgcatcgaaaacaaactggctaatgaaggttttgtggcgcgtgccccggaagcagttatcgctaaagaacgcgaaaaactggaaggttacgcagaagcaaaagccaaactgattgaacaacaagcggttatcgcagcactg

SEQ ID NO: 192 - DNA, 1002 bp, synthetic construct
  misc_feature 1..1002: note = artificially synthesized sequence
  source 1..1002: mol_type = other DNA
atgacgaaaccgattgtcttctctggtgctcaaccgtctggtgaactgacgattggtaactacatgggtgctctgcgccagtgggtgaacatgcaggatgactatcattgcatttactgtatcgtggatcaacacgccattaccgttcgtcaggacgctcaaaaactgcgcaaagcgaccctggatacgctggccctgtatctggcatgcggtattgacccggaaaaatcaacgatctttgtgcagtcgcatgttccggaacacgctcaactgggttgggcgctgaactgttatacctactttggcgaactgtcacgtatgacgcagttcaaagataaatcggctcgctatgcggaaaacattaatgcgggtctgtttgattacccggtgctgatggcggccgacatcctgctgtatcagaccaacctggttccggtcggcgaagatgcaaaacaagcactggaactgagccgtgacatcgcccagcgcttcaatgcactgtacggtgaaatttttaaagtcccggaaccgttcatcccgaaaagcggcgcccgtgtgatgtctctgctggaaccgaccaagaaaatgagcaaatctgatgacaaccgcaacaatgttattggcctgctggaagatccgaaaagcgtggtgaagaaaattaaacgtgcggtcaccgattctgacgaaccgccggtcgtgcgctatgatgttcagaacaaagctggtgtctcaaatctgctggatattctgagtgcagtgacgggccagtccatcccggaactggaaaaacaattcgaaggcaaaatgtacggtcatctgaaaggcgaagtcgcagatgctgtgtccggtatgctgaccgaactgcaggaacgttatcaccgttttcgcaatgatgaagccttcctgcagcaagttatgaaagacggcgcagaaaaagcgagtgcccacgcatcccgcacgctgaaagcggtctacgaagcaatcggctttgtggcgaaaccg

SEQ ID NO: 193 - DNA, 1002 bp, synthetic construct
  misc_feature 1..1002: note = artificially synthesized sequence
  source 1..1002: mol_type = other DNA
atgacgaaaccgattgtcttctctggtgctcaaccgtctggtgaactgacgattggtaactacatgggtgctctgcgccagtgggtgaacatgcaggatgactatcattgcatttactgtatcgtggatcaacacgccattaccgttcgtcaggacgctcaaaaactgcgcaaagcgaccctggatacgctggccctgtatctggcatgcggtattgacccggaaaaatcaacgatctttgtgcagtcgcatgttccggaacacgctcaactgggttgggcgctgaactgttatacctactttggcgaactgtcacgtatgacgcagttcaaagataaatcggctcgctatgcggaaaacattaatgcgggtctgtttgattacccggtgctggcggcggccgacatcctgctgtatcagaccaacctggttccggtcggcgaagatcagaaacaacatctggaactgagccgtgacatcgcccagcgcttcaatgcactgtacggtgaaatttttaaagtcccggaaccgttcatcccgaaaagcggcgcccgtgtgatgtctctgctggaaccgaccaagaaaatgagcaaatctgatgacaaccgcaacaatgttattggcctgctggaagatccgaaaagcgtggtgaagaaaattaaacgtgcggtcaccgattctgacgaaccgccggtcgtgcgctatgatgttcagaacaaagctggtgtctcaaatctgctggatattctgagtgcagtgacgggccagtccatcccggaactggaaaaacaattcgaaggcaaaatgtacggtcatctgaaaggcgaagtcgcagatgctgtgtccggtatgctgaccgaactgcaggaacgttatcaccgttttcgcaatgatgaagccttcctgcagcaagttatgaaagacggcgcagaaaaagcgagtgcccacgcatcccgcacgctgaaagcggtctacgaagcaatcggctttgtggcgaaaccg

SEQ ID NO: 194 - DNA, 1002 bp, synthetic construct
  misc_feature 1..1002: note = artificially synthesized sequence
  source 1..1002: mol_type = other DNA
atgacgaaaccgattgtcttctctggtgctcaaccgtctggtgaactgacgattggtaactacatgggtgctctgcgccagtgggtgaacatgcaggatgactatcattgcatttactgtatcgtggatcaacacgccattaccgttcgtcaggacgctcaaaaactgcgcaaagcgaccctggatacgctggccctgtatctggcatgcggtattgacccggaaaaatcaacgatctttgtgcagtcgcatgttccggaacacgctcaactgggttgggcgctgaactgttatacctactttggcgaactgtcacgtatgacgcagttcaaagataaatcggctcgctatgcggaaaacattaatgcgggtctgtttgattacccggtgctggtggcggccgacatcctgctgtatcagaccaacctggttccggtcggcgaagatgcaaaacaagccctggaactgagccgtgacatcgcccagcgcttcaatgcactgtacggtgaaatttttaaagtcccggaaccgttcatcccgaaaagcggcgcccgtgtgatgtctctgctggaaccgaccaagaaaatgagcaaatctgatgacaaccgcaacaatgttattggcctgctggaagatccgaaaagcgtggtgaagaaaattaaacgtgcggtcaccgattctgacgaaccgccggtcgtgcgctatgatgttcagaacaaagctggtgtctcaaatctgctggatattctgagtgcagtgacgggccagtccatcccggaactggaaaaacaattcgaaggcaaaatgtacggtcatctgaaaggcgaagtcgcagatgctgtgtccggtatgctgaccgaactgcaggaacgttatcaccgttttcgcaatgatgaagccttcctgcagcaagttatgaaagacggcgcagaaaaagcgagtgcccacgcatcccgcacgctgaaagcggtctacgaagcaatcggctttgtggcgaaaccg

SEQ ID NO: 195 - DNA, 2580 bp, synthetic construct
  misc_feature 1..2580: note = artificially synthesized sequence
  source 1..2580: mol_type = other DNA
atgcaagaacaataccgcccggaagaaatcgaaagtaaagtccaactgcactgggatgaaaaacgcacctttgaagtcacggaagacgaatctaaagaaaaatattactgcctgagcatgctgccgggtccgtctggtcgtctgcatatgggtcacgtgcgcaactataccattggcgatgttatcgcgcgttaccagcgcatgctgggtaaaaatgttctgcaaccgattggctgggacgcatttggtctgccggctgaaggcgcggccgtgaaaaacaataccgcaccggctccgtggacgtatgataacatcgcatacatgaaaaatcagctgaaaatgctgggcttcggttatgactggtctcgtgaactggcgacctgtacgccggaatattaccgctgggaacaaaaatttttcaccgaactgtacaaaaaaggtctggtctacaagaaaaccagcgcggtgaactggtgcccgaatgatcaaacggtcctggccaacgaacaagtgattgatggctgctgttggcgttgtgacaccaaagttgaacgcaaagaaatcccgcagtggtttattaaaatcacggcgtatgccgatgaactgctgaacgatctggacaaactggatcattggccggacaccgtgaaaacgatgcagcgtaattggattggccgcagcgaaggtgttgaaatcacctttaacgtcaatgattatgacaacaccctgacggtctacaccacgcgtccggataccttcatgggttgcacgtacctggcagtggcagctggtcacccgctggctcagaaagcagcagaaaacaatccggaactggcagcttttattgatgaatgtcgcaataccaaagtggcagaagctgaaatggcaaccatggagaaaaaaggcgtggatacgggttttaaagctgtccatccgctgaccggtgaagaaatcccggtgtgggcggccaacttcgttctgatggaatatggtacgggtgcagtcatggctgtgccgggtcacgatcagcgtgactatgaatttgcctccaaatacggtctgaatattaaaccggttatcctggcagctgatggctcagaaccggacctgtcgcagcaagcactgaccgaaaaaggtgtgctgtttaactcaggcgaattcaatggtctggatcatgaagcggccttcaacgcgattgccgacaaactgaccgcgatgggcgttggtgaacgcaaagtcaattatcgtctgcgcgattggggtgtgtcgcgtcagcgttactggggtgcaccgattccgatggtgaccctggaagatggcaccgttatgccgacgccggatgaccaactgccggttatcctgccggaagatgtggttatggacggtattaccagcccgatcaaagctgatccggaatgggccaaaaccacggtgaatggcatgccggcgctgcgtgaaaccgatacgtttgacaccttcatggaaagctcttggtattacgcccgctatacgtgcccgcagtacaaagaaggcatgctggatagcgaagcagctaactattggctgccggttgacatttacatcggcggtattgaacatgcgatcatgcacctgctgtattttcgtttctttcataaactgatgcgcgatgccggtatggtgaattcagacgaaccggctaaacagctgctgtgtcaaggcatggtcctggcagatgctttctattacgtgggcgaaaacggtgaacgtaattgggtttcgccggtcgatgcgattgttgaacgtgacgaaaaaggtcgcatcgtcaaagccaaagatgcggccggtcacgaactggtgtataccggcatgagcaaaatgagtaaatccaaaaacaacggtatcgatccgcaggttatggtcgaacgttacggcgcggacaccgttcgcctgtttatgatgttcgcgagtccggccgatatgacgctggaatggcaagaatccggcgtggaaggtgccaaccgttttctgaaacgcgtgtggaaactggtttatgaacataccgcaaaaggcgatgtggcagctctgaacgttgacgcactgacggaaaatcagaaagctctgcgtcgcgatgtccacaaaaccattgcaaaagtgacggatgacatcggtcgtcgccaaacctttaacacggcgattgcggccatcatggaactgatgaataaactggcgaaagcaccgaccgatggtgaacaggaccgtgcactgatgcaagaagcactgctggctgtcgtgcgcatgctgaacccgtttaccccgcatatttgcttcacgctgtggcaggaactgaaaggcgaaggtgatatcgacaatgccccgtggccggttgcggatgaaaaagccatggtcgaagacagtaccctggttgtcgtgcaggtcaatggtaaagtgcgcgcgaaaattaccgtgccggttgatgccacggaagaacaagtgcgtgaacgcgcgggccaagaacacctggttgccaaatatctggatggcgtcacggtccgcaaagtcatctatgttccgggtaaactgctgaatctggtcgttggt

SEQ ID NO: 196 - DNA, 92 bp, synthetic construct
  misc_feature 1..92: note = artificially synthesized sequence
  source 1..92: mol_type = other DNA
ggcgtaatacgactcactatagggttaactttaacaaggagaaaaacatgcgttggcgtgactacaaggacgacgacgacaagtaagcttcg

SEQ ID NO: 197 - RNA, 71 nt, synthetic construct
  misc_feature 1..71: note = artificially synthesized sequence
  source 1..71: mol_type = other RNA
gggttaactttaacaaggagaaaaacatgcgttggcgtgactacaaggacgacgacgacaagtaagcttcg

SEQ ID NO: 198 - protein, 12 aa, synthetic construct
  REGION 1..12: note = artificially synthesized sequence
MRWRDYKDDDDK

SEQ ID NO: 199 - protein, 12 aa, synthetic construct
  REGION 1..12: note = artificially synthesized sequence
  SITE 3: note = misc_feature - Xaa is methyl tryptophan
MRXRDYKDDDDK

SEQ ID NO: 200 - DNA, 92 bp, synthetic construct
  misc_feature 1..92: note = artificially synthesized sequence
  source 1..92: mol_type = other DNA
ggcgtaatacgactcactatagggttaactttaacaaggagaaaaacatgcgtctccgtgactacaaggacgacgacgacaagtaagcttcg

SEQ ID NO: 201 - RNA, 71 nt, synthetic construct
  misc_feature 1..71: note = artificially synthesized sequence
  source 1..71: mol_type = other RNA
gggttaactttaacaaggagaaaaacatgcgtctccgtgactacaaggacgacgacgacaagtaagcttcg

SEQ ID NO: 202 - protein, 12 aa, synthetic construct
  REGION 1..12: note = artificially synthesized sequence
MRLRDYKDDDDK

SEQ ID NO: 203 - protein, 12 aa, synthetic construct
  REGION 1..12: note = artificially synthesized sequence
  SITE 3: note = misc_feature - Xaa is methyl leucine
MRXRDYKDDDDK

SEQ ID NO: 204 - DNA, 97 bp, synthetic construct
  misc_feature 1..97: note = artificially synthesized sequence
  source 1..97: mol_type = other DNA
ggcgtaatacgactcactatagggtgattagctcagctgggagagcacctcccttacaaggagggggtcggcggttcgatcccgtcatcacccacca

SEQ ID NO: 205 - RNA, 76 nt, synthetic construct
  misc_feature 1..76: note = artificially synthesized sequence
  source 1..76: mol_type = other RNA
gggtgattagctcagctgggagagcacctcccttacaaggagggggtcggcggttcgatcccgtcatcacccacca

(19) United States
(12) Patent Application Publication
(10) Pub. No.: US 2025/0051784 A1
(43) Pub. Date: Feb. 13, 2025

(54) MODIFIED AMINOACYL-tRNA SYNTHETASE AND USE THEREOF

(71) Applicant: CHUGAI SEIYAKU KABUSHIKI KAISHA, Tokyo (JP)

(72) Inventors: Atsushi OHTA, Kanagawa (JP); Yusuke YAMAGISHI, Kanagawa (JP); Atsushi MATSUO, Shizuoka (JP)

(21) Appl. No.: 18/926,665

(22) Filed: Oct. 25, 2024

Related U.S. Application Data
(62) Division of application No. 17/024,944, filed on Sep. 18, 2020, now Pat. No. 12,163,134.
Application No. 17/024,944 is a division of application No. 15/557,532, filed on Sep. 12, 2017, now Pat. No. 10,815,489.

(30) Foreign Application Priority Data
Mar. 13, 2015 (JP) 2015-051202

(51) Int. Cl. C12N 15/70; C12N 5/10; C12N 9/00; C12N 9/22; C12N 15/09; C12P 19/34; C12P 21/00; C12P 21/02

(52) U.S. Cl. CPC C12N 15/70; C12N 5/10; C12N 9/00; C12N 9/22; C12N 9/93; C12N 15/09; C12P 19/34; C12P 21/00; C12P 21/02

(57) ABSTRACT

The present invention provides modified arninoacyl-tRNA synthetases (ARSs) having increased reactivity with N-methyl amino acids compared to natural aminoacyl-tRNA synthetases. The modified aminoacyI-tRNA synthetases according to the present invention can aminoacylate tRNAs with their corresponding N-methyl-substituted amino acids such as N-methyl-phenylalanine, N-methyl-valine, N-methyl-serine, N-methyl-threonine, N-methyl-tryptophan, and N-methyl-leucine more efficiently than natural aminoacyl-tRNA synthetases. The present invention enables a more efficient production of polypeptides containing N-methyl amino acids.

### CROSS-REFERENCE TO RELATED APPLICATIONS

This application is a divisional of U.S. patent application Ser. No. 17/024,944, filed Sep. 18, 2020, which is a division of U.S. patent application Ser. No. 15/557,532, § 371 date Sep. 12, 2017, now U.S. Pat. No. 10,815,489 B2, issued Oct. 27, 2020, which is a U.S. National Stage of PCT/JP2016/057707, filed Mar. 11, 2016, which claims priority from Japanese application JP 2015-051202, filed Mar. 13, 2015, each of which is incorporated herein by reference in its entirety.

### REFERENCE TO SEQUENCE LISTING SUBMITTED ELECTRONICALLY

The content of the electronically submitted sequence listing (Name: 6663_0335 Sequence_Listing.xml; Size: 310 kilobytes; and Date of Creation: Oct. 24, 2024) filed with the application is incorporated herein by reference in its entirety.

### TECHNICAL FIELD

The present invention relates to aminoacyl-tRNA synthetases (ARSs) that aminoacylate tRNA with the corresponding N-methyl amino acid more efficiently than natural aminoacyl-tRNA synthetases, and use thereof. More specifically, the present invention relates to aminoacyl-tRNA synthetases that have modified amino acid sequences, which are able to aminoacylate a tRNA with one of six N-methyl-substituted amino acids corresponding to the tRNA, namely, N-methyl-phenylalanine, N-methyl-valine, N-methyl-serine, N-methyl-threonine, N-methyl-tryptophan, and N-methyl-leucine, more efficiently than natural ARSs, and uses thereof. The aminoacyl-tRNA synthetases with modified amino acid sequences according to the present invention can be used to produce peptides that selectively and regioselectively contain N-methyl amino acids with high efficiency.

### BACKGROUND ART

Generally in organisms on earth, information stored in DNA (=an information-storing substance) defines, via RNA (=an information-transmitting substance), the structures of proteins (=functional substances) and functions resulting from the structures. Polypeptides and proteins are composed of 20 types of amino acids. The information stored in DNA, which is composed of four kinds of nucleotides, is transcribed into RNA and then translated into amino acids, which make polypeptides and proteins.

During translation, tRNA plays the role of an adaptor that matches a stretch of three nucleotides to one amino acid, and aminoacyl-tRNA synthetase (aminoacyl-tRNA synthetase; ARS) is involved in the attachment of tRNA to its amino acid.

ARSs are enzymes that specifically attach an amino acid to its corresponding tRNA. There are 20 types of ARSs, each corresponding to each of the 20 types of naturally-occurring amino acids with a few exceptions, in every biological species. Of the 20 kinds of proteinaceous amino acids, ARS precisely acylates tRNA having an anticodon corresponding to an arbitrary codon, using the specific amino acid assigned to the codon. In other words, a tRNA synthetase corresponding to a certain amino acid can distinguish a tRNA corresponding to that amino acid from tRNAs corresponding to other amino acids, and does not attach other amino acids.

In translation of an mRNA into a polypeptide chain, a tRNA bound to its corresponding amino acid (aminoacyl-tRNA) pairs with the appropriate codon on the mRNA starting from the initiation codon and form hydrogen bonds with the mRNA on ribosomes. This is followed by peptidyl transfer to the amino acid on the adjacent aminoacyl-tRNA bound corresponding to the next codon. The first tRNA that released the amino acid by the transfer is liberated from the mRNA and can reattach its corresponding amino acid catalyzed by ARS. Translation is terminated upon reaching the mRNA's stop codon and upon arrival of a protein called the termination factor, which releases the polypeptide chain from the ribosome. Proteins within the living body are produced via such processes. The proteins then exert important physiological functions in the living body.

Meanwhile, in the field of pharmaceuticals of which bioactivities are exerted similarly in living bodies, the possibility of creating new pharmaceuticals using compounds having a molecular weight from 500 to 2000, called “middle molecules”, is being anticipated in the field of drug discovery where it was considered difficult to create drugs with conventional low molecular weight compounds having a molecular weight less than 500. A representative example is cyclosporine A, a naturally-derived middle molecule drug, which is a peptide consisting of 11 residues that is produced by microorganisms, inhibits the intracellular target cyclophilin, and can be orally administered.

The Cyclosporine A peptide is characterized by including the non-natural amino acids “N-methyl amino acids” as components. Triggered by this, multiple studies have recently reported that the incorporation of N-methyl amino acids into peptides can increase drug-likeness of the peptides, and this is being further applied to drug discovery (Non Patent Literature 1, 2, 3). Particularly, it has come to be known that the incorporation of N-methyl amino acids leads to decrease in hydrogen bond-donating hydrogens, acquisition of protease resistance, and fixed conformation, and contributes to membrane permeability and metabolic stability (Non Patent Literature 1, 4, Patent Literature 1).

This paves way to the conception of drug discovery methods that select pharmaceutical candidate substances from a library of diverse peptides containing multiple N-methyl amino acids. In terms of diversity and ease of screening, much anticipated are mRNA display libraries of N-methyl amino acid-containing peptides, and such, which use a cell-free translation system (Non Patent Literature 9, 10, Patent Literature 1). First, a display library is constructed, which is a collection of molecules forming one-for-one pairs between an enormous variety of RNA or DNA molecules (genotype) or the like and peptides encoded by these molecules (phenotype). The display library is then allowed to bind to a target protein or the like, followed by washing to remove non-bound molecules and selecting a molecule in the library, which is contained scarcely, in extremely small amounts. The selected RNA (DNA) molecule can be then sequenced to easily obtain the sequence information of the bound peptide. Particularly, an mRNA display library and a ribosome display library utilizing a cell-free translation system can be used to easily analyze 10¹²⁻¹⁴ diverse types of molecules (Non Patent Literature 11). Recently, a method that can prepare peptides containing non-proteinaceous amino acids using a reconstituted cell-free translation system has been also developed, enabling combination with display techniques to construct an N-methyl amino acid-containing peptide display library (Non Patent Literature 10).

Some methods for preparing N-methyl amino acid-containing peptides by translation of mRNAs are known so far. These methods are performed by separately preparing beforehand “N-methyl aminoacyl-tRNAs” and adding these to a translation system.

First, in the pdCpA method developed by Hecht et al. (Non Patent Literature 5), an N-methyl aminoacyl-tRNA is prepared beforehand by ligating pdCpA (5′-phospho-2′-deooxyribocytidylriboadenosine) acylated with a chemically-synthesized non-natural N-methyl amino acid to a tRNA lacking 3′-terminal CA obtained by transcription using T4 RNA ligase. This method has been used to introduce amino acids including N-methylalanine and N-methylphenylalanine (Non Patent Literature 6). However, when the present inventors attempted to introduce multiple N-methyl amino acids by preparing several complexes between non-natural N-methyl amino acids and tRNAs using the pdCpA method and adding the complexes to a cell-free translation system, translational efficiency decreased. Particularly, N-methylvaline could not be introduced (unpublished data).

As a different method, Suga et al. reported a method for aminoacylating tRNAs with N-methyl amino acids activated in advance by esterification using an artificial RNA catalyst (Flexizyme) (Patent Literature 2), and successfully introduced multiple types of N-methyl amino acids by translation. This method can be applied to various side chain structures, but aminoacylation efficiency of amino acids with nonaromatic side chains is in many cases 40 to 60% which is by no means high, and particularly, N-methylvaline introduction has not been confirmed as with the pdCpA method (Non Patent Literature 3).

Moreover, in the method of Szostak et al., aminoacyl-tRNAs are obtained by using tRNAs extracted from Escherichia coli and wild-type ARSs, and then preparing N-methyl aminoacyl-tRNAs via a chemical N-methylation reaction consisting of three steps. However, side chains that are efficiently translated in this method are limited to only the three side chains valine, leucine, and threonine. This method also requires cumbersome operations and further results in contamination of trace amounts of natural amino acids (the starting material) resulting from incomplete progression of the N-methylation reaction. Such difficulties in controlling the reaction directly affect the purity of products (Non Patent Literature 2).

Moreover, since these techniques add N-methyl aminoacyl-tRNAs prepared outside a translation system to a translation reaction solution, N-methyl aminoacyl-tRNAs are not re-produced in the translation system and thus are only consumed in the translation process. This requires addition of large amounts of N-methyl aminoacyl-tRNAs, but this addition itself of large amounts of tRNAs contributes to the reduction of peptide yield (Non Patent Literature 7). Further, instability of aminoacyl-tRNAs in the translation solution becomes problematic. Aminoacyl-tRNAs have been shown to be hydrolyzed under physiological conditions at pH 7.5 due to the presence of ester bonds between amino acids and tRNAs (Non Patent Literature 12). Half-life of aminoacyl-tRNAs depends on amino acid side chains, the shortest being 30 minutes. Hydrolysis of aminoacyl-tRNA is suppressed when it forms a complex with an aminoacyl-tRNA-elongation factor Tu (EF-Tu), but aminoacyl-tRNAs in excess of the concentration of EF-Tu present in the translation system are hydrolyzed. That is to say, as the translation reaction proceeds, deacylation of aminoacyl-tRNAs added at the start of translation proceeds, and at the end, aminoacyl-tRNAs having N-methyl amino acids are exhausted. In fact, Szostak et al. perceived this depletion as a problem and added N-methyl aminoacyl-tRNAs twice, at the start of translation and during the reaction, when synthesizing a polypeptide containing multiple N-methyl amino acids (Non Patent Literature 2).

The above-mentioned problems regarding introduction of N-methyl amino acids can be solved if there are ARSs for N-methyl amino acids having functions similar to natural ARSs for natural amino acids, but ARSs have an ability to precisely recognize their substrates and thus have limitations. As an exception, Murakami et al. reported that natural HisRS and PheRS could be used to introduce N-methylhistidine and N-methylphenylalanine into peptides in a cell-free translation system (Non Patent Literature 8, 13). Also, Szostak et al. confirmed that natural ARSs were used to aminoacylate tRNAs with the six N-methyl amino acids N-methylvaline, N-methylleucine, N-methyl aspartic acid, N-methylhistidine, N-methyllysine, and N-methyltryptophan (Non Patent Literature 14). However, even though a subsequent report using a cell-free translation system and natural ARSs that analyzed translational synthesis of peptides containing N-methylhistidine and N-methyl aspartic acid by mass spectrum reported a certain level of yield, in the case of peptides containing N-methylvaline, N-methylleucine, N-methyllysine, and N-methyltryptophan, it was shown that the efficiency of translational synthesis (ribosomal synthesis) is very low (Non Patent Literature 8). These reports reveal that aminoacylation with N-methyl amino acids and translational introduction of N-methyl amino acids into peptides using natural ARSs has been confirmed substantially in only the three cases of N-methylphenylalanine, N-methylhistidine, and N-methyl aspartic acid.

There are some prior art in which natural ARSs have been modified to give them the function to catalyze the attachment of non-natural amino acids to tRNAs (Patent Literature 3, 4, 5). Even though these are modified ARSs that catalyze the attachment of non-natural amino acids to tRNAs, and substrates of these modified ARSs are amino acids mainly having side chain derivatives of phenylalanine and tyrosine, modified ARSs having N-methyl amino acids as substrates, and modified ARSs that can introduce multiple N-methyl amino acid residues into peptides have not been known.

### CITATION LIST

### Patent Literature

Patent Literature 1: WO2013/100132
Patent Literature 2: WO2007/066627
Patent Literature 3: WO2003/014354
Patent Literature 4: WO2007/103307
Patent Literature 5: WO2002/085923

### Non Patent Literature

Non Patent Literature 1: R. S. Lokey et al., Nat. Chem. Biol. 2011, 7(11), 810-817.
Non Patent Literature 2: J. W. Szostak et al., J. Am. Chem. Soc. 2008, 130, 6131-6136.
Non Patent Literature 3: T. Kawakami et al., Chemistry & Biology, 2008, Vol. 15, 32-42.
Non Patent Literature 4: H. Kessler et al., J. Am. Chem. Soc. 2012, 134, 12125-12133.
Non Patent Literature 5: S. M. Hecht et al., J. Biol. Chem. 253 (1978) 4517-4520.
Non Patent Literature 6: Z. Tan et al., J. Am. Chem. Soc. 2004, 126, 12752-12753.
Non Patent Literature 7: A. O. Subtelny et al., Angew Chem Int Ed 2011 50 3164.
Non Patent Literature 8: M. C. T. Hartman et al., PLoS one, 2007, 10, e972.
Non Patent Literature 9: S. W. Millward et al., J. Am. Chem. Soc., 2005, 127, 14142-14143.
Non Patent Literature 10: Y. Yamagishi et al., Chem. Biol., 18, 1562-1570, 2011.
Non Patent Literature 11: H. R. Hoogenboom, Nature Biotechnol. 23, 1105-1116, 2005.
Non Patent Literature 12: J. R. Peacock et al., RNA, 20, 758-64, 2014.
Non Patent Literature 13: T. Kawakami, ACS Chem Biol., 8, 1205-1214, 2013.
Non Patent Literature 14: M. C. T. Hartman et al., Proc Natl Acad Sci USA., 103, 4356-4361, 2006.

### SUMMARY OF THE INVENTION

### Problems to be Solved by the Invention

An objective of the present invention is to provide modified ARSs that have been modified to increase reactivity, and which use N-methyl amino acids as substrates. More specifically, an objective of the present invention is to provide a novel modified ARS that catalyzes the acylation reaction in which tRNAs attach non-natural N-methyl amino acids, particularly N-methyl-phenylalanine, N-methyl-valine, N-methyl-serine, N-methyl-threonine, N-methyltryptophan, and N-methylleucine, without using large amounts of tRNAs in order to efficiently produce peptides containing multiple N-methyl amino acids, and uses thereof.

### Means for Solving the Problems

In order to obtain ARSs having increased reactivity with N-methyl amino acids, the present inventors obtained multiple ARS genes that employ different amino acids as substrates and introduced mutations into the genes to construct mutated ARS genes encoding ARSs with altered amino acid sequences. These modified ARSs were expressed and collected, and were incubated with tRNAs in the presence of unmodified amino acids or N-methyl amino acids to determine aminoacylation reaction. As a result of estimating the conformation formed in the N-methyl amino acid-ARS interaction, and after much trial and error, the present inventors successfully obtained modified ARSs, including multiple ARSs such as phenylalanyl-tRNA synthetase (PheRS), seryl-tRNA synthetase (SerRS), valyl-tRNA synthetase (ValRS), threonyl-tRNA synthetase (ThrRS), leucyl-tRNA synthetase (LeuRS), and tryptophanyl-tRNA synthetase (TrpRS), with increased activity in the aminoacylation reaction with N-methyl amino acids, compared to wild-type ARSs.

For example, 0.1 μM wild-type PheRS hardly incorporated N-methylphenylalanine into ribosomally-synthesized peptides even when 1 mM N-methylphenylalanine was added. On the other hand, 0.1 μM modified PheRS clearly incorporated N-methylphenylalanine into peptides even when 0.25 mM N-methylphenylalanine was added (Example 1). The amount of phenylalanine and N-methylphenylalanine translationally introduced (ribosomally introduced) into peptides were measured by mass spectroscopy using MALDI-TOF MS. The ratio of the peak values (the peak intensity of the peptide containing N-methylphenylalanine/the peak intensity of the peptide containing phenylalanine) at the time of 0.25 mM N-methylphenylalanine addition was 0.8 when using wild-type PheRS, whereas it dramatically increased to 12.4 when using the modified PheRS α subunit (Example 1). When sequences containing two consecutive and three consecutive phenylalanines were allowed to translate, peptides containing two consecutive and three consecutive N-methylphenylalanines were confirmed to be synthesized respectively, and the efficiency was significantly higher than that using the pdCpA method. Also, ValRS was used to perform translational synthesis in the presence of 5 mM N-methylvaline, and the peptide products were analyzed with mass spectroscopy. A peptide incorporated with unmodified valine was detected as the main product when using wild-type ValRS, whereas a peptide incorporated with N-methylvaline was observed as the main product when using modified ValRS (Example 2). Furthermore, the selectivity to N-methylvaline was successfully increased by introducing mutations into the editing domain of ValRS and decreasing aminoacylation activity with unmodified Val (Example 7). When sequences containing two consecutive and three consecutive valines were allowed to translate, peptides containing two consecutive and three consecutive N-methylvalines were confirmed to be synthesized respectively (Example 2). For SerRS, an N-methylserine-incorporated translation product was detected as the main product when modified SerRS was used under conditions in which an unmodified serine-incorporated translation product is detected as the main product when using wild-type SerRS (Example 3). Even for ThrRS, an N-methyl Thr-incorporated translation product was observed when modified ThrRS was used under conditions in which an unmodified Thr-incorporated translation product is detected when using wild-type ThrRS; whereas it was demonstrated that translation products incorporated with unmodified Thr were hardly observed and the peptide with N-methyl Thr introduced was synthesized with higher purity compared to that achieved with wild-type ThrRS (Example 4). For TrpRS, unmodified Trp was detected as a main product in using wild-type TrpRS, whereas the translation product with N-methyl Trp incorporated was observed as a main product using the modified TrpRS (Example 5). For LeuRS, translation products containing N-methyl Leu were not observed in using wild-type LeuRS, whereas it is revealed that translation products with N-methyl Leu incorporated were produced as much as translation products containing unmodified Leu when modified LeuRSs were used (Example 6). Thus, the modified ARSs according to the present invention can be used to introduce N-methyl amino acids into peptides more efficiently than wild-type ARS.

Accordingly, the following inventions are provided.

The present invention provides ARSs having reactivity with N-methyl amino acids. Specifically, the present invention relates to an aminoacyl-tRNA synthetase (aminoacyl-tRNA synthetase; ARS) that has an altered amino acid sequence and is able to incorporate any N-methyl amino acid, particularly the six N-methyl-substituted amino acids of N-methyl-phenylalanine, N-methyl-valine, N-methyl-serine, N-methyl-threonine, N-methyltryptophan, and N-methylleucine more efficiently than natural ARSs and use thereof. The ARS with an altered amino acid sequence according to the present invention can be used to produce peptides selectively and regioselectively containing any N-methyl amino acid from among these N-methyl amino acids with high efficiency.

The present invention further relates to a method for producing polypeptides containing non-natural amino acids using a modified ARS according to the present invention. More specifically, the present invention relates to a method for producing polypeptides containing N-methylphenylalanine, N-methylvaline, N-methylserine, N-methylthreonine, N-methyltryptophan, and N-methylleucine using ARSs for phenylalanine, valine, serine, threonine, tryptophan, and leucine, respectively, with altered amino acid sequences.

Namely, the present invention provides the following inventions:

[1]A polypeptide comprising a modified aminoacyl-tRNA synthetase (ARS), wherein the ARS is able to incorporate an N-methyl amino acid more efficiently than an original, natural ARS.
[2]A polypeptide modified to enhance an aminoacylation reaction with an N-methyl amino acid, wherein the polypeptide has aminoacyl-tRNA synthetase (ARS) activity, wherein the modification comprises at least one amino acid substitution that causes a decrease of 10 or more in molecular weight.
[3] The polypeptide according to [1] or [2], wherein the N-methyl amino acid is selected from the group consisting of valine, serine, phenylalanine, threonine, tryptophan, and leucine.
[4] The polypeptide according to any one of [1] to [3], wherein the ARS is selected from the group consisting of ValRS, SerRS, PheRS a subunit, ThrRS, TrpRS, and LeuRS.
[5] The polypeptide according to any one of [1] to [4], wherein the ValRS is modified at a position(s) corresponding to asparagine at position 43 and/or threonine at position 45 and/or threonine at position 279 of ValRS from Escherichia coli.
[6] The polypeptide according to any one of [1] to [4], wherein the SerRS is modified at a position(s) corresponding to glutamic acid at position 239 and/or threonine at position 237 of SerRS from E. coli.
[7] The polypeptide according to any one of [1] to [4], wherein the PheRS a subunit is modified at a position corresponding to glutamine at position 169 of PheRS a subunit from E. coli.
[8] The polypeptide according to any one of [1] to [4], wherein the ThrRS is modified at a position(s) corresponding to methionine at position 332 and/or histidine at position 511 of ThrRS from E. coli.
[9] The polypeptide according to any one of [1] to [4], wherein the TrpRS is modified at a position(s) corresponding to methionine at position 132 and/or glutamine at position 150 and/or histidine at position 153 of TrpRS from E. coli.
[10] The polypeptide according to any one of [1] to [4], wherein the LeuRS is modified at a position corresponding to tyrosine at position 43 of LeuRS from E. coli.
[11] The polypeptide according to any one of [1] to [4], wherein the ValRS has (a) glycine or alanine at a position corresponding to asparagine at position 43 and/or (b) serine at a position corresponding to threonine at position 45 and/or (c) glycine or alanine at a position corresponding to threonine at position 279 of ValRS from E. coli.
[12] The polypeptide according to any one of [1] to [4], wherein the SerRS has (a) glycine or alanine at a position corresponding to glutamic acid at position 239 and/or (b) serine at a position corresponding to threonine at position 237 of SerRS from E. coli.
[13] The polypeptide according to any one of [1] to [4], wherein the PheRS a subunit has glycine or alanine at a position corresponding to glutamine at position 169 of PheRS a subunit from E. coli.
[14] The polypeptide according to any one of [1] to [4], wherein the ThrRS has glycine at a position corresponding to methionine at position 332 and/or histidine at position 511 of ThrRS from E. coli.
[15] The polypeptide according to any one of [1] to [4], wherein the TrpRS has (a) alanine or valine at a position corresponding to methionine at position 132 and/or (b) alanine at a position corresponding to glutamine at position 150 and/or (c) alanine at a position corresponding to histidine at position 153 of TrpRS from E. coli.
[16] The polypeptide according to any one of [1] to [4], wherein the LeuRS has glycine at a position corresponding to tyrosine at position 43 of LeuRS from E. coli.
[17] The polypeptide according to any one of [1] to [16], wherein the ARS is derived from a bacterium.
[18] The polypeptide according to [17], wherein the bacterium is Escherichia coli.
[19] The polypeptide according to any one of [1] to [18], wherein the polypeptide is selected from the group consisting of the following (a) and (b):
(a) a polypeptide comprising amino acids selected from the group consisting of SEQ ID NOs: 1 to 11 and 182 to 187, and
(b) a polypeptide comprising an amino acid sequence having at least 90% identity to an amino acid sequence selected from the group consisting of SEQ ID NOs: 1 to 11 and 182 to 187.
   [20] An isolated polypeptide comprising an amino acid sequence selected from the group consisting of SEQ ID NOs: 1 to 11 and 182 to 187.
   [21]A fusion polypeptide of a polypeptide according to any one of [1] to [20] and another polypeptide.
   [22]A polynucleotide encoding a polypeptide according to any one of [1] to [21].
   [23]A vector comprising the polynucleotide according to [22].
   [24]A host cell comprising the polynucleotide according to [22] or the vector according to [23].
   [25]A method for producing a polypeptide according to any one of [1] to [21], comprising the step of culturing the host cell according to [24].
   [26]A method for producing a tRNA acylated with an N-methyl amino acid, comprising the step of contacting the N-methyl amino acid with a tRNA in the presence of the polypeptide according to any one of [1] to [20].
   [27] The method according to [26], wherein the step of contacting is carried out in a cell-free translation system.
   [28]A method for producing a polypeptide comprising an N-methyl amino acid, comprising the step of performing translation in the presence of the polypeptide according to any one of [1] to [20] and the N-methyl amino acid.
   [29] The method according to [28], wherein the step of performing translation is carried out in a cell-free translation system.

### Effects of the Invention

The modified ARSs according to the present invention can be used to efficiently attach N-methylphenylalanine, N-methylvaline, N-methylthreonine, N-methylserine, N-methyltryptophan, and N-methylleucine to tRNAs corresponding to natural phenylalanine, valine, threonine, serine, tryptophan, and leucine, respectively, without complicated reactions.

Methods using modified ARSs according to the present invention require no stoichiometric amount of tRNA, can synthesize peptides into which multiple N-methyl amino acids are introduced with a high translational efficiency, and is useful for generating a highly diverse peptide library.

In order to investigate the effect on translational efficiency provided by the ARS's characteristic feature of “providing a continuous supply of aminoacyl-tRNA during the translation reaction”, the present inventors compared the introduction efficiencies of N-methylphenylalanine in two methods: a method using the modified PheRS05 (SEQ ID NO: 2) obtained by the present application invention and the pdCpA method in which aminoacyl-tRNA is not expected to be regenerated during the translation reaction. As a result, the translational efficiency of the method using the modified ARS was higher, and particularly, when two consecutive and three consecutive N-methylphenylalanines were introduced, the target peptide was synthesized approximately 4 to 8 times more (unpublished data). Thus, the present invention enables more efficient production of N-methyl amino acid-containing polypeptides, which were conventionally hard to produce.

### BRIEF DESCRIPTION OF DRAWINGS

FIG. 1 shows evaluation of aminoacylation activities of modified ARSs. The bands of the synthetic peptide acylated with N-methylphenylalanine using PheRS04 and PheRS05 were detected more strongly than when using wild-type PheRS (lane 8, 9 vs. 12, 13).

FIG. 2 shows the confirmation by electrophoresis of peptides ribosomally synthesized using mutant PheRS. The bands of the N-methylphenylalanine-containing peptide synthesized using the modified PheRS were detected more strongly than that using wild-type PheRS (lanes 2, 3 vs. 5, 6).

FIG. 3-1 shows detection by mass spectroscopy of peptides ribosomally synthesized using the modified PheRS. Mass spectrum of the peptide ribosomally synthesized using (a) 0.1 μM wt PheRS and 0.25 mM Phe, (b) 0.1 μM wt PheRS and 0.25 mM MePhe, or (c) 0.1 μM wt PheRS and 1 mM MePhe.

FIG. 3-2 shows detection by mass spectroscopy of peptides ribosomally synthesized using the modified PheRS. Mass spectrum of the peptide ribosomally synthesized using (d) 0.1 μM PheRS05 and 0.25 mM Phe, (e) 0.1 μM PheRS05 and 0.25 mM MePhe, or (f) 0.1 μM PheRS05 and 1 mM MePhe.

FIG. 4 shows aminoacylation reaction performed using modified ValRSs. tRNA acylated with N-methylvaline using the mutant 13 (ValRS13) was observed more than tRNA acylated using wild-type ValRS (lane 10).

FIG. 5 shows the results of mass spectrometry analysis of peptides translated using the modified ValRSs. Mass spectrum of the peptides translated using (a) wild-type ValRS, (b) ValRS13, or (c) ValRS04. The peptide containing MeVal was observed as a main product when ValRS13 was used.

FIG. 6 shows aminoacylation reaction performed using ValRS13-11. tRNA acylated with N-methylvaline using the mutant 13-11 (ValRS13-11) was observed more than tRNA acylated using wild-type ValRS or the mutant 13 (ValRS13).

FIG. 7 shows comparison of activities between ValRS13 and ValRS13-11. Mass spectra of the N-methyl peptide-containing peptides translated using ValRS13 ((a), (c), (e)) and ValRS13-11 ((b), (d), (f)). It can be seen that the N-methylvaline-containing target peptide translated using ValRS13-11 had higher purity.

FIG. 8 shows aminoacylation reaction performed using modified SerRSs. tRNA acylated with N-methylserine was observed when mutants 03, 35, and 37 were used (lanes 3, 25, 27).

FIG. 9 shows the results of mass spectrometry analysis of peptides ribosomally synthesized using the modified SerRSs. Mass spectrum of the N-methylserine-containing peptide translated using (a) wild-type SerRS, (b) SerRS03, (c) SerRS05, (d) SerRS35, or (e) SerRS37. The MeSer-containing target peptide synthesized using each modified SerRS had higher purity compared to that synthesized using wild-type SerRS.

FIG. 10 shows the results of mass spectrometry analysis of peptides ribosomally synthesized using modified ThrRSs. Mass spectrum of the N-methylthreonine-containing peptide translated using (a) wild-type ThrRS, (b) ThrRS03, or (c) ThrRS14. The MeThr-containing target peptide synthesized using each modified ThrRS had higher purity compared to that synthesized using wild-type ThrRS.

FIG. 11 shows the results of mass spectrometry analysis of peptides ribosomally synthesized using modified TrpRSs. Mass spectrum of the peptide translated using (a) wild-type TrpRS, (b) TrpRS04, (c) TrpRS05, or (d) TrpRS18. The MeW-containing peptide was observed as a main product when TrpRS04, 05, and 18 were used.

FIG. 12 shows the results of mass spectrometry analysis of peptides ribosomally synthesized using the modified LeuRS. Mass spectrum of the peptide translated using (a) wild-type LeuRS or (b) LeuRS02. The MeL-containing peptide was observed as a main product when LeuRS02 was used.

FIG. 13 shows aminoacylation reaction with MeVal using the modified ValRSs having mutation in the editing domain. The activities of three mutants, 13-11, 66, and 67 were not much different (lanes 17, 18, 19).

FIG. 14 shows aminoacylation reaction with Val using the modified ValRSs having mutation in the editing domain. The activities of the mutants 66 and 67 were attenuated compared to the activity of the mutant 13-11 (lane 17 vs 18, 19).

### MODE FOR CARRYING OUT THE INVENTION

An objective of the present invention is to provide mutant enzymes with altered enzyme-substrate specificity for aminoacyl-tRNA synthetase. More specifically, the present invention is characterized by the preparation of mutant aminoacyl-tRNA synthetases that can efficiently and selectively produce polypeptides containing N-methyl amino acids in large amounts, by altering the amino acid sequence of natural aminoacyl-tRNA synthetase.

The present invention provides polypeptides including ARSs that can react with N-methyl amino acids. More specifically, the present invention provides polypeptides comprising modified ARSs that can react with N-methyl amino acids more efficiently than the original, natural ARSs. The present invention also provides polypeptides comprising modified ARSs that can incorporate N-methyl amino acids more efficiently than the original, natural ARSs. The polypeptides according to the present invention are polypeptides that have aminoacyl-tRNA synthetase activity and are modified to enhance the aminoacylation reaction with N-methyl amino acids. As used herein, the phrase “incorporate N-methyl amino acids” refers to, for example, aminoacylation of tRNAs with N-methyl amino acids corresponding to the tRNAs and may be attachment of the N-methyl amino acids to the tRNAs or incorporation of N-methyl amino acids to proteins synthesized in a translation reaction using the aminoacyl-tRNA produced in the acylation reaction. The phrase “a polypeptide has aminoacyl-tRNA synthetase activity” includes not only the case in which the polypeptide exhibits aminoacyl-tRNA synthetase activity by itself, but also the case in which the polypeptide exhibits aminoacyl-tRNA synthetase activity together with other factors. For example, when an aminoacyl-tRNA synthetase is composed of multiple subunits, the polypeptide according to the present invention may be one subunit or may exhibit aminoacyl-tRNA synthetase activity as a complex with other subunits. In such a case, when an aminoacyl-tRNA synthetase complex is formed between a modified polypeptide according to the present invention and other wild-type subunits, the aminoacyl-tRNA synthetase complex can enhance aminoacylation reaction with N-methyl amino acids more than an aminoacyl-tRNA synthetase complex consisting of wild-type subunits. That is to say, in the present invention, polypeptides with aminoacyl-tRNA synthetase activity modified to enhance aminoacylation reaction with N-methyl amino acids include a polypeptide that is one modified subunit in an aminoacyl-tRNA synthetase complex consisting of multiple subunits and is modified to enhance aminoacylation reaction with N-methyl amino acids by the aminoacyl-tRNA synthetase complex.

A polypeptide comprising a modified ARS refers to a polypeptide comprising the polypeptide chain of the modified ARS, and specifically the polypeptide is a polypeptide comprising the amino acid sequence of the modified ARS. The original, natural ARS refers to a natural ARS from which modified ARS is derived, may be for example a wild-type ARS, and includes a naturally-occurring polymorphism. The phrase “can react with N-methyl amino acids” means that modified ARSs can perform an enzymatic reaction with N-methyl amino acids as substrates. The reaction may be, for example, an acylation reaction of tRNAs with N-methyl amino acids, and specifically a reaction that catalyzes a coupling reaction between an N-methyl amino acid and a tRNA. For example, depending on an amino acid used as a substrate by an ARS, the reaction is performed in the presence of the appropriate N-methyl amino acid and the appropriate tRNA, and the coupling between the N-methyl amino acid and the tRNA may be detected. Alternatively, a reaction with N-methyl amino acids may be incorporation of N-methyl amino acids into polypeptides in translation. Production of N-methyl amino acid-tRNA by ARSs can be detected by, for example, performing translation in the presence of the modified ARSs and N-methyl amino acids and detecting incorporation of N-methyl amino acids into the polypeptides produced by translation. The reactivity with N-methyl amino acids is considered to be higher when N-methyl amino acids are frequently incorporated into polypeptides.

The phrase “a modified ARS can react more efficiently than the original, natural ARS” may mean that the modified ARS reacts more efficiently than the original, natural ARS at least under a certain condition, or that a reaction or reaction product that cannot be observed when using the original ARS can be observed when using the modified ARS. For example, a modified ARS is considered to react with an N-methyl amino acid more efficiently than the original, natural ARS when the modified ARS produces polypeptides containing the N-methyl amino acid more than the original, natural ARS. A modified ARS is considered to react with its corresponding N-methyl amino acid more efficiently than the original, natural ARS when, for example, production of polypeptides containing the N-methyl amino acid that cannot be observed when using the original, natural ARS can be observed when using the modified ARS. For example, a modified ARS is considered to react with an N-methyl amino acid more efficiently than the original, natural ARS when, for example, production of polypeptides containing 2, 3, or more consecutive N-methyl amino acids cannot be observed when using the original, natural ARS, but can be observed when using the modified ARS.

The phrase “can react more efficiently than the original, natural ARS” may mean that a reactant of interest is purified at least under a certain condition with higher purity compared to the purity achieved with the original, natural ARS. For example, a modified ARS is considered to react with its corresponding N-methyl amino acid more efficiently than the original, natural ARS when the modified ARS produces polypeptides containing its corresponding N-methyl amino acid more than the original, natural ARS, relative to natural amino acids derived as a result of contamination, Alternatively, a modified ARS is considered to react with N-methyl amino acids more efficiently than the original ARS when it is confirmed that reaction efficiency of the modified ARS for the N-methyl amino acid remains unchanged and reaction efficiency of the modified ARS for natural amino acids decreases. For example, a modified ARS is considered to react with its corresponding N-methyl amino acid more efficiently than the original, natural ARS when reactivity of the modified ARS to the N-methyl amino acid is relatively higher than reactivity of the modified ARS to natural amino acids.

N-methyl amino acids are not particularly limited, but are appropriately selected based on ARSs. For example, when the modified ARS is valine ARS (ValRS), N-methyl amino acid is N-methylvaline; when the modified ARS is threonine ARS (Thr), N-methyl amino acid is N-methylthreonine; when the modified ARS is serine ARS (SerRS), N-methyl amino acid is N-methylserine; when the modified ARS is phenylalanine ARS a subunit (PheRS), N-methyl amino acid is N-methylphenylalanine; when the modified ARS is tryptophan ARS (TrpRS), N-methyl amino acid is N-methyltryptophan; when the modified ARS is leucine ARS (LeuRS), N-methyl amino acid is N-methylleucine.

For example in ValRS, modified sites of ARS are preferably the position(s) corresponding to asparagine at position 43 and/or threonine at position 45 and/or threonine at position 279 of ValRS from E. coli. Modified sites of ARS are preferably a combination of any two positions selected from the positions corresponding to asparagine at position 43, threonine at position 45, and threonine at position 279 (e.g., a combination of position 43 and position 45, position 43 and position 279, or position 45 and position 279), and more preferably a combination of positions corresponding to asparagine at position 43, threonine at position 45, and threonine at position 279. SerRS can be modified at the position(s) corresponding to glutamic acid at position 239 and/or threonine at position 237 of SerRS from E. coli, and more preferably, can be modified at a combination of the positions corresponding to glutamic acid at position 239 and threonine at position 237. PheRS a subunit is preferably modified at the position corresponding to glutamine at position 169 of PheRS from E. coli. ThrRS can be modified at the position(s) corresponding to methionine at position 332 and/or histidine at position 511 of ThrRS from E. coli. TrpRS can be modified at the position(s) corresponding to methionine at position 132 and/or glutamine at position 150 and/or histidine at position 153 of TrpRS from E. coli, preferably can be modified at a combination of any two positions selected from positions corresponding to methionine at position 132, glutamine at position 150, and histidine at position 153 (e.g., a combination of position 132 and position 150, position 132 and position 153, or position 150 and position 153), and more preferably can be modified at a combination of the positions corresponding to methionine at position 132, glutamine at position 150, and histidine at position 153. LeuRS can be modified at the position corresponding to tyrosine at position 43 of LeuRS from E. coli. It should be noted that these modified ARSs may be further modified at other positions. The position numbers in each ARS are indicated taking the position number of the starting methionine in each ARS from E. coli as 1. Specifically, the position numbers in each ARS are indicated taking the position number of the first methionine in the sequences of P07118 (SEQ ID NO: 24) for ValRS, P08312 (SEQ ID NO: 28) for PheRS α subunit, P0A8M3 (SEQ ID NO: 29) for ThrRS, P0A8L1 (SEQ ID NO: 26) for SerRS, P00954 (SEQ ID NO: 188) for TrpRS, and P07813 (SEQ ID NO: 189) for LeuRS (UniProt (http://www.uniprot.org/) as 1. In a certain ARS, a position corresponding to a certain amino acid in ARS from E. coli refers to the amino acid located in the site corresponding to the amino acid in ARS from E. coli and can be identified based on structural similarity between the certain ARS and ARS from E. coli. For example, the corresponding amino acid can be identified as the amino acid aligned at the position of the amino acid in ARS from E. coli when the amino acid sequences of an ARS of interest and ARS from E. coli are aligned. As used herein, a position corresponding to a certain amino acid in ARS from E. coli is preferably the position sterically corresponding to the certain amino acid in ARS from E. coli. The sterically corresponding position refers to the position of an amino acid corresponding to the position of the certain amino acid in ARS from E. coli in the conformation of ARS.

Those skilled in the art can easily identify the sterically corresponding position by aligning ARS from E. coli with all known ARSs from other biological species for example using Multiple Sequence Alignment with default parameters in ClustalW ver2.1 (http://clustalw.ddbj.nig.ac.jp). Particularly, ARSs of interest are not limited to those from prokaryotes. Generally, sequences of ARSs in eukaryotes comprise various functional domains in addition to catalytic domain, and the sequence identity between ARSs from eukaryotes and ARSs from prokaryotes is not always high. In contrast, sequences of catalytic sites, including amino acid recognition site, and editing domain are highly conserved, and the sterically corresponding position in ARSs from eukaryotes can be easily identified using publicly-available alignment techniques.

For example, the sites corresponding to positions 43 and 45 of E. coli ValRS may be respectively amino acid sites of “N/Y/T” and “T/S” in PPP(N/Y/T)X(T/S)G motif (SEQ ID NO: 180; “N/Y/T” is preferably N; X is any amino acid, preferably V, I, or P, and more preferably V; “T/S” is preferably T) present in ValRS from other organisms. More preferably, the sites corresponding to positions 43 and 45 of E. coli ValRS may be amino acids at N and T respectively in PPPNXTG motif (SEQ ID NO: 181; X is any amino acid, preferably V, I, or P, and more preferably V) present in ValRS from other organisms. For example, the position corresponding to asparagine at position 43 of E. coli ValRS is asparagine at position 345 in human (Uniprot P26640) and asparagine at position 191 in Saccharomyces cervisiae (Uniprot P07806).

Modifications of ARSs include preferably at least one substitution with an amino acid that causes a decrease of 10 or more in molecular weight. Such modifications include, for example, a modification of an amino acid selected from the group consisting of amino acids other than Thr (T), such as Gln (Q), Asn (N), Glu (E), Met (M), Tyr (Y), and His (H), into Ala (A) or Gly (G) (preferably into Gly), for example, a modification of Thr (T) into Ser (S), and for example, a modification of Met (M) into Val (V).

Amino acids to be modified may be appropriately selected. For example in ValRS, the position corresponding to asparagine at position 43 of ValRS from E. coli is preferably modified into glycine or alanine, the position corresponding to threonine at position 45 of ValRS from E. coli is preferably modified into serine, and the position corresponding to threonine at position 279 of ValRS from E. coli is preferably modified into glycine or alanine. In SerRS, the position corresponding to glutamic acid at position 239 of SerRS from E. coli is preferably modified into glycine or alanine, and the position corresponding to threonine at position 237 of SerRS from E. coli is preferably modified into serine. In PheRS, the position corresponding to glutamine at position 169 of PheRS α subunit from E. coli is preferably modified into glycine or alanine. In ThrRS, the position corresponding to methionine at position 332 of ThrRS from E. coli is preferably modified into glycine, and the position corresponding to histidine at position 511 of ThrRS from E. coli is preferably modified into glycine. In TrpRS, the position corresponding to methionine at position 132 of TrpRS from E. coli is preferably modified into valine or alanine, the position corresponding to glutamine at position 150 of TrpRS from E. coli is preferably modified into alanine, and the position corresponding to histidine at position 153 of TrpRS from E. coli is preferably modified into alanine. In LeuRS, the position corresponding to tyrosine at position 43 of LeuRS from E. coli is preferably modified into glycine.

Specifically, the present invention includes the following polypeptides:
(a) a polypeptide comprising the amino acid sequence of any of SEQ ID NOs: 3-5, 182, and 183 (ValRS04, ValRS13, ValRS13-11, ValRS66, and ValRS67); and
(b) a polypeptide that has reactivity with N-methyl Val, has at least 90% identity to the amino acid sequence of any of SEQ ID NOs: 3-5, 182, and 183, and comprises at least one amino acid of the following (i) to (iii):
(i) Gly or Ala at the amino acid position corresponding to position 43 of SEQ ID NOs: 3-5, 182, and 183;
(ii) Ser at the amino acid position corresponding to position 45 of SEQ ID NOs: 3-5, 182, and 183; and
(iii) Gly or Ala at the amino acid position corresponding to position 279 of SEQ ID NOs: 3-5, 182, and 183.

Furthermore, the above-mentioned reactivity is preferably higher than the reactivity of a polypeptide having (i) Asn at the amino acid position corresponding to position 43 and/or (ii) Thr at the amino acid position corresponding to position 45 and/or (iii) Thr at the amino acid position corresponding to position 279. For example, the ValRS according to the present invention preferably has a reactivity with N-methyl Val higher than the reactivity of a ValRS having Asn at the amino acid position corresponding to position 43, Thr at the amino acid position corresponding to position 45, and Thr at the amino acid position corresponding to position 279 of the ValRS.

The present invention also includes the following polypeptides:
(a) a polypeptide comprising the amino acid sequence of any of SEQ ID NOs: 6-9 (SerRS03, SerRS05, SerRS35, and SerRS37) and
(b) a polypeptide that has reactivity with N-methyl Ser, has at least 90% identity to the amino acid sequence of any of SEQ ID NOs: 6-9, and comprises at least one amino acid of the following (i) and (ii);
(i) Ser at the amino acid position corresponding to position 237 of SEQ ID NOs: 6-9, and
(ii) Gly or Ala at the amino acid position corresponding to position 239 of SEQ ID NOs: 6-9.

Furthermore, the above-mentioned reactivity is preferably higher than the reactivity of a polypeptide having (i) Thr at the amino acid position corresponding to position 237 and/or (ii) Glu at the amino acid position corresponding to position 239. For example, the SerRS according to the present invention preferably has a reactivity with N-methyl Ser higher than the reactivity of a SerRS having Thr at the amino acid position corresponding to position 237 of the SerRS and Glu at the amino acid position corresponding to position 239 of the SerRS.

The present invention also includes the following polypeptides:
(a) a polypeptide comprising the amino acid sequence of any of SEQ ID NOs: 1-2 (PheRS05 and PheRS04); and
(b) a polypeptide that has reactivity with N-methyl Phe, has at least 90% identity to the amino acid sequence of any of SEQ ID NOs: 1-2, and comprises an amino acid sequence in which the amino acid at the position corresponding to position 169 of any of SEQ ID NOs: 1-2 is Gly or Ala. Furthermore, the above-mentioned reactivity is preferably higher than the reactivity of a polypeptide in which the amino acid at the above-mentioned position is Gln. The polypeptides as described above represent ARS α subunit, and therefore the polypeptides can form a complex with β subunit to result in a functional ARS. β subunit is not particularly limited, but may be, for example, a desired wild-type subunit. As an example, β subunit that can be used is one comprising the amino acid sequence of NCBI Reference Sequence WP_000672380 (e.g., WP_000672380.1) (wherein the base sequence represents 1897337-1899721 of GenBank CP009685 (e.g., CP009685.1)).

The present invention also includes the following polypeptides:
(a) a polypeptide comprising the amino acid sequence of any of SEQ ID NOs: 10-11 (ThrRS03 and ThrRS14); and
(b) a polypeptide that has reactivity with N-methyl Thr, has at least 90% identity to the amino acid sequence of any of SEQ ID NOs: 10-11, and comprises at least one amino acid of the following (i) and (ii):
(i) Gly at the amino acid position corresponding to position 332 of SEQ ID NOs: 10-11; and
(ii) Gly at the amino acid position corresponding to position 511 of SEQ ID NOs: 10-11.

Furthermore, the above-mentioned reactivity is preferably higher than the reactivity of a polypeptide having (i) Met at the amino acid position corresponding to position 332 and/or (ii) His at the amino acid position corresponding to position 511. For example, the ThrRS according to the present invention preferably has a reactivity with N-methyl Thr higher than the reactivity of a ThrRS having Met at the amino acid position corresponding to position 332 of the ThrRS and His at the amino acid position corresponding to position 511 of the ThrRS.

The present invention also includes the following polypeptides:
(a) a polypeptide comprising the amino acid sequence of any of SEQ ID NOs: 184-186 (TrpRS04, TrpRS05, and TrpRS18); and
(b) a polypeptide that has reactivity with N-methyl Trp, has at least 90% identity to the amino acid sequence of any of SEQ ID NOs: 184-186, and comprises at least one amino acid according to any of the following (i) to (iii):
(i) Val or Ala at the amino acid position corresponding to position 132 of SEQ ID NOs: 184-186;
(ii) Ala at the amino acid position corresponding to position 150 of SEQ ID NOs: 184-186; and
(iii) Ala at the amino acid position corresponding to position 153 of SEQ ID NOs: 184-186.

Furthermore, the above-mentioned reactivity is preferably higher than the reactivity of a polypeptide having (i) Met at the amino acid position corresponding to position 132 and/or (ii) Gln at the amino acid position corresponding to position 150 and/or (iii) His at the amino acid position corresponding to position 153. For example, the TrpRS according to the present invention preferably has a reactivity with N-methyl Trp higher than the reactivity of a TrpRS having Met at the amino acid position corresponding to position 132 of the TrpRS, Gln at the amino acid position corresponding to position 150 of the TrpRS, and His at the amino acid position corresponding to position 153 of the TrpRS.

The present invention also includes the following polypeptides:
(a) a polypeptide comprising the amino acid sequence of SEQ ID NO: 187 (LeuRS02); and
(b) a polypeptide that has reactivity with N-methyl Leu, has at least 90% identity to the amino acid sequence of SEQ ID NO: 187, and comprises an amino acid sequence in which the amino acid at the position corresponding to position 43 of SEQ ID NO: 187 is Gly.

Furthermore, the above-mentioned reactivity is preferably higher than the reactivity of a polypeptide having (i) Tyr at the amino acid position corresponding to position 43.

The identity of amino acid sequence is preferably 93% or more, more preferably 95% or more, more preferably 97% or more, 98% or more, or 99% or more.

The N-methyl aminoacyl-tRNA synthetases according to the present invention are characterized by the ability to efficiently acylate tRNAs with N-methyl amino acids, which are non-natural amino acids known to enhance drug-likeness of peptides. The N-methyl aminoacyl-tRNA synthetases according to the present invention may be derived from any organism including bacteria such as E. coli, yeast, animals, or plants, but, because of their versatility, preferable are those that have high sequence conservation with aminoacyl-tRNA synthetases exemplified in the Examples herein (SEQ ID NOs: 1-11, 182-187), and thus have mutation site(s) easily identified in other biological species. For example, polypeptides comprising the modified ARSs according to the present invention may be derived from ARSs from eukaryotes or prokaryotes. Eukaryotes include protists (including protozoan and unicellular green algae), fungi (including ascomycetes and basidiomycetes), plants (including bryophytes, pteridophytes, and seed plants (gymnosperms and angiosperms)), and animals (including invertebrates and vertebrates). Prokaryotes include archaebacteria (including thermophiles and methane bacteria) and eubacteria (including cyanobacteria and E. coli). Polypeptides comprising the modified ARSs according to the present invention may be derived from mammals (such as human, mouse, rat, guinea pig, rabbit, sheep, monkey, goat, donkey, cattle, horse, and pig). Polypeptides comprising the modified ARSs according to the present invention may be derived from, for example, E. coli or yeast, and preferably derived from prokaryotes, for example bacteria. The polypeptides according to the present invention are derived from, for example, bacteria of the family Enterobacteriaceae, for example, E. coli, including, but are not limited to, for example, the genera Escherichia (including E. coli, E. albertii, E. fergusonii), Shigella (including S. dysenteriae, S. flexneri, S. boydii, S. sonnei, S. enterica, S. bongori), Citrobacter (including C. rodentium, C. koseri, C. farmeri, C. youngae), Kluyvera (including K. ascorbata), Trabulsiella (including T. guamensis), Klebsiella, and the like.

N-methyl aminoacyl-tRNA synthetases from E. coli were used in the Examples herein as one example, and therefore the modified positions indicated herein are positions in E. coli. When N-methyl aminoacyl-tRNA synthetases from other organisms are used, positions to be modified are amino acids at positions corresponding to those in the amino acid sequence of N-methyl aminoacyl-tRNA synthetases from E. coli based on sequence homology. ARSs are generally very highly conserved because they are enzymes playing an essential part in the translation mechanism that exists in all organisms. Accordingly, a desired ARS can be modified based on the method according to the present invention to obtain a modified ARS with an increased ability to incorporate an N-methyl amino acid.

Amino acids to be newly introduced into N-methyl aminoacyl-tRNA synthetases are selected based on hydrophilicity, hydrogen bonds, and side chain size of amino acids in consideration of the distance to and interaction with N-methyl group. For example, when avoiding the steric repulsion between the amino acid at the position and an N-methyl group, the distance to the N-methyl amino group can be adjusted by, for example, substituting a high molecular weight amino acid with a low molecular weight amino acid. Specifically, the distance can be adjusted by reducing the molecular weight, for example, by substituting threonine (Thr) with serine (Ser).

For example, when an amino acid to be modified in an N-methyl aminoacyl-tRNA synthetase is asparagine, the low molecular weight amino acids can include, but are not limited to, for example, serine, valine, glycine, aspartic acid, and alanine. When an amino acid to be modified is glutamic acid, the low molecular weight amino acids can include, but are not limited to, for example, alanine, valine, serine, alanine, and aspartic acid. Moreover, for example, when an amino acid to be modified is Thr, the low molecular weight amino acids include preferably, for example, Ser. When an amino acid to be modified is an amino acid other than the amino acids as described above, the low molecular weight amino acids include preferably, for example, glycine (Gly) and alanine (Ala), and more preferably glycine (Gly). To give one specific example, Thr can be substituted with Ser; Gln, Glu, and Asn can be substituted with Gly or Ala; and Met, His, Gln, and the like can be substituted with Gly, but the substitutions are not limited thereto.

To give a more specific example, modification of valine aminoacyl-tRNA synthetase (ValRS) includes, for example, modification of amino acid(s) at position 43 and/or position 45 and/or position 279 of SEQ ID NO: 24 (natural ValRS), or amino acids at positions corresponding to these positions. Amino acids selected for substitution are not limited, but as mentioned above, for example, Thr can be substituted with Ser, Ala, or Gly, and amino acids (e.g., Asn) other than Thr can be substituted with Gly or Ala. For example, preferred are substitution of the amino acid at position 43 of SEQ ID NO: 24 or an amino acid at a position corresponding to position 43 of SEQ ID NO: 24 with Gly or Ala, and/or substitution of the amino acid at position 45 of SEQ ID NO: 24 or an amino acid at the position corresponding to position 45 of SEQ ID NO: 24 with Ser, and/or substitution of the amino acid at position 279 or an amino acid at a position corresponding to position 279 with Gly or Ala. These substitutions may be any one of the substitutions, a combination of any of these substitutions (e.g., substitutions at position 43 and position 45, substitutions at position 43 and position 279, or substitutions at position 45 and position 279), or all of the substitutions. Other substitutions may be further combined. As a more specific illustration, N43 and/or T45 and/or T279 of SEQ ID NO: 24, or amino acids at the positions corresponding to these, are preferably substituted, and preferably substituted to N43G and/or T45S and/or T279A.

Modification of serine aminoacyl-tRNA synthetase (SerRS) includes, for example, modification of amino acid(s) at position 237 and/or position 239 of SEQ ID NO: 26 (natural SerRS), or amino acids at the positions corresponding to these positions. Amino acids selected for substitution are not limited, but as mentioned above, for example, Thr can be substituted with Ser, and amino acids (e.g., Glu) other than Thr can be substituted with Gly or Ala. For example, preferred are substitution of the amino acid at position 237 of SEQ ID NO: 26 or an amino acid at the position corresponding to position 237 of SEQ ID NO: 26 with Ser, and/or substitution of the amino acid at position 239 of SEQ ID NO: 26 or an amino acid at the position corresponding to position 239 of SEQ ID NO: 26 with Gly or Ala. These substitutions may be any one or both of the substitutions. Other substitutions may be further combined. As a more specific illustration, T237 and/or E239 of SEQ ID NO: 26, or amino acids at positions corresponding to these, are preferably substituted, and preferably substituted to T237S and/or E239G (or E239A).

Modification of phenylalanine aminoacyl-tRNA synthetase α subunit (PheRS α) includes, for example, modification of amino acid at position 169 of SEQ ID NO: 28 (natural PheRS α subunit), or an amino acid at a position corresponding to position 169 of SEQ ID NO: 28. Amino acids selected for substitution are not limited, but as mentioned above, for example, Thr can be substituted with Ser, and amino acids other than Thr can be substituted with glycine (Gly) or alanine (Ala) (more preferably Gly). For example, preferred is substitution of the amino acid at position 169 of SEQ ID NO: 28 or an amino acid at the position corresponding to position 169 of SEQ ID NO: 28 with Gly. Other substitutions may be further combined. As a more specific illustration, Q169 of SEQ ID NO: 28, or an amino acid at the position corresponding to Q169 of SEQ ID NO: 28, is preferably substituted, and preferably substituted to Q169G (or Q169A).

Modification of threonine aminoacyl-tRNA synthetase (ThrRS) includes, for example, modification of amino acid(s) at position 332 and/or position 511 of SEQ ID NO: 29 (natural ThrRS), or amino acids at positions corresponding to these positions. Amino acids selected for substitution are not limited, but as mentioned above, for example, Thr can be substituted with Ser, and amino acids (e.g., Met and His) other than Thr can be substituted with Gly. For example, preferred is/are substitution of the amino acid at position 332 of SEQ ID NO: 29 or an amino acid at a position corresponding to position 332 of SEQ ID NO: 29 with Gly and/or substitution of the amino acid at position 511 of SEQ ID NO: 29 or an amino acid at a position corresponding to position 511 of SEQ ID NO: 29 with Gly. These substitutions may be any one or both of the substitutions. Other substitutions may be further combined. As a more specific illustration, M332 and/or H511 of SEQ ID NO: 29, or amino acids at positions corresponding to these, are preferably substituted, and preferably substituted to M332G and/or H511G.

Modification of tryptophan aminoacyl-tRNA synthetase (TrpRS) includes, for example, modification of amino acid(s) at position 132 and/or position 150 and/or position 153 of SEQ ID NO: 188 (natural TrpRS), or amino acids at a position corresponding to these positions. Amino acids selected for substitution are not limited, but as mentioned above, for example, Met can be substituted with Val or Ala, and amino acids (e.g., Gln) other than Met can be substituted with Ala. For example, preferred is/are substitution of the amino acid at position 132 of SEQ ID NO: 188 or an amino acid at a position corresponding to position 132 of SEQ ID NO: 188 with Val or Ala, and/or substitution of the amino acid at position 150 of SEQ ID NO: 188 or an amino acid at a position corresponding to position 150 of SEQ ID NO: 188 with Ala, and/or substitution of the amino acid at position 153 or an amino acid at a position corresponding to position 153 with Ala. These substitutions may be any one of the substitutions, a combination of any of these substitutions (e.g., substitutions at position 132 and position 150, substitutions at position 132 and position 153, or substitutions at position 150 and position 153), or all of the substitutions. Other substitutions may be further combined. As a more specific illustration, M132 and/or Q150 and/or H153 of SEQ ID NO: 188, or amino acids at positions corresponding to these, are preferably substituted, and preferably substituted to M132V and/or Q150A and/or H153A.

Modification of leucine aminoacyl-tRNA synthetase (LeuRS) includes, for example, modification of the amino acid at position 43 of SEQ ID NO: 189 (natural LeuRS), or an amino acid at a position corresponding to position 43 of SEQ ID NO: 189. Amino acids selected for substitution are not limited, but as mentioned above, for example, Thr can be substituted with Gly. For example, preferred is substitution of the amino acid at position 43 of SEQ ID NO: 189 or an amino acid at a position corresponding to position 43 of SEQ ID NO: 189 with Gly. Other substitutions may be further combined. As a more specific illustration, Y43 of SEQ ID NO: 189, or an amino acid at a position corresponding to Y43 of SEQ ID NO: 189, is preferably substituted, and preferably substituted to Y43G.

A method for producing a mutant N-methyl aminoacyl-tRNA synthetase, which is modified by substituting an amino acid at a specific position with another amino acid, according to the present invention, can be performed using any known genetic engineering technique. For example, DNA fragments having base sequences encoding amino acid sequences comprising amino acids at positions of interest are amplified using primers having base sequences substituted with base sequences encoding amino acid sequences comprising modified amino acids, resulting in base sequences encoding amino acid sequences comprising modified amino acids. The amplified DNA fragments are ligated together to obtain a full-length DNA encoding the mutant aminoacyl-tRNA synthetase. This full-length DNA can be expressed using a host cell such as E. coli to easily produce the mutant N-methyl aminoacyl-tRNA synthetase. Primers used in the method are 20 to 70 bases in length, and preferably about 20 to 50 bases in length. The primers have 1 to 3 base mismatches with the original unmodified base sequence, and therefore relatively long primers, for example, primers of 20 bases or more in length are preferably used.

A method for producing a mutant N-methyl aminoacyl-tRNA synthetase, which is modified by substituting an amino acid at a specific position with another amino acid, according to the present invention, is not limited to the method as described above, and various genetic engineering techniques, such as known point mutation techniques and gene synthesis techniques, and methods for introducing modified fragments using restriction enzymes, can be utilized. Expression hosts are not limited to E. coli, and animal cells and cell-free translation systems may also be used.

Modified ARSs according to the present invention include a polypeptide comprising an amino acid sequence set forth in any of SEQ ID NOs: 1-11 and 182-187 (PheRS05, PheRS04, ValRS04, ValRS13, ValRS13-11, SerRS03, SerRS05, SerRS35, SerRS37, ThrRS03, ThrRS14, ValRS66, ValRS67, TrpRS04, TrpRS05, TrpRS18, and LeuRS02) and polypeptides functionally equivalent to the polypeptide. “Functionally equivalent polypeptides” are ARSs with a high structural identity to a polypeptide comprising an amino acid sequence set forth in any of SEQ ID NOs: 1-11 and 182-187 and have reactivity with N-methyl amino acids. More specifically, “functionally equivalent polypeptides” are polypeptides that have amino acids modified according to the above description in ARSs with a high structural identity to a polypeptide comprising an amino acid sequence set forth in any of SEQ ID NOs: 1-11 and 182-187, thereby having increased reactivity with N-methyl amino acids compared to unmodified ARSs. Increased reactivity with N-methyl amino acids may be, for example, increased substrate specificity to N-methyl amino acids (e.g., increased value of reactivity with N-methyl amino acids/reactivity with unmodified amino acids).

Such polypeptides include, for example, a polypeptide in which one or more amino acids (preferably 1 to 20 amino acids, for example, 1 to 10 amino acids, 1 to 7 amino acids, 1 to 5 amino acids, 1 to 3 amino acids, 1 to 2 amino acids, or 1 amino acid) are substituted, deleted, inserted, and/or added in an amino acid sequence set forth in any of SEQ ID NOs: 1-11 and 182-187. Such polypeptides may also be, for example, a polypeptide in which one to several amino acids are substituted, deleted, inserted, and/or added in an amino acid sequence set forth in any of SEQ ID NOs: 1-11 and 182-187.

A polypeptide functionally equivalent to a modified ARS comprising an amino acid sequence set forth in any of SEQ ID NOs: 1-11 and 182-187 typically has a high identity to an amino acid sequence set forth in any of SEQ ID NOs: 1-11 and 182-187. A polynucleotide encoding a functionally equivalent polypeptide also typically has a high identity to a base sequence (e.g., SEQ ID NOs: 12-22 and 190-195) encoding an amino acid sequence set forth in any of SEQ ID NOs: 1-11 and 182-187. High identity (sequence identity) specifically refers to 70% or more, preferably 80% or more, 85% or more, 90% or more, 95% or more, 97% or more, 98% or more, or 99% or more identity.

The identity of amino acid sequences or base sequences can be determined using the BLAST algorithm by Karlin and Altschul (Proc. Natl. Acad. Sci. USA (1993) 90: 5873-7). Programs called BLASTN and BLASTX have been developed based on the algorithm (Altschul et al., J. Mol. Biol. (1990) 215: 403-10). When a base sequence is analyzed using BLAST-based BLASTN, parameters are set as, for example, score=100 and wordlength=12. When an amino acid sequence is analyzed using BLAST-based BLASTX, parameters are set as, for example, score=50 and wordlength=3. When BLAST and Gapped BLAST programs are used, default parameters of each program are used. Specific procedures of these analysis methods are known (see, information at the website of Basic Local Alignment Search Tool (BLAST) in National Center for Biotechnology Information (NCBI).

The polypeptides according to the present invention include any polypeptide of the following (a) to (d), wherein the amino acid corresponding to position 43 of SEQ ID NO: 24 (ValRS) is other than Asn, and/or the amino acid corresponding to position 45 of SEQ ID NO: 24 (ValRS) is other than Thr and/or the amino acid corresponding to position 279 of SEQ ID NO: 24 (ValRS) is other than Thr (i.e., a polypeptide in which the amino acid at at least one of these 3 positions is other than the indicated respective amino acid) and wherein the polypeptide has increased reactivity to N-methyl Val compared to a polypeptide in which the amino acids corresponding to position 43, position 45, and position 279 are Asn, Thr, and Thr, respectively:
(a) a polypeptide comprising an amino acid sequence having a high identity to an amino acid sequence set forth in any of SEQ ID NOs: 3-5, 182, and 183 (ValRS04, ValRS13, ValRS13-11, ValRS66, and ValRS67);
(b) a polypeptide comprising an amino acid sequence in which one or more amino acids are substituted, deleted, inserted, and/or added in an amino acid sequence set forth in any one of SEQ ID NOs: 3-5, 182, and 183;
(c) a polypeptide encoded by a base sequence with high identity to a base sequence set forth in any one of SEQ ID NOs: 14-16, 190, and 191 (DNA encoding ValRS04, ValRS13, ValRS13-11, ValRS66, and ValRS67); or
(d) a polypeptide encoded by a DNA fragment hybridizing with a strand complementary to a base sequence set forth in any one of SEQ ID NOs: 14-16, 190, and 191 under stringent conditions.

The above-mentioned polypeptides preferably have (i) Gly or Ala at the amino acid position corresponding to position 43 and/or (ii) Ser at the amino acid position corresponding to position 45 and/or (iii) Gly or Ala at the amino acid position corresponding to position 279. Such polypeptides include natural polypeptides and artificially-modified polypeptides, and preferably include polypeptides in which (i) the amino acid at the position corresponding to position 43 is substituted with Gly or Ala and/or (ii) the amino acid at the position corresponding to position 45 is substituted with Ser and/or (iii) the amino acid at the position corresponding to position 279 is substituted with Gly or Ala.

The polypeptides according to the present invention include any polypeptide of the following (a) to (d), wherein the amino acid corresponding to position 237 of SEQ ID NO: 26 (SerRS) is other than Thr and/or the amino acid corresponding to position 239 of SEQ ID NO: 26 (SerRS) is other than Glu (i.e., a polypeptide in which the amino acid at at least one of these 2 positions is other than the indicated respective amino acid) and wherein the polypeptide has increased reactivity with N-methyl Ser compared to a polypeptide in which the amino acids corresponding to position 237 and position 239 are Thr and Glu respectively:
(a) a polypeptide comprising an amino acid sequence having a high identity to an amino acid sequence set forth in any one of SEQ ID NOs: 6-9 (SerRS03, SerRS05, SerRS35, and SerRS37);
(b) a polypeptide comprising an amino acid sequence in which one or more amino acids are substituted, deleted, inserted, and/or added in an amino acid sequence set forth in any one of SEQ ID NOs: 6-9;
(c) a polypeptide encoded by a base sequence having a high identity to a base sequence set forth in any one of SEQ ID NOs: 17-20 (DNA encoding SerRS03, SerRS05, SerRS35, and SerRS37); or
(d) a polypeptide encoded by a DNA fragment hybridizing with a strand complementary to a base sequence set forth in any one of SEQ ID NOs: 17-20 under stringent conditions.

The above-mentioned polypeptides preferably have (i) Ser at the amino acid position corresponding to position 237 and/or (ii) Gly or Ala at the amino acid position corresponding to position 239. Such polypeptides include natural polypeptides and artificially-modified polypeptides, and preferably include polypeptides in which (i) the amino acid at the position corresponding to position 237 is substituted with Ser and/or (ii) the amino acid at the position corresponding to position 239 is substituted with Gly or Ala.

Such polypeptides according to the present invention include any polypeptide of the following (a) to (d), wherein the polypeptide has any amino acid other than Gln at the position corresponding to position 169 of SEQ ID NO: 28 (PheRS) and has increased reactivity with N-methyl Phe compared to a polypeptide in which the amino acid at position 169 of SEQ ID NO: 28 (PheRS) is Gln:
(a) a polypeptide comprising an amino acid sequence having a high identity to an amino acid sequence set forth in any one of SEQ ID NOs: 1-2 (PheRS05 and PheRS04);
(b) a polypeptide comprising an amino acid sequence in which one or more amino acids are substituted, deleted, inserted, and/or added in an amino acid sequence set forth in any one of SEQ ID NOs: 1-2;
(c) a polypeptide encoded by a base sequence having a high identity to a base sequence set forth in any one of SEQ ID NOs: 12-13 (DNA encoding PheRS05 and PheRS04); or
(d) a polypeptide encoded by a DNA fragment hybridizing with a strand complementary to a base sequence set forth in any one of SEQ ID NOs: 12-13 under stringent conditions.

The above-mentioned polypeptides preferably have Gly or Ala at the amino acid position corresponding to position 169. Such polypeptides include natural polypeptides and artificially-modified polypeptides, and preferably include polypeptides in which the amino acid at the position corresponding to position 169 is substituted with Gly or Ala.

The polypeptides according to the present invention include any polypeptide of the following (a) to (d), wherein the amino acid corresponding to position 332 of SEQ ID NO: 29 (ThrRS) is other than Met and/or the amino acid corresponding to position 511 of SEQ ID NO: 29 (ThrRS) is other than His (i.e., a polypeptide in which the amino acid at at least one of these 2 positions is other than the indicated respective amino acid) and wherein the polypeptide has increased reactivity with N-methyl Thr compared to a polypeptide in which the amino acids corresponding to position 332 and position 511 are Met and His, respectively:
(a) a polypeptide comprising an amino acid sequence having a high identity to an amino acid sequence set forth in any one of SEQ ID NOs: 10-11 (ThrRS03 and ThrRS14);
(b) a polypeptide comprising an amino acid sequence in which one or more amino acids are substituted, deleted, inserted, and/or added in an amino acid sequence set forth in any one of SEQ ID NOs: 10-11;
(c) a polypeptide encoded by a base sequence having a high identity to a base sequence set forth in any one of SEQ ID NOs: 21-22 (DNA encoding ThrRS03 and ThrRS14); or
(d) a polypeptide encoded by a DNA fragment hybridizing with a strand complementary to a nucleotide sequence set forth in any one of SEQ ID NOs: 21-22 under stringent conditions.

The above-mentioned polypeptides preferably have (i) Gly at the amino acid position corresponding to 332 and/or (ii) Gly at the amino acid position corresponding to position 511. Such polypeptides include natural polypeptides and artificially-modified polypeptides, and preferably include polypeptides in which the amino acid at the position corresponding to position 332 is substituted with Gly and/or the amino acid at the position corresponding to position 511 is substituted with Gly.

The polypeptides according to the present invention include a polypeptide that is any polypeptide of the following (a) to (d), wherein the amino acid corresponding to position 132 of SEQ ID NO: 188 (TrpRS) is other than Met and/or the amino acid corresponding to position 150 of SEQ ID NO: 188 (TrpRS) is other than Gln and/or the amino acid corresponding to position 153 of SEQ ID NO: 188 (TrpRS) is other than His (i.e., a polypeptide in which the amino acid at at least one of these 3 positions is other than the indicated respective amino acid) and wherein the polypeptide has increased reactivity with N-methyl Trp compared to a polypeptide in which the amino acids corresponding to position 132, position 150, and position 153 are Met, Gln, and His, respectively:
(a) a polypeptide comprising an amino acid sequence having a high identity to an amino acid sequence set forth in any one of SEQ ID NOs: 184-186 (TrpRS04, TrpRS05, and TrpRS18);
(b) a polypeptide comprising an amino acid sequence in which one or more amino acids are substituted, deleted, inserted, and/or added in an amino acid sequence set forth in any one of SEQ ID NOs: 184-186;
(c) a polypeptide encoded by a base sequence having a high identity to a base sequence set forth in any one of SEQ ID NOs: 192-194 (DNA encoding TrpRS04, TrpRS05, and TrpRS18); or
(d) a polypeptide encoded by a DNA fragment hybridizing with a strand complementary to a base sequence set forth in any one of SEQ ID NOs: 192-194 under stringent conditions.

The above-mentioned polypeptides preferably have (i) Val or Ala at the amino acid position corresponding to position 132 and/or (ii) Ala at the amino acid position corresponding to position 150 and/or (iii) Ala at the amino acid position corresponding to position 153. Such polypeptides include natural polypeptides and artificially-modified polypeptides, and preferably include polypeptides in which (i) the amino acid at the position corresponding to position 132 is substituted with Val or Ala and/or (ii) the amino acid at the position corresponding to position 150 is substituted with Ala and/or (iii) the amino acid at the position corresponding to position 153 is substituted with Ala.

The polypeptides according to the present invention include any polypeptide of the following (a) to (d), wherein the polypeptide has any amino acid other than Tyr at the position corresponding to position 43 of SEQ ID NO: 189 (LeuRS) and has increased reactivity with N-methyl Leu compared to a polypeptide in which the amino acid corresponding to position 43 is Tyr:
(a) a polypeptide comprising an amino acid sequence having a high identity to the amino acid sequence set forth in SEQ ID NO: 187 (LeuRS02);
(b) a polypeptide comprising an amino acid sequence in which one or more amino acids are substituted, deleted, inserted, and/or added in an amino acid sequence set forth in any one of SEQ ID NO: 187;
(c) a polypeptide encoded by a base sequence having a high identity to the base sequence set forth in SEQ ID NO: 195 (DNA encoding LeuRS02); or
(d) a polypeptide encoded by a DNA fragment hybridizing with a strand complementary to the base sequence set forth in SEQ ID NO: 195 under stringent conditions.

The above-mentioned polypeptides preferably have Gly at the amino acid position corresponding to position 43. Such polypeptides include natural polypeptides and artificially-modified polypeptides, and preferably include polypeptides in which the amino acid at the position corresponding to position 43 is substituted with Gly.

In the case of polypeptides obtained by modifying natural ARSs, polypeptides according to the present invention are those with increased reactivity to N-methyl amino acids compared to unmodified ARSs. When the polypeptides are natural ARSs or artificially produced polypeptides, the polypeptides according to the present invention are those with reactivity to N-methyl amino acids, namely those having an activity to acylate tRNAs with N-methyl amino acids. When comparing the reactivities of modified ARSs and natural ARSs, and the ARSs are made up of multiple subunits, subunit(s) other than that/those compared is/are the same is/are used. These subunits may be natural (or wild-type) or a modified as long as they are the same in both ARSs.

High identity (or high sequence identity) refers to, as mentioned above, for example, 70% or more, preferably 80% or more, 85% or more, 90% or more, 95% or more, 97% or more, 98% or more, or 99% or more identity. The number of amino acids substituted, deleted, inserted, and/or added may be one or several, for example, 1 to 20, preferably 1 to 15, more preferably 1 to 10, more preferably 1 to 8, more preferably 1 to 7, more preferably 1 to 6, more preferably 1 to 5, more preferably 1 to 4, more preferably 1 to 3, more preferably 1 to 2, and more preferably 1. Stringent hybridization conditions refer to, for example, conditions of about “1×SSC, 0.1% SDS, at 37° C.”, more strictly conditions of about “0.5×SSC, 0.1% SDS, at 42° C.”, further more strictly conditions of about “0.2×SSC, 0.1% SDS, at 65° C.”, and yet more strictly conditions of about “0.1×SSC, 0.1% SDS, at 65° C.”. It is noted that the conditions of SSC, SDS, and temperature as described above are only examples of combinations. Those skilled in the art can achieve stringency similar to those described above by appropriately combining the above-mentioned or other factors that determine hybridization stringency (such as probe concentration, probe length, and duration of hybridization).

The present invention also relates to polynucleotides encoding the polypeptides according to the present invention. The polynucleotides according to the present invention comprise any polynucleotide as long as it comprises a sequence encoding a polypeptide according to the present invention as described above. The polynucleotides according to the present invention also comprise any one of genomic DNA, cDNA, and DNAs artificially produced based on the genomic DNA or cDNA. Genomic DNA comprises exons and introns. That is to say, genomic DNA may or may not comprise introns, and may or may not comprise untranslated regions (5′ UTR and/or 3′ UTR), transcription control elements, and the like. cDNA is a nucleic acid sequence derived from a portion of intronic sequence and may comprise a nucleic acid sequence encoding an amino acid sequence.

The polynucleotides also comprise degenerate polynucleotides comprising any codon that encodes the same amino acid. The polynucleotides according to the present invention may also be a polynucleotide derived from desired organisms.

The polynucleotides according to the present invention may be obtained using any method. For example, a complementary DNA (cDNA) prepared from mRNA, a DNA prepared from genomic DNA, a DNA obtained by chemical synthesis, a DNA obtained by amplifying RNA or DNA as a template using PCR, and a DNA constructed by appropriately combining these techniques are all included. The polynucleotides according to the present invention can be produced by cloning the genomic DNA or RNA encoding the polypeptide according to the present invention according to conventional methods and introducing mutations into the cloned genomic DNA or RNA.

For example, in a method for cloning cDNA from mRNA encoding a polypeptide according to the present invention, first, the mRNA encoding the polypeptide according to the present invention is prepared according to conventional methods from any tissue or cell where the polypeptide according to the present invention is expressed and produced. For example, total RNA prepared using a method such as the guanidine thiocyanate method, hot-phenol method, or AGPC method can be subjected to affinity chromatography on oligo (dT) cellulose, poly U-Sepharose, or the like.

The obtained mRNA is then used as a template to synthesize a cDNA strand by using a known method (Mol. Cell. Biol., Vol. 2, p. 161, 1982; Mol. Cell. Biol., Vol. 3, p. 280, 1983; Gene, Vol. 25, p. 263, 1983), for example, by using reverse transcriptase. The cDNA strand is converted into a double-stranded cDNA and incorporated into a plasmid vector, phage vector, cosmid vector, or the like. The vector is used to transform E. coli or to perform in vitro packaging followed by transfection of E. coli to generate a cDNA library.

The cDNA library can be screened using a polynucleotide according to the present invention (e.g., SEQ ID NOs: 12-22, 190-195) or a portion thereof as a probe to obtain a gene of interest. Alternatively, the cDNA library can be directly amplified by PCR using a polynucleotide according to the present invention (e.g., SEQ ID NOs: 12-22, 190-195) or a portion thereof as a primer. The sites and lengths of probes and primers may be appropriately determined.

The present invention also relates to vectors (recombinant vectors) comprising polynucleotides encoding the polypeptides according to the present invention as described above. The vectors according to the present invention are not particularly limited as long as they can replicate and can be maintained or can self-proliferate in any host prokaryotic and/or eukaryotic cell. The vectors include plasmid vectors, phage vectors, viral vectors, and the like.

Examples of vectors for cloning include, for example, pUC19, λgt10, λgt11, and the like. Furthermore, the vectors preferably have a promoter that can express the polypeptide according to the present invention when cells that can express the polynucleotide in host cells are isolated.

The recombinant vectors according to the present invention can be prepared by simply ligating a polynucleotide encoding a polypeptide according to the present invention into a vector for recombination (a plasmid DNA and bacteriophage DNA) available in the art according to conventional methods.

Examples of recombinant vectors that can be used include, for example, plasmids from E. coli (such as pBR322, pBR325, pUC12, pUC13, and pUC19), plasmids from yeast (such as pSH19 and pSH15), and plasmids from Bacillus subtilis (such as pUB110, pTP5, and pC194).

Examples of phages include bacteriophage such as λ phage, and further animal and insect viruses such as retrovirus, vaccinia virus, nucleopolyhedrovirus, and lentivirus (pVL1393, from Invitrogen).

Expression vectors are useful for expressing polynucleotides encoding the polypeptides according to the present invention and production of the polypeptides according to the present invention. The expression vectors are not particularly limited as long as they have functions to express polynucleotides encoding the polypeptides according to the present invention and produce the polypeptides in any host prokaryotic and/or eukaryotic cell.

For example, expression vectors include pMAL C2, pEF-BOS (Nucleic Acid Research, Vol. 18, 1990, p. 5322, and the like), or pME18S (“Idenshi Kougaku Handbook (Genetic Engineering Handbook)”, supplementary volume of Jikken Igaku (Experimental Medicine), 1992, and the like).

The present invention also relates to fusion between polypeptides according to the present invention with another protein/other proteins. A fusion polypeptide according to the present invention is a fusion polypeptide between a polypeptide having reactivity with N-methyl amino acids according to the present invention and another polypeptide. The fusion polypeptide itself may have no reactivity with N-methyl amino acids as long as it comprises a polypeptide chain having reactivity with N-methyl amino acids according to the present invention. When prepared as a fusion protein with, for example, Glutathione S-transferase (GST), the fusion polypeptide according to the present invention can be prepared by subcloning a cDNA encoding the polypeptide according to the present invention into, for example, plasmid pGEX4T1 (from Pharmacia), transforming E. coli DH5α or the like with the plasmid, and culturing the transformant.

Alternatively, a fusion polypeptide according to the present invention can be prepared as a fusion with HA (influenza agglutinin), immunoglobulin constant region, β-galactosidase, maltose-binding protein (MBP), or the like. Moreover, a fusion polypeptide can be prepared as a fusion with, for example, any known peptide such as FLAG (Hopp, T. P. et al., BioTechnology (1988) 6, 1204-1210), a tag consisting of several (e.g., six) histidine (His) residues (such as 6×His, 10×His), influenza agglutinin (HA), a human c-myc fragment, a VSV-GP fragment, a p18HIV fragment, T7-tag, HSV-tag, E-tag, a SV40T antigen fragment, lck tag, an α-tubulin fragment, B-tag, a Protein C fragment, Stag, StrepTag, and HaloTag.

The vectors according to the present invention preferably comprise at least a promoter-operator region, the initiation codon, a polynucleotide encoding the polypeptide according to the present invention, a termination codon, a terminator region, and a replicable unit when bacteria, particularly E. coli, are used as host cells.

The expression vectors preferably comprise at least a promoter, the initiation codon, a polynucleotide encoding a polypeptide according to the present invention, and a termination codon when yeast, animal cells, or insect cells are used as hosts.

The vectors may also comprise DNA encoding a signal peptide, an enhancer sequence, 5′ and 3′ untranslated regions of the gene encoding a polypeptide according to the present invention, a splicing junction, a polyadenylation site, a selection marker region, a replicable unit, or the like.

The vectors may also comprise, if desired, a marker gene (such as a gene amplification gene, a drug-resistant gene) that allows selection of hosts in which gene amplification and transformation have been achieved.

Examples of marker genes include, for example, dihydrofolate reductase (DHFR) gene, thymidine kinase gene, neomycin resistant gene, glutamic acid synthetase gene, adenosine deaminase gene, ornithine decarboxylase gene, hygromycin B phosphotransferase gene, aspartate transcarbamylase gene, and the like.

The promoter-operator region for expressing a polypeptide according to the present invention in bacteria can include, a promoter, an operator, and Shine-Dalgarno (SD) sequence (such as AAGG).

An example of a promoter-operator region includes one comprising, for example, Trp promoter, lac promoter, recA promoter, αPL promoter, lpp promoter, tac promoter, or the like when the host is a bacterium of the genus Escherichia, for example.

Promoters for expressing a polypeptide according to the present invention in yeast include PH05 promoter, PGK promoter, GAP promoter, and ADH promoter.

The promoters include SL01 promoter, SP02 promoter, penP promoter, and the like when the host is a bacterium of the genus Bacillus.

The promoters also include promoters derived from SV40, retroviral promoters, heat shock promoters, and the like when the hosts are eukaryotic cells such as mammalian cells. The promoters are preferably those derived from SV40 and retroviral promoters. However, the promoters are not particularly limited to those described above. Also, enhancers can be effectively used for expression.

An example of a suitable initiation codon includes the methionine codon (ATG). Examples of termination codons include common termination codons (e.g., TAG, TGA, and TAA). Terminator regions that can be used include natural or synthetic terminators generally used.

The replicable unit refers to DNA having an ability to replicate the full DNA sequence of the replicable unit in host cells, and includes natural plasmids, artificially modified plasmids (DNA fragments prepared from natural plasmids), synthetic plasmids, and the like. Suitable plasmids include plasmid pBR322 or an artificially modified pBR322 (a DNA fragment obtained by digesting pBR322 with a suitable restriction enzyme) for E. coli; yeast 2μ plasmid or yeast chromosomal DNA for yeast; and plasmid pRSVneo (ATCC 37198), plasmid pSV2dhfr (ATCC 37145), plasmid pdBPV-MMTneo (ATCC 37224), plasmid pSV2neo (ATCC 37149), and the like for mammalian cells.

Enhancer sequence, polyadenylation site, and splicing junction that can be used are those commonly used by those skilled in the art, such as for example those derived from SV40.

The expression vectors according to the present invention can be prepared by ligating at least a promoter as described above, an initiation codon, a polynucleotide encoding a polypeptide according to the present invention, a termination codon, and a terminator region to a suitable replicable unit continuously and circularly. Suitable DNA fragments (e.g., a linker, other restriction enzyme cleavage sites, and the like) can also be used in conventional techniques such as digestion with restriction enzymes and ligation with T4 DNA ligase, if desired.

The present invention also relates to recombinant cells transformed with the vectors according to the present invention as described above, and the recombinant cells according to the present invention can be prepared by introducing the expression vectors as described above into host cells.

Host cells used in the present invention are not particularly limited as long as they are compatible with the expression vectors described above and can be transformed. Examples of host cells include various cells including natural cells and artificially-established recombinant cells commonly used in the technical field of the present invention, for example, bacteria (Escherichia bacteria, Bacillus bacteria), yeast (such as Saccharomyces, Pichia), animal cells, insect cells, and the like.

The host cells are preferably E. coli or animal cells, and examples include, for example, E. coli (such as DH5α, TB1, HB101), mouse-derived cells (such as COP, L, C127, Sp2/0, NS-1, or NIH3T3), rat-derived cells (PC12, PC12 h), hamster-derived cells (such as BHK and CHO), monkey-derived cells (such as COS1, COS3, COS7, CV1, and Velo), human-derived cells (such as Hela, diploid fibroblast-derived cells, myeloma cells, and HepG2), and the like.

Introduction of expression vectors into host cells (transformation (transfection)) can be performed according to conventional methods ([for E. coli, Bacillus subtilis, and the like]: Proc. Natl. Acad. Sci. USA., Vol. 69, p. 2110, 1972; Mol. Gen. Genet., Vol. 168, p. 111, 1979; J. Mol. Biol., Vol. 56, p. 209, 1971; [for Saccharomyces cerevisiae]: Proc. Natl. Acad. Sci. USA., Vol. 75, p. 1927, 1978; J. Bacteriol., Vol. 153, p. 163, 1983); [for animal cells]: Virology, Vol. 52, p. 456, 1973; [for insect cells]: Mol. Cell. Biol., Vol. 3, p. 2156-2165, 1983).

The polypeptides according to the present invention can be produced by culturing recombinant transformed cells including expression vectors prepared as described above (hereinafter used to mean inclusion of an inclusion body) in a nutrient medium according to any conventional method.

The polypeptides according to the present invention can be produced such as by culturing recombinant cells as described above, particularly animal cells and allowing the recombinant cells to secrete the polypeptides into culture supernatant.

The obtained culture is subjected to filtration, centrifugation, or any other similar technique to obtain a culture filtrate (supernatant). The polypeptides according to the present invention are purified and isolated from the culture filtrate according to any conventional method commonly used to purify and isolate natural or synthetic proteins.

Isolation and purification methods include, for example, methods utilizing solubility such as salt precipitation and solvent precipitation, dialysis, ultrafiltration, gel filtration, methods utilizing the difference in molecular weight such as sodium dodecyl sulphate-polyacrylamide gel electrophoresis, methods utilizing electric charge such as ion exchange chromatography and hydroxylapatite chromatography, methods utilizing specific affinity such as affinity chromatography, methods utilizing the difference in hydrophobicity such as reversed-phase high-performance liquid chromatography, methods utilizing the difference in isoelectric point such as isoelectric focusing, and the like.

Meanwhile, when a polypeptide according to the present invention is present in periplasm or cytoplasm of cultured recombinant cells (e.g., E. coli), the culture is subjected to any conventional method such as filtration or centrifugation to collect bacterial pellets or cells. The collected bacterial pellets or cells are suspended in a suitable buffer, and cell wall and/or cytoplasmic membrane is/are disrupted by any method such as for example sonication, lysozyme, and freeze-thawing. Any method such as centrifugation or filtration is then performed to obtain membrane fraction containing the protein according to the present invention. The membrane fraction is solubilized with any surfactant such as Triton™-X100 to give a crude solution. Then, the crude solution can be isolated and purified using any conventional method as described previously.

The present invention also relates to a polynucleotide (cDNA or genomic DNA) encoding a polypeptide according to the present invention as described above or an oligonucleotide hybridizing with a strand complementary to the polynucleotide. For example, the oligonucleotide is a base sequence at least comprising modified sites of ARSs according to the present invention or an oligonucleotide hybridizing with a strand complementary to the base sequence. An oligonucleotide according to the present invention is also a partial fragment of polynucleotides encoding a modified ARS according to the present invention, and is preferably a fragment comprising bases in modified sites of the modified ARSs or a strand complementary to the fragment.

For example, for a polynucleotide encoding a polypeptide according to the present invention having reactivity with N-methyl Val, the oligonucleotide according to the present invention may be an oligonucleotide comprising bases encoding the codon(s) for an amino acid corresponding to position 43, and/or an amino acid corresponding to position 45, and/or an amino acid corresponding to position 279 of the amino acid sequences set forth in SEQ ID NOs: 3-5, 182, and 183 (ValRS04, ValRS13, ValRS13-11, ValRS66, and ValRS67), or an oligonucleotide consisting of a sequence complementary to the oligonucleotide. In this case, an amino acid corresponding to position 43 is preferably Gly or Ala (more preferably Gly), and an amino acid corresponding to position 45 is preferably Ser, and an amino acid corresponding to position 279 is preferably Gly or Ala (more preferably Ala).

For example, for a polynucleotide encoding a polypeptide according to the present invention having reactivity with N-methyl Ser, the oligonucleotide according to the present invention may be an oligonucleotide comprising bases encoding the codon(s) for an amino acid corresponding to position 237 and/or an amino acid corresponding to position 239 of the amino acid sequences set forth in SEQ ID NOs: 6-9 (SerRS03, SerRS05, SerRS35, and SerRS37), or an oligonucleotide consisting of a sequence complementary to the oligonucleotide. In this case, an amino acid corresponding to position 237 is preferably Ser, and an amino acid corresponding to position 239 is preferably Gly or Ala (more preferably Gly).

For example, for a polynucleotide encoding a polypeptide according to the present invention having reactivity with N-methyl Phe, the oligonucleotide according to the present invention may be an oligonucleotide comprising bases encoding the codon for an amino acid corresponding to position 169 of the amino acid sequences set forth in SEQ ID NOs: 1-2 (PheRS04 and PheRS05) or an oligonucleotide consisting of a sequence complementary to the oligonucleotide. In this case, an amino acid corresponding to position 169 is preferably Gly or Ala.

For example, for a polynucleotide encoding a polypeptide according to the present invention having reactivity with N-methyl Thr, the oligonucleotide according to the present invention may be an oligonucleotide comprising bases encoding the codon(s) for an amino acid corresponding to position 332 and/or an amino acid corresponding to position 511 of the amino acid sequences set forth in SEQ ID NOs: 10-11 (ThrRS03 and ThrRS14), or an oligonucleotide consisting of a sequence complementary to the oligonucleotide. In this case, an amino acid corresponding to position 332 is preferably Gly, and an amino acid corresponding to position 511 is preferably Gly.

For example, for a polynucleotide encoding a polypeptide according to the present invention having reactivity with N-methyl Trp, the oligonucleotide according to the present invention may be an oligonucleotide comprising bases encoding the codon(s) for an amino acid corresponding to position 132, and/or an amino acid corresponding to position 150, and/or an amino acid corresponding to position 153 of the amino acid sequences set forth in SEQ ID NOs: 184-186 (TrpRS04, TrpRS05, and TrpRS18), or an oligonucleotide consisting of a sequence complementary to the oligonucleotide. In this case, an amino acid corresponding to position 132 is preferably Val or Ala (more preferably Val), an amino acid corresponding to position 150 is preferably Ala, and an amino acid corresponding to position 279 is preferably Ala.

For example, for a polynucleotide encoding a polypeptide according to the present invention having reactivity with N-methyl Leu, the oligonucleotide according to the present invention may be an oligonucleotide comprising bases encoding the codon for an amino acid corresponding to position 43 of the amino acid sequence set forth in SEQ ID NO: 187 (LeuRS02) or an oligonucleotide consisting of a sequence complementary to the oligonucleotide. In this case, an amino acid corresponding to position 43 is preferably Gly.

Length of a partial fragment of polynucleotides encoding the polypeptides according to the present invention having reactivity with N-methyl amino acids is not particularly limited, but is for example, at least 15 consecutive bases, preferably 16 or more consecutive bases, more preferably 17 or more consecutive bases, more preferably 18 or more consecutive bases, more preferably 20 or more consecutive bases, more preferably 25 or more consecutive bases, more preferably 28 or more consecutive bases, more preferably 30 or more consecutive bases, more preferably 32 or more consecutive bases, more preferably 35 or more consecutive bases, more preferably 40 or more consecutive bases, and more preferably 50 or more consecutive bases.

In addition to the partial fragment of polynucleotides encoding ARSs according to the present invention as described above, oligonucleotides according to the present invention may also further comprise (an) oligonucleotide(s) consisting of other sequences at its/their both or either end(s) (5′ and/or 3′ end). An oligonucleotide according to the present invention is, for example, 500 bases or less in length, more preferably 300 bases or less in length, more preferably 200 bases or less in length, more preferably 100 bases or less in length, more preferably 70 bases or less in length, more preferably 60 bases or less in length, and more preferably 50 bases or less in length.

Oligonucleotides according to the present invention are useful for producing nucleic acids encoding the polypeptides according to the present invention (e.g., useful for introducing mutations), and also useful for detecting nucleic acids encoding the polypeptides according to the present invention. For example, an oligonucleotide according to the present invention can also be used as a probe in DNA hybridization or RNA hybridization operations. An example of a DNA for the purpose of using as a probe include a partial base sequence of 20 or more consecutive bases hybridizing with a polynucleotide according to the present invention, preferably, a partial base sequence of 30 or more consecutive bases, more preferably a partial base sequence of 40 or 50 or more consecutive bases, more preferably a partial base sequence of 100 or more consecutive bases, more preferably a partial base sequence of 200 or more consecutive bases, and particularly preferably a partial base sequence of 300 or more consecutive bases.

Polypeptides, polynucleotides, and oligonucleotides according to the present invention can be included in compositions in combination with carriers or vehicles, appropriately. The compositions can be produced using any method known to those skilled in the art. The polypeptides, polynucleotides, and oligonucleotides according to the present invention can be appropriately combined with, for example, pharmacologically acceptable carriers or vehicles, specifically, sterile water, physiological saline, vegetable oil, emulsifying agents, suspending agents, surfactants, stabilizers, flavoring agents, excipients, vehicles, preservatives, bonding agents, or the like. These can be mixed together to be formulated in unit dosage form required by generally-accepted pharmaceutical practices.

The present invention also provides cells transformed with a polynucleotide (including DNA and RNA) encoding a mutant N-methyl aminoacyl-tRNA synthetase as described above. Such cells may be prokaryotic cells or eukaryotic cells.

When a mutant N-methyl aminoacyl-tRNA synthetase according to the present invention expressed in cells is directly used for protein synthesis in the cells, cells depending on the intended use can be used. Any known method can be adopted for the transformation.

The present invention also provides a method for producing polypeptide containing N-methyl amino acids using the ARS with an altered amino acid sequence according to the present invention as described above.

The ARSs with altered amino acid sequences according to the present invention are used not only in cells but also in vitro (in a cell-free system).

In either case, unlike prior art using chemical synthesis such as the pdCpA method, tRNAs can be acylated repeatedly during a translation reaction, and therefore N-methyl aminoacyl-tRNAs can be supplied continuously, and addition of a large amount of tRNAs, which may inhibit translation, can be avoided.

Accordingly, the present invention provides a method for producing non-natural amino acids efficiently, selectively, in particular regioselectively, and in large amounts.

### General Method for Preparing a Modified ARS According to the Present Invention

A modified ARS according to the present invention can be produced using any known genetic recombination technique as mentioned above, and generally can be prepared as follows: first, site-directed mutation is introduced into a specific position or a specific amino acid by PCR using a plasmid comprising the natural ARS gene as a template with appropriate primers. The template plasmid is digested with a restriction enzyme, and then E. coli or the like is transformed with the digested plasmid. The plasmid into which the desired mutation has been introduced is cloned.

If a second amino acid mutation is introduced, subsequently the introduction of site-directed mutation using the plasmid having the mutation introduced into the specific position as a template with appropriate primers is repeated as with the description above, which results in the construction of a plasmid DNA encoding the polypeptide in which the two amino acids are substituted. A similar procedure can be followed when introducing further amino acid mutations.

E. coli BL21 strain or the like is co-transformed with the constructed DNA and the plasmid pREP4 encoding lac repressor (LacI) or the like, and the obtained transformed strain is isolated and cultured followed by expression induction with IPTG. The obtained strain is then disrupted, and the supernatant is passed through an affinity column for His-tag to purify the mutant ARS.

Alternatively, the following method can be used to prepare a mutant ARS. Specifically, a sequence of a certain mutant ARS is genetically synthesized and inserted into an expression vector, and proteins are allowed to be expressed. Affinity columns for different purification tags are used to purify the protein of interest.

Methods for producing a mutant N-methyl aminoacyl-tRNA synthetase according to the present invention, which is modified by substituting an amino acid at a specific position with another amino acid, are not limited to the methods as described above, and various genetic engineering techniques including known point mutation techniques and gene synthesis techniques, methods for introducing modified fragments using restriction enzymes, and the like may be used. Also, expression is not limited to expression in E. coli, and animal cells and cell-free translation systems may also be used. Purification methods are also not limited to methods in an affinity column for polyhistidine, and various peptide-tags and purification columns may be used.

### Method for Confirming Substrate Specificity of the Obtained Modified ARS According to the Present Invention

Substrate specificity of the obtained modified products can be confirmed by any of the following three assay methods, for example.

The first method can confirm by mass spectroscopy that N-methyl amino acids corresponding to codons on mRNA are introduced into a peptide by performing a translation reaction in a cell-free translation system reconstituted with the modified ARS rather than wild-type ARS and further N-methylated amino acids rather than natural amino acids, and performing aminoacylation with N-methyl amino acids in the translation system.

In the second method, peptides to be produced are labeled in a translation experiment using amino acids labeled with radioactive isotopes or fluorescent molecules, and the peptides are separated and visualized by electrophoresis or an analytical column to estimate yield of the peptides. In this method, translational introduction efficiency of corresponding N-methyl amino acids provided by using the modified ARS is increased compared to that provided by using wild-type ARS, and therefore the radioactivity or fluorescence, which is observed by electrophoresis or chromatogram, of the peptide produced using the modified ARS is observed more strongly than that of the peptide produced using wild-type ARS.

In the third method, three molecules, a modified ARS, a tRNA and an N-methyl amino acid corresponding to the modified ARS are reacted together in vitro, and the resulting N-methyl aminoacyl-tRNA is separated from unreacted tRNA by electrophoresis to quantify efficiency of the acylation reaction. The acylated tRNA produced using the modified ARS is detected more than that produced using wild-type ARS.

As mentioned above, the modified ARSs according to the present invention have increased reactivity with N-methyl amino acids compared to unmodified ARSs. The increased reactivity with N-methyl amino acids may be an increase in reaction rate, an increase in the amount of reaction products acylated with N-methyl amino acids, or an increase in substrate specificity to N-methyl amino acids. The increased reactivity with N-methyl amino acids may also be qualitative or quantitative. For example, the reactivity with N-methyl amino acids is considered to have increased in a reaction performed under the same conditions except for the use of an unmodified ARS or a modified ARS when the reactivity (such as reaction rate or the amount of reaction products) of the modified ARS to the substrate N-methyl amino acid is significantly increased compared to the reactivity of the unmodified ARS to N-methyl amino acids. Even if the reaction rate or reaction product amount in a reaction using the modified ARS and an N-methyl amino acid as substrate does not significantly increase compared to that in a reaction using the unmodified ARS, the reactivity of the modified ARS to N-methyl amino acids is considered to have increased when substrate specificity of the modified ARS to the N-methyl amino acid is increased relative to that in a reaction using unmodified amino acids. Preferably, the modified ARSs according to the present invention increase reaction rate or reaction product amount in a reaction using N-methyl amino acids as substrates compared to unmodified ARSs.

For example, the modified ARSs according to the present invention significantly increase the amount of tRNA aminoacylated with N-methyl amino acids or the amount of peptides incorporating N-methyl amino acids, as measured under the same conditions except for using either unmodified ARS or modified ARS, preferably by at least 10%, preferably by 20%, preferably by 1.3 times or more, preferably by 1.5 times or more, preferably by 2 times or more, preferably by 3 times or more, further preferably by 5 times or more, further preferably by 10 times or more, further preferably by 20 times or more, further preferably by 30 times or more, further preferably by 50 times or more, and further preferably by 100 times or more. Alternatively, the modified ARSs according to the present invention significantly increase the amount ratio of “products of the reaction with N-methyl amino acids/products of the reaction with unmodified amino acids”, as measured under the same conditions except for using either unmodified ARS or modified ARS, preferably by at least 10%, preferably by 20%, preferably by 1.3 times or more, preferably by 1.5 times or more, preferably by 2 times or more, preferably by 3 times or more, further preferably by 5 times or more, further preferably by 10 times or more, further preferably by 20 times or more, further preferably by 30 times or more, further preferably by 50 times or more, further preferably by 100 times or more compared to unmodified ARSs. Alternatively, when a nucleic acid encoding a polypeptide containing consecutive N-methyl amino acids is allowed to be translated, the modified ARSs according to the present invention significantly increase the production amount of the polypeptide containing consecutive N-methyl amino acids, as measured under the same conditions except for using either unmodified ARS or modified ARS, preferably by at least 10%, preferably by 20%, preferably by 1.3 times or more, preferably by 1.5 times or more, preferably by 2 times or more, preferably by 3 times or more compared to unmodified ARSs. The consecutive N-methyl amino acids may be, for example, two and/or three consecutive N-methyl amino acids.

The reactivity with N-methyl amino acids can be determined according to a confirmation method as described above. Specifically, for example, ribosomally-produced peptides are electrophoresed, and band intensity of peptides that have incorporated N-methyl amino acids and peptides that have not incorporated N-methyl amino acids can be measured qualitatively or quantitatively to determine the reactivity. Alternatively, mass spectral peaks can be measured, and peaks of peptides that have incorporated N-methyl amino acids and peaks of peptides that have not incorporated N-methyl amino acids can be measured to determine the reactivity.

For example, substrate specificity of a modified ARSs according to the present invention is significantly increased, preferably by at least 10%, preferably by 20%, preferably by 1.3 times or more, preferably by 1.5 times or more, preferably by 2 times or more, preferably by 3 times or more, further preferably by 5 times or more, further preferably by 10 times or more, and further preferably by 20 times or more compared to unmodified ARSs when peptides are synthesized in the presence of unmodified amino acids or N-methyl amino acids and the amount ratio of “products of the reaction with N-methyl amino acids/products of the reaction with unmodified amino acids” is measured.

The reaction conditions may be appropriately determined as long as the conditions used for unmodified ARSs and for modified ARSs are the same. The substrate concentration of unmodified amino acids and N-methyl amino acids at the reaction may be appropriately adjusted, and may be any concentration condition as long as reactivity with N-methyl amino acids is increased. Preferably, unmodified amino acids may not be added (may be only endogenous amino acids originally contained in a cell-free translation system), or may be appropriately adjusted in the range from 0.1 μM to 1 mM, for example, 0.1 μM to 500 μM, 0.1 μM to 250 μM, 0.1 μM to 100 μM, or 0.1 μM to 50 μM for reactions. N-methyl amino acids may be appropriately adjusted in the range from, for example, 50 μM to 10 mM, for example, 100 μM to 5 mM, 200 μM to 2 mM, or 500 μM to 1 mM for reactions.

Modified ARSs obtained by the preparation method as described above can be used to produce peptides and peptide-mRNA fusions that have incorporated N-methyl amino acids in a site-specific manner.

Moreover, ARSs used herein are highly conserved among biological species. Therefore, it is clear that the method according to the present invention can be generally applied to modification of N-methyl amino acid-tRNA synthetases from other biological species.

The modified ARS according to the present invention can be used to produce peptides having a particular amino acid substituted with its non-natural, N-methylated amino acid, in a prokaryotic translation. Such peptides can be produced using ARSs that are derived from other organisms and modified in a similar way as the present invention.

Acylation Reaction of tRNAs Using the Modified ARSs According to the Present Invention

Use of the modified ARSs according to the present invention makes it possible to employ N-methyl amino acids as substrates in acylation of tRNAs. The modified ARSs according to the present invention can acylate tRNAs with N-methyl amino acids and to ribosomally produce N-methyl amino acid-containing peptides.

In order to produce N-methyl aminoacyl-tRNAs using the modified ARSs according to the present invention, the modified ARSs, N-methyl amino acids corresponding to the modified ARSs, and tRNAs may be reacted in vitro as in chemical synthesis such as the pdCpA method. The products, N-methyl aminoacyl-tRNAs may be isolated using any known nucleic acid purification technique such as ethanol precipitation, and the isolated product may be added to a translation system. This leads to production of polypeptides or polypeptide-mRNA fusions that have introduced N-methyl amino acids at the intended positions.

Unlike chemical synthesis which requires reactions performed in a reaction solution comprising only a particular tRNA and the N-methyl amino acid corresponding to the tRNA as substrates, three molecules, a modified ARS, N-methyl amino acid, and tRNA are used to precisely and efficiently produce the intended N-methyl aminoacyl-tRNA even in a mixture of translation reaction solution comprising other tRNAs and other amino acids because the modified ARSs according to the present invention have high substrate specificity for tRNAs and amino acids. Accordingly, the isolation and purification steps as described above are not essential. A reaction solution in which tRNAs have been acylated with N-methyl amino acids can be directly used in a translation reaction, or a translation reaction can be performed at the same time as an acylation reaction of tRNAs with N-methyl amino acids. The modified ARSs according to the present invention are very convenient because the reactions using the modified ARSs require no chemical synthesis of substrates such as pdCpA amino acids and activated amino acids and can be performed using commercially available reagents.

The most important characteristics include no requirement of stoichiometric consideration for a tRNA of interest in performing peptide translation (peptide expression) using a modified ARS according to the present invention. In chemical synthesis in which aminoacyl-tRNAs are supplied, N-methyl aminoacyl-tRNAs are consumed during the reaction, decreasing the reaction efficiency. However, if ARSs are used, translational efficiency is good even in peptide synthesis in which multiple N-methyl amino acids are introduced. Aminoacyl-tRNAs are constantly deacylated in hydrolytic reactions or transpeptidation reactions in translation systems, but the modified ARSs according to the present invention recognize released tRNAs and newly acylate the tRNAs with N-α amino acids, which results in a constant supply of N-α aminoacyl-tRNAs in the translation system. Therefore, the amount of tRNA required is less than the amount of produced polypeptide. Addition of large amounts of tRNA itself contributes to reduction of peptide yield. Therefore, the modified ARSs according to the present invention are useful for producing N-α amino acid-containing peptides at a high translational efficiency and producing highly diverse peptide libraries.

Moreover, because the modified ARSs according to the present invention have increased reactivity with N-methyl amino acids compared to natural ARSs, the modified ARSs can acylate tRNAs in the presence of N-methyl amino acids at a concentration lower than a concentration of N-methyl amino acids used for natural ARSs. In other words, the absolute amount of N-methyl amino acids required for peptide expression using the modified ARSs is not as much as that of N-methyl amino acids required for peptide expression using natural ARSs.

In addition, the modified ARSs according to the present invention can be used to translate N-methyl amino acid-containing peptides at a concentration lower than a concentration of natural ARSs used for translating N-methyl amino acid-containing peptides. In other words, the absolute amount of the modified ARS according to the present invention required for peptide expression is significantly less than in translation of N-methyl amino acid-containing peptides using natural ARSs.

These characteristics are important for improving orthogonality of aminoacylation reaction. Namely, in order to translate N-methyl amino acid-containing peptides, the more the substrate N-methyl amino acid, or an ARS corresponding to the N-methyl amino acid, is needed at a concentration higher than other natural amino acids, or ARSs corresponding to the other natural amino acids, the more it makes it easier for non-specific reactions to happen, such non-specific reactions being: ARS that should be normally using an N-methyl amino acid as a substrate may use other natural amino acids for acylation reaction; or an N-methyl amino acid that is present in excess may be used in acylation reactions by ARSs corresponding to other natural amino acids. This results in the obtained translated peptides being a mixture of N-methyl amino acid-containing peptides and peptides containing corresponding natural amino acid, which may cause issues for peptide libraries. However, it is expected that this issue can be minimized by using the modified ARSs according to the present invention.

Accordingly, the characteristics of the modified ARSs according to the present invention can be described as follows:
modified ARSs that catalyze acylation of tRNAs and comprise
(a) a tRNA binding site;
(b) a binding site to an N-methyl amino acid substrate; and
(c) a catalytically active site having activity to catalyze a reaction in which an acyl group is transferred from the N-methyl amino acid substrate to 3′ end of a tRNA,
wherein the modified ARSs are characterized in that they can correctly recognize tRNAs and N-methyl amino acids in a translation reaction mixture, acylates the tRNAs, and further reuse tRNAs released after the transacylation reaction to repeat acylation reactions again.

Moreover, the modified ARSs according to the present invention may comprise, in addition to (a), (b), and (c) as described above, (d) an editing site where a tRNA acylated with any undesired amino acid is hydrolyzed.

Synthesis of Acylated tRNAs Using the Modified ARSs According to the Present Invention

The modified ARSs according to the present invention can be used to synthesize tRNAs acylated with desired N-methyl amino acid substrates.

A method for producing acylated tRNAs using the modified ARSs according to the present invention, comprising the following steps:
(a) providing one or more modified ARSs according to the present invention;
(b) providing tRNAs;
(c) providing N-methyl amino acids; and
(d) contacting the modified ARSs with the tRNAs and N-methyl amino acids to acylate the tRNAs.

In addition to the steps as described above, the method may further comprise the step of (e) collecting the reaction product comprising the acylated tRNAs. The acylated tRNAs require no purification in the collecting step, and the reaction mixture can be collected and used directly. By not separating or purifying the produced aminoacyl-tRNAs from the ARSs, deacylation can be prevented.

This method uses N-methyl amino acids as substrates. Particularly preferable N-methyl amino acids include N-methylphenylalanine, N-methylvaline, N-methylthreonine, N-methyltryptophan, N-methylleucine, and/or N-methylserine. A substrate of an ARS (an amino acid corresponding to the ARS) is appropriately selected as N-methyl amino acid used as a substrate.

In a method for producing an acylated tRNA using a modified ARS according to the present invention, a tRNA that can be used is a tRNA corresponding to an ARS of a corresponding natural amino acid. The term “corresponding natural amino acid” refers to an amino acid that is not N-methylated relative to an N-methyl amino acid. For example, for N-methylphenylalanine, the corresponding amino acid is phenylalanine, and the corresponding tRNA is a tRNA recognizing the codon UUU or UUC (having the anticodon corresponding to the codon). For N-methylvaline, the corresponding amino acid is valine, and the corresponding tRNA is a tRNA recognizing the codon GUU, GUC, GUA, or GUG (having the anticodon corresponding to the codon). For N-methylserine, the corresponding amino acid is serine, and the corresponding tRNA is a tRNA recognizing the codon UCU, UCC, UCA, UCG, AGU, or AGC (having the anticodon corresponding to the codon). For N-methylthreonine, the corresponding amino acid is threonine, and the corresponding tRNA is a tRNA recognizing the codon ACU, ACC, ACA, or ACG (having the anticodon corresponding to the codon). For N-methyltryptophan, the corresponding amino acid is tryptophan, and the corresponding tRNA is a tRNA recognizing the codon UGG (having the anticodon corresponding to the codon). For N-methylleucine, the corresponding amino acid is leucine, and the corresponding tRNA is a tRNA recognizing the codon UUA, UUG, CUU, CUC, CUA, or CUG (having the anticodon corresponding to the codon). Similarly, for other N-methyl amino acids, tRNAs recognizing codons corresponding to the corresponding amino acid (having the anticodons corresponding to the codons) can be used.

When a tRNA is acylated by a modified ARS in a solution, a pellet obtained by ethanol precipitation of the reaction solution may be dissolved in a suitable buffer (such as 1 mM potassium acetate, pH 5) and added to a translation system. Typical reaction conditions include, for example, a reaction performed at 37° C. for 5 minutes to 1 hour in 0.1 M reaction buffer (pH 7.5) containing a final concentration of 0.5 μM to 40 μM of a tRNA, 0.1 μM to 10 μM of a modified ARS according to the present invention, 0.1 mM to 10 mM of an N-methyl amino acid, 0.1 mM to 10 mM ATP, and 0.1 mM to 10 mM MgCl₂.

Furthermore, for an aminoacylation reaction, a tRNA can be refolded by, for example, heating 1 to 50 μM tRNA, 10 to 200 (e.g., 50 to 200) mM HEPES-K (pH 7.0 to 8.0 (e.g., 7.6)), 1 to 100 (e.g., 10) mM KCl solution at 95° C. for 2 minutes and then left at room temperature for 5 minutes or more. This tRNA solution can be added to an acylation buffer (a final concentration of 25 to 100 (e.g., 50) mM HEPES-K [pH 7.0 to 8.0 (e.g., 7.6)], 1 to 10 (e.g., 2) mM ATP, 10 to 100 (e.g., 100) mM potassium acetate, 1 to 20 (e.g., 10) mM magnesium acetate, 0.1 to 10 (e.g., 1) mM DTT, 0.1 mg/mL Bovine Serum Albumin) to a final concentration of 1 to 40 (e.g., 10) μM, mixed with a modified ARS (a final concentration of 0.1 to 10 (e.g., 0.5) μM) and an N-methyl amino acid (a final concentration of 0.1 to 10 (e.g., 1) mM), and incubated at 37° C. for 5 to 60 (e.g., 10) minutes.

Thus, the acylation reaction using a modified ARS according to the present invention requires no activated amino acids that need substrates and need to be synthesized. The acylation reaction can be performed with commercially available N-methyl amino acids and therefore is convenient. The modified ARSs according to the present invention can be combined with their substrates to form a kit product for obtaining acylated tRNAs. The kit may at least comprise (a) one or more modified ARSs according to the present invention, (b) N-methyl amino acid(s), and (c) tRNA(s), and may further comprise a reaction buffer, a reaction vessel, instructions for use, and the like. In the kit, each of (b) N-methyl amino acid(s) and (c) tRNA(s) acts as a substrate for (a) modified ARSs. In other words, the tRNAs recognize codons corresponding to natural amino acids equivalent to the N-methyl amino acids (the tRNAs have anticodons corresponding to the codons).

### Synthesis of N-methyl Amino Acid-Containing Polypeptides Using the Modified ARSs According to the Present Invention

N-methyl amino acid-bound tRNAs can be used to produce polypeptides with N-methyl amino acids introduced into desired sites. The method comprises translating a nucleic acid encoding a polypeptide of interest in the presence of a modified ARS according to the present invention.

More specifically, for example, a method for producing an N-methyl amino acid-containing polypeptide using a modified ARS(s) according to the present invention comprises (a) providing the modified ARS(s) according to the present invention, (b) establishing a cell-free translation system reconstituted with the modified ARS(s) according to the present invention rather than wild-type ARS(s), (c) providing an mRNA having at a desired site(s) codon(s) corresponding to the anticodon(s) of a tRNA(s) that is the substrate(s) of the modified ARS(s), and (d) adding the mRNA to the cell-free translation system to produce a polypeptide with an N-methyl amino acid(s) introduced into a desired site(s). Matters particularly relevant to the production of polypeptide in (d) will be described below.

N-methyl amino acids preferably used in acylation of tRNAs with N-methyl amino acids using the modified ARSs according to the present invention include N-methylalanine, N-methylleucine, N-methyltryptophan, N-methylphenylalanine, N-methylvaline, N-methylthreonine, and/or N-methylserine, and particularly preferably N-methylphenylalanine, N-methylvaline, N-methylthreonine, N-methyltryptophan, N-methylleucine, and/or N-methylserine.

Specific methods for polypeptide synthesis may be essentially performed according to known methods, for example, performed as described in WO2013100132, and various modifications can be made. Generally, the methods can be performed according to the following description.

A suitable translation system that can be used is a cell-free translation system, typified by PURESYSTEM (Registered trademark) (BioComber, Japan), reconstituted with translation factors. In such a cell-free translation system, components of the translation system can be controlled flexibly. For example, phenylalanine and the ARS corresponding to phenylalanine are removed from the translation system and instead, N-methylphenylalanine and the modified phenylalanine-ARS according to the present invention can be added. This achieves introduction of N-methylphenylalanine into a codon, such as UUU and UUC, encoding phenylalanine in a site-specific manner.

In a cell-free translation system, ribonucleosides preferably used are ATP and GTP at 0.1 mM-10 mM. A buffer preferably used is HEPES-KOH at 5 mM-500 mM and pH 6.5-8.5. Examples of other buffers include, but are not limited to, Tris-HCl, phosphate, and the like. Salts that can be used are acetates such as potassium acetate and ammonium acetate, and glutamates such as potassium glutamate, which are preferably used at 10 mM-1000 mM. A magnesium component preferably used is magnesium acetate at 2 mM-200 mM. Examples of other magnesium components include, but are not limited to, magnesium chloride and the like. Components in an energy-regenerating system preferably used are creatine kinase at 0.4 μg/mL-40 μg/mL and creatine phosphate at 2 mM-200 mM. Other energy-regenerating systems, typified by pyruvate kinase and phosphoenol pyruvate, may also be used. A nucleoside converting enzyme preferably used is myokinase at 0.1 unit/mL-10 unit/mL or nucleoside diphosphate kinase at 0.2 μg/mL-20 μg/mL. A diphosphatase preferably used is inorganic pyrophosphatase at 0.2 unit/mL-20 unit/mL. A polyamine preferably used is spermidine at 0.2 mM-20 mM. Examples of other polyamines include, but are not limited to, spermine and the like. A reducing agent preferably used is dithiothreitol at 0.1 mM-10 mM. Examples of other reducing agents include, but are not limited to, β-mercaptoethanol and the like. A tRNA preferably used is, for example, E. coli MRE600 (RNase-negative)-derived tRNA (Roche) at 0.5 mg/mL-50 mg/mL. Other tRNAs from E. coli may also be used. A formyl donor and an enzyme preferably used to synthesize formylmethionine used in a translational initiation reaction are 10-HCO—H4 folate at 0.1 mM-10 mM and methionyl-tRNA transformylase at 0.05 μM-5 μM. A translation initiation factor preferably used is IF1 at 0.5 μM-50 μM, IF2 at 0.1 μM-50 μM, or IF3 at 0.1 μM-50 μM. A translation elongation factor preferably used is EF-G at 0.1 μM-50 μM, EF-Tu at 1 μM-200 μM, or EF-Ts at 1 μM-200 μM. A translation termination factor preferably used is RF-2, RF3, or RRF at 0.1 μM-10 μM. Ribosome is preferably used at 1 μM-100 μM. There are 20 types of aminoacyl-tRNA synthetases, but only the enzymes corresponding to amino acids included in a peptide to be synthesized may be added. For example, ArgRS, AspRS, LysRS, MetRS, and TyrRS are all preferably used at 0.01 μM-1 μM. Amino acids used as substrates for peptide synthesis are natural 20 amino acids, which compose proteins, and derivatives thereof. Only amino acids included in a peptide to be synthesized are preferably used at 0.25 mM-10 mM. An mRNA as a template for peptide synthesis is preferably used at 0.1 μM-10 μM. When an mRNA is transcribed from a template DNA in a cell-free translation system, commercially available enzymes such as T7 RNA polymerase, T3 RNA polymerase, and SP6 RNA polymerase can be used, and the enzymes suitable for a promoter sequence in the template DNA may be appropriately selected and are preferably used at 1 μg/mL-100 μg/mL. In this case, nucleosides CTP and UTP, which are substrates, are preferably used at 0.1 mM-10 mM. A solution containing mixed these components can be left, for example, at 37° C. for 1 hour to achieve translational synthesis of peptides. Temperature and reaction time are not limited to those.

The modified ARSs according to the present invention can also be used in combination with other techniques for introducing non-natural amino acids, such as the pdCpA method or the Flexizyme method. For example, polypeptides containing both N-methylglycine and N-methylphenylalanine can be synthesized when the aminoacyl-tRNA to which N-methylglycine is attached by the pdCpA method is added to a translation system at the same time as addition of N-methylphenylalanine and the modified phenylalanine ARS.

Alternatively, polypeptides containing N-methyl amino acids can be expressed in cells by inserting the modified ARSs according to the present invention into an expression vector or genome, expressing the modified ARSs in cells, and using N-methyl amino acids added to a medium as substrates.

tRNAs that can be used include a tRNA for a natural amino acid corresponding to an N-methyl amino acid. The tRNAs may be tRNAs that contain modified bases and are purified from a living body, or tRNAs that do not contain modified bases and are produced using an in vitro transcription reaction. Mutant tRNAs, which have a mutation in a portion of tRNA other than the portion recognized by an ARS, can also be used as substrates.

### EXAMPLES

The content of the present invention as mentioned above will be described in more detail in the Examples below, but the Examples are illustrative and are not intended to limit the scope of the present invention. Various variations and modifications that can be made by those skilled in the art based on the description in the specification and claims are also included in the present invention.

All prior art literatures cited here are incorporated herein by reference.

### Example 1: N-methylphenylalanine-accepting ARS

### Preparation of Plasmids for Wild-Type and Mutant PheRSs

Using a plasmid (pQE-32(2) 2_wtPheRS) comprising the ORF sequence of wild-type PheRS α subunit gene of E. coli (SEQ ID NOs: 27, 28) as starting material, mutant PheRS plasmids (having His-tag (6×His) at the N-terminus) listed in Table 1 were constructed by introducing site-directed mutations using PCR. Specifically, 2 μL of 10 ng/μL template, 10 μL of 2×KOD Fx buffer (TOYOBO, KFX-101), 0.6 μL of 10 μM forward primer, 0.6 μL of 10 μM reverse primer, 4 μL of 2 mM dNTP, 0.4 μL of KOD FX (TOYOBO, KFX-101), and 2.4 μL of H₂O were mixed together. Thereafter, the resulting reaction solution was heated at 94° C. for 2 minutes and then subjected to 10 cycles, each consisting of heating at 98° C. for 10 seconds and heating at 68° C. for 7 minutes, to amplify the mutant gene. The combinations of the template plasmid, forward primer, and reverse primer used are listed in Table 2. Each sequence of primers “F.F02” through “F.F05” corresponds to SEQ ID NOs: 30-33 in ascending order, and each sequence of primers “R.F02” through “R.F05” corresponds to SEQ ID NOs: 34-37 in ascending order. 0.5 μL of 10 U/μL DpnI was then added to the PCR reaction solution and further incubated at 37° C. for 1.5 hours to digest the template DNA, and the resulting mutant DNA was purified. E. coli XL-1 Blue strain (STRATAGENE, 200236) was then co-transformed with the resulting mutant gene DNA and pREP4 (Invitrogen, V004-50) encoding the lacI gene. The transformants were seeded onto agar containing ampicillin and kanamycin. The plasmids of interest were purified from the resulting clones. The mutations were confirmed to be introduced into the plasmids.

TABLE 1

| Name | position 169 | position 171 |
|---|---|---|
| PheRS01(wt) | Q | S |
| PheRS02 | Q | A |
| PheRS03 | Q | G |
| PheRS04 | A | S |
| PheRS05 | G | S |

TABLE 2

| Name | Template | 5′ primer | 3′ primer |
|---|---|---|---|
| PheRS02 | PheRS01(wt) | F. F02 | R. F02 |
| PheRS03 | PheRS01(wt) | F. F03 | R. F03 |
| PheRS04 | PheRS01(wt) | F. F04 | R. F04 |
| PheRS05 | PheRS01(wt) | F. F05 | R. F05 |

### Small Scale Expression of Wild-Type and Mutant PheRSs

Next, a plasmid comprising a resulting mutant gene and the gene encoding PheRS β subunit was introduced into E. coli, and a heterodimer comprising the mutant protein was expressed. First, E. coli BL21 strain transformed with the mutant plasmid and pREP4 (Invitrogen, V004-50) was cultured at 37° C. in 4 mL of LB medium containing kanamycin, ampicillin, and 0.5% glucose. Subsequently, when the OD value at 600 nm reached 0.4 to 0.8, IPTG was added to a final concentration of 0.5 mM. After further culturing at 37° C. for 4 hours, the bacterial pellets were collected using a centrifuge.

### Small Scale Purification of Wild-Type and Mutant PheRSs

Next, the resulting bacterial pellets were disrupted, and the mutant protein of interest was purified from the supernatant. Specifically, the bacterial pellets as described above were suspended in 600 μL of CHAPS solution (0.5% CHAPS (DOJINDO: 349-04722), 50% TBS (TaKaRa, T903)) and mixed with 6 μL of 30 U/μl rLysozyme (Novagen, 71110-3), and 2 μL of 2.5 U/μL benzonase nuclease (Novagen, 70746-3) followed by incubation at room temperature for 30 minutes. Imidazole was then added to a final concentration of 15 mM, and an insoluble fraction was separated by centrifugation. Then, the mutant protein was purified from the resulting supernatant using QIAGEN Ni-NTA spin column kit (Qiagen, 31314) according to the product manual. Finally, excess imidazole was removed using a desalting column, PD miniTrap G-25 (GE Healthcare, 28-9180-07) according to the product manual.

### Large Scale Purification of Wild-Type and Mutant PheRSs

The mutant protein confirmed to have activity was prepared in large scale. Specifically, E. coli BL21 strain transformed with a plasmid comprising the mutant α subunit gene and wild-type Q subunit gene and pREP4 (Invitrogen, V004-50) was cultured at 37° C. in 3 L of LB medium containing kanamycin, ampicillin, and 0.5% glucose. Then, when the OD value at 600 nm reached 0.4, IPTG was added to a final concentration of 0.5 mM. After further culturing at 37° C. for 4 hours, the bacterial pellets were collected with a centrifuge. The bacterial pellets as described above were suspended in 1 L of CHAPS solution (0.5% CHAPS (DOJINDO: 349-04722) and 50% TBS (TaKaRa, T903)), mixed with 10 μL of 30 KU/μl rLysozyme (Novagen, 71110-3), and stirred at room temperature for 10 minutes. Next, 2 mL of 1 M MgCl₂ and 320 μL of Benzonase Nuclease (Novagen, 70746-3) were added and stirred at room temperature for 20 minutes. Imidazole was then added to a final concentration of 20 mM, and an insoluble fraction was separated by centrifugation. Subsequently, the mutant protein was purified from the resulting supernatant using a column filled with 15 mL of Ni Sepharose High Performance (GE Healthcare) and AKTA10S (GE Healthcare) with an imidazole concentration gradient (initial concentration 20 mM, final concentration 500 mM). Finally, dialysis was performed three times (2 hours×2, overnight×1) using a dialysis cassette (MWCO 10,000, Slide-A-Lyzer G2 Dialysis Cassettes 70 mL, Thermo Scientific Pierce) and 3 L of stock solution (50 mM Hepes-KOH, 100 mM KCl, 10 mM MgCl₂, 1 mM DTT, pH 7.6) to obtain the mutant protein.

Aminoacylation Reaction with N-methylphenylalanine Using Wild-Type and Mutant PheRSs
Synthesis of E. coli tRNAPhe by In Vitro Transcription Reaction

E. coli tRNA (R-tRNAPhe (SEQ ID NO: 39)) was synthesized from a template DNA (D-tRNAPhe (SEQ ID NO: 38)) by in vitro transcription reaction using RiboMAX Large Scale RNA production System T7 (Promega, P1300) in the presence of 7.5 mM GMP, and purified using RNeasy Mini kit (Qiagen).

D-tRNAPhe
tRNAPhe DNA sequence:
(SEQ ID NO: 38)
GGCGTAATACGACTCACTATAGCCCGGATAGCTCAGTCGGTAGAGCAGGG
GATTGAAAATCCCCGTGTCCTTGGTTCGATTCCGAGTCCGGGCACCA
R-tRNAPhe
tRNAPhe RNA sequence:
(SEQ ID NO: 39)
GCCCGGAUAGCUCAGUCGGUAGAGCAGGGGAUUGAAAAUCCCCGUGUCCU
UGGUUCGAUUCCGAGUCCGGGCACCA

### Aminoacylation Reaction

For the aminoacylation reaction, the solution containing 40 μM transcribed tRNAPhe, 10 mM HEPES-K (pH 7.6), and 10 mM KCl solution was heated at 95° C. for 2 minutes and then left at room temperature for 5 minutes or more to refold the tRNA. This tRNA solution was added to a final concentration of 10 μM to an acylation buffer (in final concentrations of 50 mM HEPES-K [pH 7.6], 2 mM ATP, 100 mM potassium acetate, 10 mM magnesium acetate, 1 mM DTT, 2 mM spermidine, 0.1 mg/mL Bovine Serum Albumin), mixed with wild-type or mutant PheRS (final concentration 0.5 μM) and phenylalanine (final concentration 0.25 mM, Watanabe Chemical Industries, Ltd., G00029) or N-methylphenylalanine (final concentration 1 mM, Watanabe Chemical Industries, Ltd., J00040), and incubated at 37° C. for 10 minutes. Four volumes of a loading buffer (90 mM sodium acetate [pH 5.2], 10 mM EDTA, 95% (w/w) formamide, 0.001% (w/v) xylene cyanol) was added to the reaction solution and analyzed with acidic PAGE (12% (w/v) polyacrylamide gel, pH 5.2) containing 6 M urea, and aminoacylation activity was detected by separating unreacted tRNA and aminoacylated tRNA. RNA was stained with SYBR Gold (Life Technologies) and detected with LAS4000 (GE Healthcare).

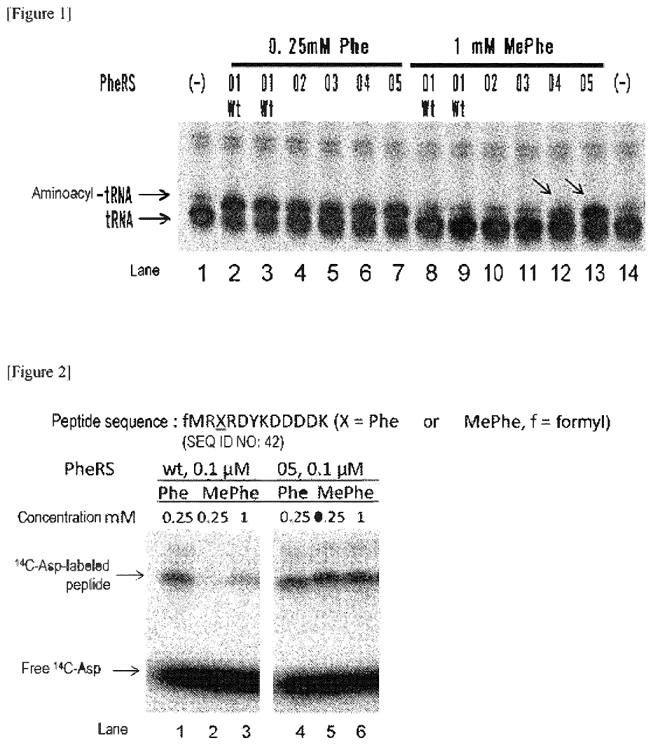

The activity for the aminoacylation reaction was assessed, and mutants 04 and 05 had increased activity for aminoacylation with N-methyl-phenylalanine compared to the wildtype (FIG. 1). The base sequence of mutant 04 is set forth in SEQ ID NO: 12, and the amino acid sequence of mutant 04 is set forth in SEQ ID NO: 1. The base sequence of mutant 05 is set forth in SEQ ID NO: 13, and the amino acid sequence of mutant 05 is set forth in SEQ ID NO: 2.

### Translational Introduction of N-methylphenylalanine Using Wild-Type and Mutant PheRSs

### Synthesis of Template DNA-F by In Vitro Transcription Reaction

A template mRNA for translation (R-F (SEQ ID NO: 41)) was synthesized from a template DNA (D-F (SEQ ID NO: 40)) by in vitro transcription reaction using RiboMAX Large Scale RNA production System T7 (Promega, P1300) and purified using RNeasy Mini kit (Qiagen).

D-F
DNA sequence:
(SEQ ID NO: 40)
GGCGTAATACGACTCACTATAGGGTTAACTTTAACAAGGAGAAAAACATG
CGTTTCCGTGACTACAAGGACGACGACGACAAGTAAGCTTCG
R-F
RNA sequence:
(SEQ ID NO: 41)
GGGUUAACUUUAACAAGGAGAAAAACAUGCGUUUCCGUGACUACAAGGAC
GACGACGACAAGUAAGCUUCG

### Cell-Free Translation System

In order to confirm translational introduction of N-methylphenylalanine, a desired polypeptide containing N-methylphenylalanine was ribosomally synthesized by adding an N-methyl amino acid and PheRS to a cell-free translation system. The translation system used was PURE system, a reconstituted cell-free protein synthesis system from E. coli. Specifically, wild-type or mutant PheRS and phenylalanine or N-methylphenylalanine were added to a solution containing a basic cell-free translation solution (1 mM GTP, 1 mM ATP, 20 mM creatine phosphate, 50 mM HEPES-KOH pH 7.6, 100 mM potassium acetate, 9 mM magnesium acetate, 2 mM spermidine, 1 mM dithiothreitol, 0.5 mg/ml E. coli MRE600 (RNase negative)-derived tRNA (Roche), 4 μg/ml creatine kinase, 3 μg/ml myokinase, 2 unit/ml inorganic pyrophosphatase, 1.1 μg/ml nucleoside diphosphate kinase, 0.6 μM methionyl-tRNA transformylase, 0.26 μM EF-G, 0.24 μM RF2, 0.17 μM RF3, 0.5 μM RRF, 2.7 μM IF1, 0.4 μM IF2, 1.5 μM IF3, 40 μM EF-Tu, 44 μM EF-Ts, 1.2 μM ribosome, 0.03 μM ArgRS, 0.13 μM AspRS, 0.11 μM LysRS, 0.03 μM MetRS, 0.02 μM TyrRS (wherein the proteins prepared by the inventors were essentially prepared as His-tagged proteins)), and 1 μM template mRNA, each 250 μM arginine, aspartic acid, lysine, methionine, and tyrosine, and left at 37° C. for 1 hour to ribosomally synthesize the peptide.

### Detection by Electrophoresis

A peptide expression experiment was performed by using aspartic acid labeled with a radioisotope for detecting peptides into which N-methylphenylalanine is ribosomally introduced. Specifically, a solution containing 1 μM template mRNA (R-F (SEQ ID NO: 41)), arginine, lysine, methionine, and tyrosine (each final concentration 250 μM), and ¹⁴C-aspartic acid (final concentration 37 μM, Moravek Biochemicals, MC139) added to the cell-free translation system as described above was prepared. Wild-type PheRS or the mutant PheRS 05 (final concentration 0.1 μM) and phenylalanine (final concentration 250 μM) or N-methylphenylalanine (final concentration 1 mM or 250 μM) were added to the solution and incubated at 37° C. for 60 minutes. An equal volume of 2× sample buffer (TEFCO, cat No. 06-323) was added to the resulting translation reaction solution and heated at 95° C. for 3 minutes followed by electrophoresis (16% Peptide-PAGE mini, TEFCO, TB-162). After electrophoresis, the gel was dried using Clear Dry Quick Dry Starter KIT (TEFCO, 03-278), exposed to an imaging plate (GE Healthcare, 28-9564-75) for about 16 hours, detected using a Bioanalyzer System (Typhoon FLA 7000, GE Healthcare), and analyzed with ImageQuantTL (GE Healthcare).

Almost no bands of peptides that were ribosomally synthesized were observed in the presence of 0.1 μM wild-type PheRS when 0.25 mM or 1 mM N-methylphenylalanine was added (FIG. 2). Meanwhile, peptide bands were observed in the presence of 0.1 μM mutant PheRS 05 even when 0.25 mM N-methylphenylalanine was added. The results can provide confirmation that the mutant PheRS 05 had increased aminoacylation activity with N-methylphenylalanine and that peptides containing N-methylphenylalanine were ribosomally synthesized at a high yield.

### Detection by Mass Spectroscopy

Mass spectroscopy was performed using MALDI-TOF MS for detecting peptides into which N-methylphenylalanine is ribosomally introduced. Specifically, a solution containing 1 μM template mRNA (R-F (SEQ ID NO: 41)) and amino acids, arginine, lysine, methionine, tyrosine, and aspartic acid (each final concentration 250 μM) added to the cell-free translation system as described above was prepared. PheRS (final concentration 0.1 μM) and phenylalanine (final concentration 250 μM) or N-methylphenylalanine (final concentration 1 mM or 250 μM) were added to the solution and incubated at 37° C. for 60 minutes. The resulting translation reaction products were purified with SPE C-TIP (Nikkyo Technos Co., Ltd) and analyzed with MALDI-TOF MS. α-Cyano-4-hydroxycinnamic acid was used as a matrix for translation products.

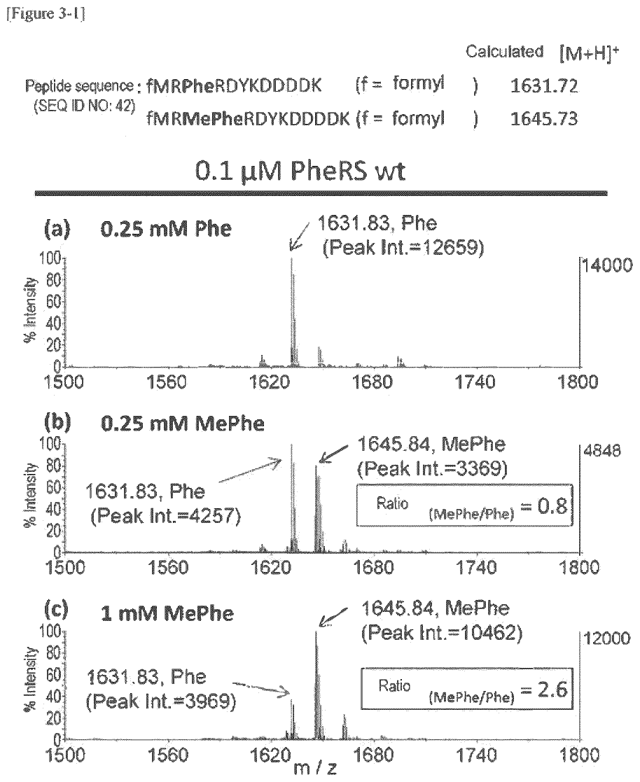

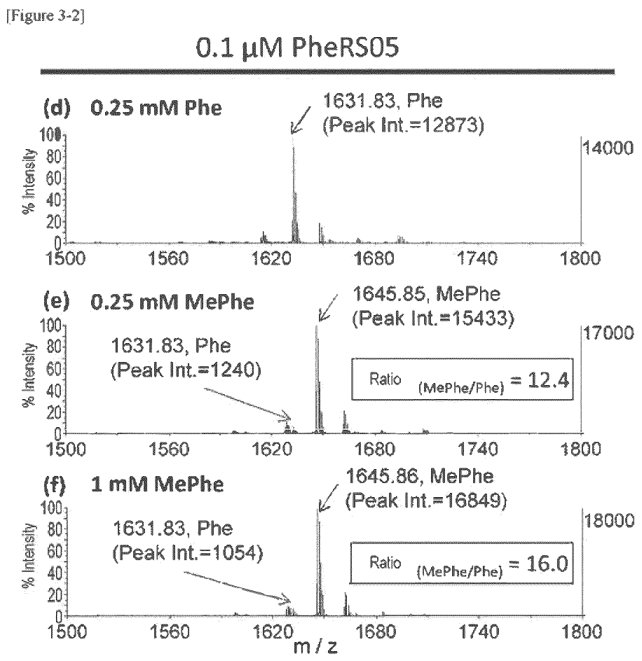

When a translation experiment was performed as a control experiment in the presence of 0.1 μM wild-type PheRS (SEQ ID NO: 28) or mutant PheRS 05 (SEQ ID NO: 2) with 250 μM phenylalanine added, the peak of the peptide into which phenylalanine was introduced (Calculated [M+H]+=1631.7) was detected in both experiments using wild-type PheRS and the mutant PheRS 05 (FIG. 3(a), (d)).

Translational synthesis was then performed by using wild-type PheRS and adding 0.25 mM N-methylphenylalanine, and the peaks of the peptide containing N-methylphenylalanine (Calculated [M+H]+=1645.7) and the peptide containing phenylalanine were observed (FIG. 3 (b)). This phenomenon is thought to be due to wild-type PheRS recognizing phenylalanine contaminated in N-methylphenylalanine, the subsequent occurrence of the aminoacylation reaction, and proceeding of translational synthesis. Furthermore, the peak intensity of the peptide containing N-methylphenylalanine was increased under the condition of 1 mM N-methylphenylalanine (FIG. 3(c)). The ratio of peak intensities at each amino acid concentration was calculated (the calculating formula: the ratio of peak intensity=the peak intensity of the peptide containing N-methylphenylalanine/the peak intensity of the peptide containing phenylalanine) to be 0.8 at 0.25 mM and 2.6 at 1 mM.

Next, a translational synthesis was performed by using the mutant PheRS 05 and adding 0.25 mM or 1 mM N-methylphenylalanine, and the peak of the peptide containing N-methylphenylalanine was strongly detected, and the ratio of peak intensity of the peptide containing N-methylphenylalanine to the peak intensity of the peptide containing phenylalanine was 12.4 at 0.25 mM and 16.0 at 1 mM (FIG. 3 (e), (f)). These results confirmed that the mutant PheRS 05 had increased aminoacylation activity with N-methylphenylalanine compared to wild-type PheRS, and consequently, translational synthesis of the peptide containing N-methylphenylalanine was promoted.

Peptide sequence P-F1
(SEQ ID NO: 42)
formylMetArgPheArgAspTyrLysAspAspAspAspLys
Peptide sequence P-MeF1
(SEQ ID NO: 42)
formylMetArg[MePhe]ArgAspTyrLysAspAspAspAspLys

MALDI-TOF MS:

Calc. m/z: [H+M]+=1631.7 (the peptide corresponding to the sequence P-F1)

Calc. m/z: [H+M]+=1645.7 (the peptide corresponding to the sequence P-MeF1)

Comparison with the pdCpA Method

Incorporation efficiency of N-methylphenylalanine using an ARS according to the present invention was compared with that in the pdCpA method. Translation was performed using a sequence containing one, two consecutive, or three consecutive phenylalanines in a cell-free translation system, and the synthesized peptide band was detected by electrophoresis. The amount of the peptide synthesized using a mutant PheRS according to the present invention (PhrRS05 (SEQ ID NO: 2)) was compared with the amount of the peptide synthesized using the pdCpA method. It is confirmed that the amount of the peptide synthesized using the mutant PheRS according to the present invention was detected more than the amount of the peptide synthesized using the pdCpA method in all translations and that translational efficiency obtained by using the mutant PheRS was also higher than that obtained by using the pdCpA method. Particularly, it is revealed that the amount of the peptide produced by translating sequences containing two consecutive and three consecutive N-methylphenylalanines using the mutant PheRS according to the present invention (PhrRS05) was 4 to 8 times higher than the amount of the peptide produced using the pdCpA method.

### Example 2: N-methylvaline-accepting ARS

### Preparation of Plasmids for Wild-Type and Mutant ValRSs

The mutant ValRS plasmids (having His-tag (6×His) at the N-terminus) listed in Table 3 were constructed by introducing site-directed mutations using PCR into the plasmid (PQE-32(2) 2_wtVALRS) comprising the ORF sequence of wild-type ValRS gene of E. coli (SEQ ID NOs: 23, 24), which was used as starting material. Specifically, 2 μL of 10 ng/μL template, 10 μL of 2×KOD Fx buffer (TOYOBO, KFX-101), 0.6 μL of 10 μM forward primer, 0.6 μL of 10 μM reverse primer, 4 μL of 2 mM dNTP, 0.4 μL of KOD FX (TOYOBO, KFX-101), and 2.4 μL of H₂O were mixed together. Then, the resulting reaction solution was heated at 94° C. for 2 minutes and then subjected to 10 cycles, each consisting of heating at 98° C. for 10 seconds and heating at 68° C. for 7 minutes, to amplify the mutant gene. The combinations of the template plasmid, forward primer, and reverse primer used are listed in Table 4. Each sequence of primers “F.V2” through “F.V19”, “F.V46” through “F.V48”, and “F.V13-01” through “F.V13-16” corresponds to SEQ ID NOs: 43-79 in ascending order. Each sequence of primers “R.V2” through “R.V19”, “R.V46” through R.V48”, and “R.V13-01” through “R.V13-16” corresponds to SEQ ID NOs: 80-116 in ascending order. 0.5 μL of 10 U/μL DpnI was then added to the PCR reaction solution and further incubated at 37° C. for 1.5 hours to digest the template DNA, and the resulting mutant DNA was purified. E. coli XL-1 Blue strain (STRATAGENE, 200236) was then co-transformed with the resulting mutant gene DNA and pREP4 (Invitrogen, V004-50) encoding the lacI gene. The transformants were seeded onto agar containing ampicillin and kanamycin. The plasmids of interest were purified from the resulting clones. The mutations were confirmed to be introduced into the plasmids.

For plasmid construction requiring multistep mutation introduction, the procedure as described above was repeated to obtain plasmids into which mutations of interest were introduced. The combinations of the primers and template for such plasmid construction are listed in Table 4.

TABLE 3

| | position | position | position | | position | position | position |
|---|---|---|---|---|---|---|---|
| Name | 80 | 43 | 41 | Name | 43 | 45 | 85 |
| ValRS001(wt) | D | N | P | ValRS13-01 | G | T | Y |
| ValRS002 | | | A | ValRS13-02 | G | T | W |
| ValRS003 | | | G | ValRS13-03 | G | T | S |
| ValRS004 | | A | P | ValRS13-04 | G | T | M |
| ValRS005 | | | A | ValRS13-05 | G | T | K |
| ValRS006 | | | G | ValRS13-06 | G | T | N |
| ValRS007 | | S | P | ValRS13-07 | G | T | V |
| ValRS008 | | | A | ValRS13-08 | G | T | L |
| ValRS009 | | | G | ValRS13-09 | G | Y | A |
| ValRS010 | | V | P | ValRS13-10 | G | W | A |
| ValRS011 | | | A | ValRS13-11 | G | S | A |
| ValRS012 | | | G | ValRS13-12 | G | M | A |
| ValRS013 | | G | P | ValRS13-13 | G | K | A |
| ValRS014 | | | A | ValRS13-14 | G | N | A |
| ValRS015 | | | G | ValRS13-15 | G | V | A |
| ValRS016 | | D | P | ValRS13-16 | G | L | A |
| ValRS017 | | | A | | | | |
| ValRS018 | | | G | | | | |
| ValRS019 | | N | P | | | | |
| ValRS020 | | | A | | | | |
| ValRS021 | | | G | | | | |
| ValRS022 | | A | P | | | | |
| ValRS023 | | | A | | | | |
| ValRS024 | | | G | | | | |
| ValRS025 | | S | P | | | | |
| ValRS026 | | | A | | | | |
| ValRS027 | A | | G | | | | |
| ValRS028 | | V | P | | | | |
| ValRS029 | | | A | | | | |
| ValRS030 | | | G | | | | |
| ValRS031 | | G | P | | | | |
| ValRS032 | | | A | | | | |
| ValRS033 | | | G | | | | |
| ValRS034 | | D | P | | | | |
| ValRS035 | | | A | | | | |
| ValRS036 | | | G | | | | |
| ValRS037 | | D | P | | | | |
| ValRS038 | S | | A | | | | |
| ValRS039 | | | G | | | | |
| ValRS040 | | D | P | | | | |
| ValRS041 | V | | A | | | | |
| ValRS042 | | | G | | | | |
| ValRS043 | | D | P | | | | |
| ValRS044 | G | | A | | | | |
| ValRS045 | | | G | | | | |
| ValRS046 | S | N | P | | | | |
| ValRS047 | V | N | P | | | | |
| ValRS048 | G | N | P | | | | |

TABLE 4

| | forward | reverse | | | forward | reverse | |
|---|---|---|---|---|---|---|---|
| | primer | primer | Template | | primer | primer | Template |
| ValRS001 | | | | ValRS13-01 | F. V13-01 | R. V13-01 | ValRS13 |
| ValRS002 | F. V2 | R. V2 | wild type | ValRS13-02 | F. V13-02 | R. V13-02 | ValRS13 |
| ValRS003 | F. V3 | R. V3 | wild type | ValRS13-03 | F. V13-03 | R. V13-03 | ValRS13 |
| ValRS004 | F. V4 | R. V4 | wild type | ValRS13-04 | F. V13-04 | R. V13-04 | ValRS13 |
| ValRS005 | F. V5 | R. V5 | wild type | ValRS13-05 | F. V13-05 | R. V13-05 | ValRS13 |
| ValRS006 | F. V6 | R. V6 | wild type | ValRS13-06 | F. V13-06 | R. V13-06 | ValRS13 |
| ValRS007 | F. V7 | R. V7 | wild type | ValRS13-07 | F. V13-07 | R. V13-07 | ValRS13 |
| ValRS008 | F. VB | R. V8 | wild type | ValRS13-08 | F. V13-08 | R. V13-08 | ValRS13 |
| ValRS009 | F. V9 | R. V9 | wild type | ValRS13-09 | F. V13-09 | R. V13-09 | ValRS13 |
| ValRS010 | F. V10 | R. V10 | wild type | ValRS13-10 | F. V13-10 | R. V13-18 | ValRS13 |
| ValRS011 | F. V11 | R. V11 | wild type | ValRS13-11 | F. V13-11 | R. V13-11 | ValRS13 |
| ValRS012 | F. V12 | R. V12 | wild type | ValRS13-12 | F. V13-12 | R. V13-12 | ValRS13 |
| ValRS013 | F. V13 | R. V13 | wild type | ValRS13-13 | F. V13-13 | R. V13-13 | ValRS13 |
| ValRS014 | F. V14 | R. V14 | wild type | ValRS13-14 | F. V13-14 | R. V13-14 | ValRS13 |
| ValRS015 | F. V15 | R. V15 | wild type | ValRS13-15 | F. V13-15 | R. V13-15 | ValRS13 |
| ValRS016 | F. V16 | R. V16 | wild type | ValRS13-16 | F. V13-16 | R. V13-16 | ValRS13 |
| ValRS017 | F. V17 | R. V17 | wild type | | | | |
| ValRS018 | F. V18 | R. V18 | wild type | | | | |
| ValRS019 | F. V19 | R. V19 | wild type | | | | |
| ValRS020 | F. V2 | R. V2 | ValRS019 | | | | |
| ValRS021 | F. V3 | R. V3 | ValRS019 | | | | |
| ValRS022 | F. V4 | R. V4 | ValRS019 | | | | |
| ValRS023 | F. V5 | R. V5 | ValRS019 | | | | |
| ValRS024 | F. V6 | R. V6 | ValRS019 | | | | |
| ValRS025 | F. V7 | R. V7 | ValRS019 | | | | |
| ValRS026 | F. V8 | R. V8 | ValRS019 | | | | |
| ValRS027 | F. V9 | R. V9 | ValRS019 | | | | |
| ValRS028 | F. V10 | R. V10 | ValRS019 | | | | |
| ValRS029 | F. V11 | R. V11 | ValRS019 | | | | |
| ValRS030 | F. V12 | R. V12 | ValRS019 | | | | |
| ValRS031 | F. V13 | R. V13 | ValRS019 | | | | |
| ValRS032 | F. V14 | R. V14 | ValRS019 | | | | |
| ValRS033 | F. V15 | R. V15 | ValRS019 | | | | |
| ValRS034 | F. V16 | R. V16 | ValRS019 | | | | |
| ValRS035 | F. V17 | R. V17 | ValRS019 | | | | |
| ValRS036 | F. V18 | R V18 | ValRS019 | | | | |
| ValRS037 | F. V16 | R. V16 | ValRS046 | | | | |
| ValRS038 | F. V17 | R. V17 | ValRS046 | | | | |
| ValRS039 | F. V18 | R. V18 | ValRS046 | | | | |
| ValRS040 | F. V16 | R. V16 | ValRS047 | | | | |
| ValRS041 | F. V17 | R. V17 | ValRS047 | | | | |
| ValRS042 | F. V18 | R. V18 | ValRS047 | | | | |
| ValRS043 | F. V16 | R. V16 | ValRS048 | | | | |
| ValRS044 | F. V17 | R. V17 | ValRS048 | | | | |
| ValRS045 | F. V18 | R. V18 | ValRS048 | | | | |
| ValRS046 | F. V46 | R. V46 | wt | | | | |
| ValRS047 | F. V47 | R. V47 | wt | | | | |
| ValRS048 | F. V48 | R. V48 | wt | | | | |

### Small Scale Expression of Wild-Type and Mutant ValRSs

Next, a resulting mutant plasmid was introduced into E. coli, and the mutant protein was expressed. First, E. coli BL21 strain transformed with the mutant plasmid and pREP4 (Invitrogen, V004-50) was cultured at 37° C. in 4 mL of LB medium containing kanamycin and ampicillin. Then, when the OD value at 600 nm reached 0.4 to 0.8, IPTG was added to a final concentration of 0.5 mM. After further culturing at 37° C. for 4 hours, the bacterial pellets were collected using a centrifuge.

### Small Scale Purification of Wild-Type and Mutant ValRSs

Next, the resulting bacterial pellets were disrupted, and the mutant protein of interest was purified from the supernatant. Specifically, the bacterial pellets as described above were suspended in 600 μL of CHAPS solution (0.5% CHAPS (DOJINDO: 349-04722), 50% TBS (TaKaRa, T903)) and mixed with 6 μL of 30 U/μl rLysozyme (Novagen, 71110-3) followed by incubation at room temperature for 10 minutes. The reaction was further mixed with 2 μL of 2.5 U/μL benzonase nuclease (Novagen, 70746-3) followed by incubation at room temperature for 20 minutes, and an insoluble fraction was separated by centrifugation. The mutant protein was then purified from the resulting supernatant using QIAGEN Ni-NTA spin column kit (Qiagen, 31314) according to the product manual. Finally, excess imidazole was removed using a desalting column, PD miniTrap G-25 (GE Healthcare, 28-9180-07) according to the product manual.

Aminoacylation Reaction with N-methylvaline Using Wild-Type and Mutant ValRSs
Synthesis of E. coli tRNAVal by In Vitro Transcription Reaction

E. coli tRNA (R-tRNAVal2A (SEQ ID NO: 118)) was synthesized from a template DNA (D-tRNAVal2A (SEQ ID NO: 117)) by in vitro transcription reaction using RiboMAX Large Scale RNA production System T7 (Promega, P1300) in the presence of 7.5 mM GMP, and purified using RNeasy Mini kit (Qiagen).

D-tRNAVal2A
tRNAVal2A DNA sequence:
(SEQ ID NO: 117)
GGCGTAATACGACTCACTATAGCGTCCGTAGCTCAGTTGGTTAGAGCACC
ACCTTGACATGGTGGGGGTCGGTGGTTCGAGTCCACTCGGACGCACCA
R-tRNAVal2A
tRNAVal2A RNA sequence:
(SEQ ID NO: 118)
GCGUCCGUAGCUCAGUUGGUUAGAGCACCACCUUGACAUGGUGGGGGUCG
GUGGUUCGAGUCCACUCGGACGCACCA

### Aminoacylation Reaction

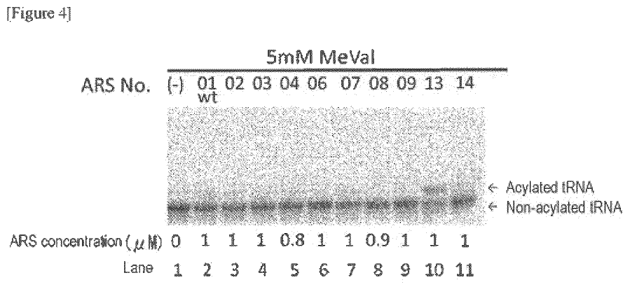

For the aminoacylation reaction, the solution containing 40 μM transcribed tRNA, 10 mM HEPES-K (pH 7.6), and 10 mM KCl solution was heated at 95° C. for 2 minutes and then left at room temperature for 5 minutes or more to refold the tRNA. This tRNA solution was added to a final concentration of 10 μM to an acylation buffer (in final concentrations of 50 mM HEPES-K [pH 7.6], 2 mM ATP, 100 mM potassium acetate, 10 mM magnesium acetate, 1 mM DTT, 2 mM spermidine, 0.1 mg/mL Bovine Serum Albumin), mixed with wild-type or mutant ValRS (final concentration 0.2 μM-1 μM) and N-methylvaline (final concentration 5 mM), and incubated at 37° C. for 10 minutes. Four volumes of a loading buffer (90 mM sodium acetate [pH 5.2], 10 mM EDTA, 95% (w/w) formamide, 0.001% (w/v) xylene cyanol) were added to the reaction solution and analyzed with acidic PAGE containing 6 M urea, and aminoacylation activity was detected by separating unreacted tRNA and aminoacyl-tRNA. RNA was stained with SYBR Gold (Life Technologies) and detected with LAS4000 (GE Healthcare) (FIG. 4).

As a result, tRNA acylated with N-methylvaline was observed when mutant 13 was used. It was demonstrated that mutant 13 had increased activity for aminoacylation with N-methylvaline compared to wild-type ValRS (FIG. 4, lane 2 vs 10).

### Translational Introduction of N-methylvaline Using Wild-Type and Mutant ValRSs

Template mRNAs for translation (R-V, R-V2, and R-V3 (SEQ ID NOs: 120, 122, and 124, respectively)) were synthesized from template DNAs (D-V, D-V2, and D-V3 (SEQ ID NOs: 119, 121, and 123, respectively)) by in vitro transcription reaction using RiboMAX Large Scale RNA production System T7 (Promega, P1300) and purified using RNeasy Mini kit (Qiagen).

D-V
DNA sequence:
(SEQ ID NO: 119)
GGCGTAATACGACTCACTATAGGGTTAACTTTAACAAGGAGAAAAACATG
CGTGTCCGTGACTACAAGGACGACGACGACAAGTAAGCTTCG
R-V
RNA sequence:
(SEQ ID NO: 120)
GGGUUAACUUUAACAAGGAGAAAAACAUGCGUGUCCGUGACUACAAGGAC
GACGACGACAAGUAAGCUUCG
D-V2
DNA sequence:
(SEQ ID NO: 121)
GGCGTAATACGACTCACTATAGGGTTAACTTTAACAAGGAGAAAAACATG
CGTGTCGTCCGTGACTACAAGGACGACGACGACAAGTAAGCTTCG
R-V2
RNA sequence:
(SEQ ID NO: 122)
GGGUUAACUUUAACAAGGAGAAAAACAUGCGUGUCGUCCGUGACUACAAG
GACGACGACGACAAGUAAGCUUCG
D-V3
DNA sequence:
(SEQ ID NO: 123)
GGCGTAATACGACTCACTATAGGGTTAACTTTAACAAGGAGAAAAACATG
CGTGTCGTCGTCCGTGACTACAAGGACGACGACGACAAGTAAGCTTCG
R-V3
RNA sequence:
(SEQ ID NO: 124)
GGGUUAACUUUAACAAGGAGAAAAACAUGCGCGUGUCGUCGUCUGACUAC
AAGGACGACGACGACAAGUAAGCUUCG

### Cell-Free Translation System

In order to confirm translational introduction of N-methylvaline, a desired polypeptide containing N-methylvaline was ribosomally synthesized by adding an N-methylvaline and a mutant ValRS to a cell-free translation system. The translation system used was PURE system, a reconstituted cell-free protein synthesis system from E. coli. Specifically, wild-type or mutant ValRS and N-methylvaline were added to a solution containing a basic cell-free translation solution (1 mM GTP, 1 mM ATP, 20 mM creatine phosphate, 50 mM HEPES-KOH pH 7.6, 100 mM potassium acetate, 9 mM magnesium acetate, 2 mM spermidine, 1 mM dithiothreitol, 1.5 mg/ml E. coli MRE600 (RNase negative)-derived tRNA (Roche), 0.1 mM 10-HCO—H4 folate, 4 μg/ml creatine kinase, 3 μg/ml myokinase, 2 unit/ml inorganic pyrophosphatase, 1.1 μg/ml nucleoside diphosphate kinase, 0.6 μM methionyl-tRNA transformylase, 0.26 μM EF-G, 0.24 μM RF2, 0.17 μM RF3, 0.5 μM RRF, 2.7 μM IF1, 0.4 μM IF2, 1.5 μM IF3, 40 μM EF-Tu, 84 μM EF-Ts, 1.2 μM ribosome, 0.03 μM ArgRS, 0.13 μM AspRS, 0.11 μM LysRS, 0.03 μM MetRS, 0.02 μM TyrRS (wherein the proteins prepared by the inventors were essentially prepared as His-tagged proteins)), and 1 μM template mRNA, each 250 μM arginine, aspartic acid, lysine, methionine, and tyrosine, and left at 37° C. for 1 hour to ribosomally synthesize the peptide.

### Detection by Mass Spectroscopy

Mass spectroscopy was performed using MALDI-TOF MS for detecting peptides into which N-methylvaline is ribosomally introduced. Specifically, a solution containing 1 μM template mRNA (R-V (SEQ ID NO: 120)) and arginine, lysine, methionine, tyrosine, and aspartic acid (each final concentration 250 μM) added to the cell-free translation system as described above was prepared. ValRS (final concentration 0.1 μM-1 μM) and N-methylvaline (final concentration 5 mM) were added to the solution and incubated at 37° C. for 60 minutes. The resulting translation reaction products were purified with SPE C-TIP (Nikkyo Technos Co., Ltd) and analyzed with MALDI-TOF MS. Translation products were identified with MALDI-TOF MS spectrometry using α-cyano-4-hydroxycinnamic acid as a matrix.

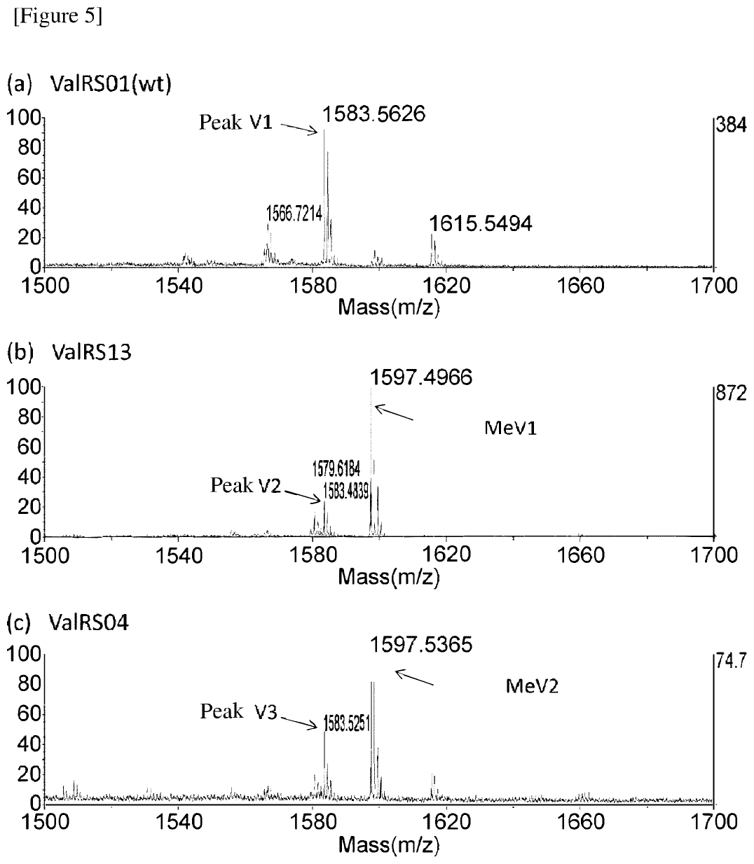

As a result of the translation using wild-type ValRS (SEQ ID NO: 24), a peak corresponding to the peptide sequence P-V1 introduced with valine contaminated in the cell-free translation system ((FIG. 5(a), Peak V1, m/z: [H+M]+=1583.6) was observed as main product. Similar experiments were performed using different mutant ValRSs. When ValRS04 (SEQ ID NO: 3) or ValRS13 (SEQ ID NO: 4) was used, a peak corresponding to the peptide sequence P-MeV1 with N-methylvaline introduced ((FIG. 5(b), Peak MeV1, m/z: [H+M]+=1597.5, FIG. 5(c), Peak MeV2, m/z: [H+M]+=1597.5) was observed as main product. This demonstrated, also from a view point of translation reaction that ValRS04 and ValRS13 have increased activity to N-methylvaline compared to wild-type ValRS. Although peaks corresponding to the peptide P-V1 introduced with valine contaminated in the translation system (FIG. 5(b) Peak V2, (c) Peak V3) were also observed at the same time, the intensity of the peak in using ValRS13 was weaker than that in using ValRS04. Therefore, it was suggested that ValRS13 has a higher activity to N-methylvaline (FIG. 5(b), (c)).

Peptide sequence P-V1
(SEQ ID NO: 125)
formylMetArgValArgAspTyrLysAspAspAspAspLys
Peptide sequence P-MeV1
(SEQ ID NO: 125)
formylMetArg[MeVal]ArgAspTyrLysAspAspAspAspLys

MALDI-TOF MS:

Calc. m/z: [H+M]+=1583.7 (the peptide corresponding to the sequence P-V1)

Calc. m/z: [H+M]+=1597.7 (the peptide corresponding to the sequence P-MeV1)

### Production of Mutant ValRS13-11 Having Further Increased Aminoacylation Activity to N-methylvaline

Improvement in activity to N-methylvaline was aimed by further introducing mutations into ValRS13 as described above. Plasmids into which the mutations of interest are introduced were prepared as mentioned above, expressed in E. coli, and purified to prepare mutant ValRSs (Table 3). As a result of screening by performing aminoacylation reaction and translational synthesis, ValRS13-11 (SEQ ID NO: 5) exhibited higher activity to N-methylvaline compared to ValRS13.

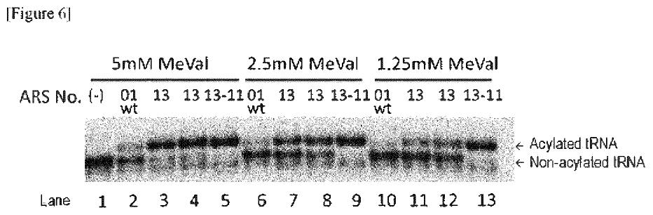

When aminoacylation reactions with N-methylvaline using wild-type ValRS, ValRS13, and ValRS13-11 were investigated, it was observed that the amount of aminoacyl-tRNA synthesized using ValRS13-11 was more than that synthesized using ValRS13 (FIG. 6, lane 8 vs lane 9 and lane 12 vs lane 13). Particularly, the amounts of aminoacyl-tRNA synthesized using ValRS13 and ValRS13-11 with N-methylvaline at the low concentration of 1.25 mM were greatly different, demonstrating that ValRS13-11 had a high activity.

Next, mass spectroscopy was performed using MALDI-TOF MS for confirming translational synthesis of peptides having N-methylvaline using ValRS13 and ValRS13-11. Specifically, a solution containing 1 μM template mRNA (R-V (SEQ ID NO: 120)) and arginine, lysine, methionine, tyrosine, and aspartic acid (each final concentration 250 μM) added to the cell-free translation system as described above was prepared. ValRS (final concentration 4 μM) and N-methylvaline (final concentration 5 mM) were added to the solution and incubated at 37° C. for 60 minutes. Template mRNAs encoding peptide sequences containing two consecutive or three consecutive N-methylvalines (R-V2 (SEQ ID NO: 122), R-V3 (SEQ ID NO: 124)) were used to perform similar experiments. The resulting translation reaction products were purified with SPE C-TIP (Nikkyo Technos Co., Ltd) and analyzed with MALDI-TOF MS.

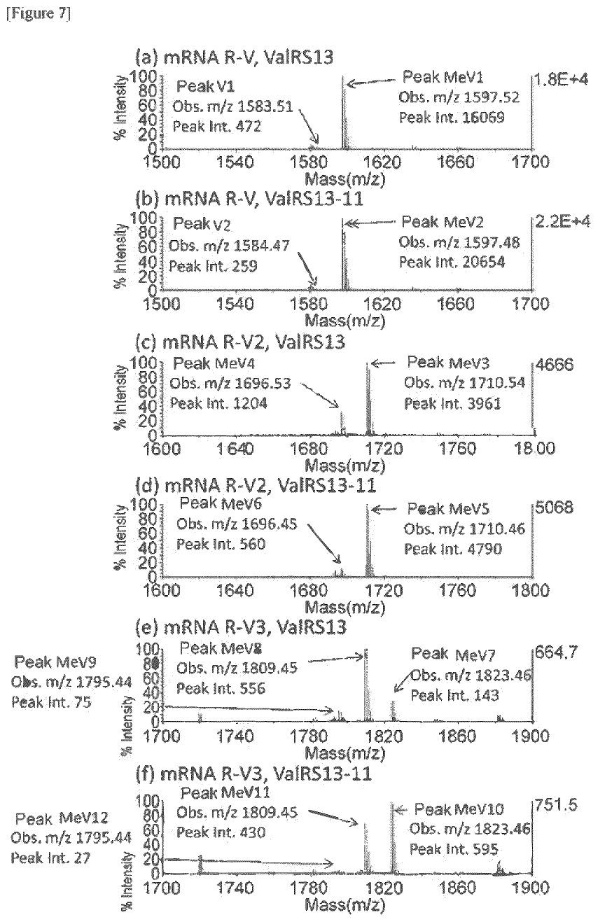

The intended synthesis of the peptide sequence P-MeV1 containing N-methylvaline using ValRS13 and ValRS13-11 was confirmed when the template mRNA containing one N-methylvaline (R-V (SEQ ID NO: 120)) was used (FIG. 7(a) Peak MeV1, (b) Peak MeV2). A peak corresponding to the peptide sequence P-V1 introduced with valine contaminated in the cell-free translation system was observed weakly (FIG. 7(a) Peak V1, (b) Peak V2).

Next, it was confirmed that the peptide sequence P-MeV2 containing two N-methylvaline residues was synthesized as main product by both the mutant ValRSs when a template mRNA containing two consecutive N-methylvalines (R-V2 (SEQ ID NO: 122)) was used (FIG. 7(c) Peak MeV3, (d) Peak MeV5). Meanwhile, the peptide sequence P-MeV4 containing one N-methylvaline residue and one valine residue (FIG. 7(c) Peak MeV4, (d) Peak MeV6) was observed, but the peak intensity was suppressed when using ValRS13-11 compared to ValRS13.

Finally, template mRNA containing three consecutive N-methylvalines (R-V3 (SEQ ID NO: 124)) was used to perform translation experiments. When ValRS13 was added, the synthesis of the target peptide sequence P-MeV3 containing three N-methylvaline residues was observed (FIG. 7(e), Peak MeV7), but the peptide sequence P-MeV5 comprising two N-methylvaline residues and one valine residue was produced as main product (FIG. 7(e), Peak MeV8). The peptide sequence P-MeV6 comprising one N-methylvaline residue and two valine residues was also observed (FIG. 7(e), Peak MeV9). Meanwhile, when ValRS13-11 was added, it was observed that the target peptide sequence P-MeV3 containing three N-methylvaline residues was ribosomally synthesized as main product (FIG. 7(f), Peak MeV10). The peptide sequences P-MeV5 and P-MeV6 containing valine were also observed, but the peak intensity of these peptide sequences was suppressed compared to that obtained when using ValRS13 (FIG. 7(f), Peak MeV11 and MeV12). These results indicate that ValRS13-11 has increased aminoacylation activity to N-methylvaline compared to ValRS13, leading to the increase of the ribosomally synthesized amount of the target peptide containing N-methylvaline.

Peptide sequence P-MeV2
(SEQ ID NO: 126)
formylMetArg[MeVal][MeVal]ArgAspTyrLysAspAspAspAsp
Lys
Peptide sequence P-MeV4
(SEQ ID NO: 126)
formylMetArg[MeVal]ValArgAspTyrLysAspAspAspAspLys
or
formylMetArgVal[MeVal]ArgAspTyrLysAspAspAspAspLys
Peptide sequence P-MeV3
(SEQ ID NO: 127)
formylMetArg[MeVal][MeVal][MeVal]ArgAspTyrLysAsp
AspAspAspLys
Peptide sequence P-MeV5
(SEQ ID NO: 127)
formylMetArg[MeVal][MeVal]ValArgAspTyrLysAspAsp
AspAspLys
or
formylMetArg[MeVal]Val[MeVal]ArgAspTyrLysAspAsp
AspAspLys
or
formylMetArgVal[MeVal][MeVal]ArgAspTyrLysAspAsp
AspAspLys
Peptide sequence P-MeV6
(SEQ ID NO: 127)
formylMetArg[MeVal]ValValArgAspTyrLysAspAspAspAsp
Lys
or
formylMetArgVal[MeVal]ValArgAspTyrLysAspAspAspAsp
Lys
or
formylMetArgValVal[MeVal]ArgAspTyrLysAspAspAspAsp
Lys

MALDI-TOF MS:

Calc. m/z: [H+M]+=1710.8 (the peptide corresponding to the sequence P-MeV2)

Calc. m/z: [H+M]+=1696.8 (the peptide corresponding to the sequence P-MeV4)

Calc. m/z: [H+M]+=1823.9 (the peptide corresponding to the sequence P-MeV3)

Calc. m/z: [H+M]+=1809.9 (the peptide corresponding to the sequence P-MeV5)

Calc. m/z: [H+M]+=1795.9 (the peptide corresponding to the sequence P-MeV6)

### Example 3: Development of N-methylserine-accepting ARS

### Preparation of Plasmids for Wild-Type and Mutant SerRSs

The mutant SerRS plasmids (having His-tag (6×His) at the N-terminus) listed in Table 5 were constructed by introducing site-directed mutations using PCR into a plasmid comprising the ORF sequence (SEQ ID NOs: 25, 26) of wild-type SerRS gene of E. coli (PQE-32(2) 2_wtSERRS), which was used as starting material. Specifically, 2.5 μL of 10 ng/μL template, 12.5 μL of 2×KOD Fx buffer (TOYOBO, KFX-101), 0.75 μL of 10 μM forward primer, 0.75 μL of 10 μM reverse primer, 5 μL of 2 mM dNTP, 0.5 μL of KOD FX (TOYOBO, KFX-101), and 3 μL of H₂O were mixed together. Next, the resulting reaction solution was heated at 94° C. for 2 minutes and then subjected to 10 cycles, each consisting of heating at 98° C. for 10 seconds and heating at 68° C. for 7 minutes, to amplify the mutant gene. The combinations of the template plasmid, forward primer, and reverse primer used are listed in Table 6. Each sequence of primers “F.S2” through “F.S8”, “F.S15” through “F.S23”, and “F.S33” through “F.S38” corresponds to SEQ ID NOs: 128-149 in ascending order. Each sequence of primers “R.S2” through “R.S8”, “R.S15” through “R.S23”, and “R.S33” through “R.S38” corresponds to SEQ ID NOs: 150-171 in ascending order. 0.5 μL of 10 U/μL DpnI was then added to the PCR reaction solution and further incubated at 37° C. for 1.5 hours to digest the template DNA, and the resulting mutant DNA was purified. E. coli XL-1 Blue strain (STRATAGENE, 200236) was then co-transformed with the resulting mutant gene DNA and pREP4 (Invitrogen, V004-50) encoding the lacI gene. The transformants were seeded onto agar containing ampicillin and kanamycin. The plasmids of interest were purified from the resulting clones. The mutations were confirmed to be introduced into the plasmids.

For plasmid construction requiring multistep mutation introduction, the procedure as described above was repeated to obtain plasmids into which mutations of interest were introduced. The combinations of the primers and template for such plasmid construction are listed in Table 6.

TABLE 5

| Name | position 237 | position 239 | position 389 |
|---|---|---|---|
| SerRS01(WT) | T | E | N |
| SerRS02 | A | E | N |
| SerRS03 | S | | |
| SerRS04 | G | | |
| SerRS05 | T | A | N |
| SerRS06 | | V | |
| SerRS07 | | S | |
| SerRS08 | T | E | A |
| SerRS09 | A | E | A |
| SerRS10 | S | | |
| SerRS11 | G | | |
| SerRS12 | T | A | |
| SerRS13 | | V | |
| SerRS14 | | S | |
| SerRS15 | A | A | N |
| SerRS16 | | V | |
| SerRS17 | | S | |
| SerRS18 | S | A | |
| SerRS19 | | V | |
| SerRS20 | | S | |
| SerRS21 | G | A | |
| SerRS22 | | V | |
| SerRS23 | | S | |
| SerRS24 | A | A | A |
| SerRS25 | | V | |
| SerRS26 | | S | |
| SerRS27 | S | A | |
| SerRS28 | | V | |
| SerRS29 | | S | |
| SerRS30 | G | A | |
| SerRS31 | | V | |
| SerRS32 | | S | |
| SerRS33 | E291A | | |
| SerRS34 | K289A | | |
| SerRS35 | T | G | N |
| SerRS36 | T | D | N |
| SerRS37 | S | G | N |
| SerRS38 | S | D | N |

TABLE 6

| | forward primer | revers primer | Template |
|---|---|---|---|
| SerRS001 | | | |
| SerRS002 | F. S2 | R. S2 | wt |
| SerRS003 | F. S3 | R. S3 | wt |
| SerRS004 | F. S4 | R. S4 | wt |
| SerRS005 | F. S5 | R. S5 | wt |
| SerRS006 | F. S6 | R. S6 | wt |
| SerRS007 | F. S7 | R. S7 | wt |
| SerRS008 | F. S8 | R. S8 | wt |
| SerRS009 | F. S2 | R. S2 | SerRS008 |
| SerRS010 | F. S3 | R. S3 | SerRS008 |
| SerRS011 | F. S4 | R. S4 | SerRS008 |
| SerRS012 | F. S5 | R. S5 | SerRS008 |
| SerRS013 | F. S6 | R. S6 | SerRS008 |
| SerRS014 | F. S7 | R. S7 | SerRS008 |
| SerRS015 | F. S15 | R. S15 | wt |
| SerRS016 | F. S16 | R. S16 | wt |
| SerRS017 | F. S17 | R. S17 | wt |
| SerRS018 | F. S18 | R. S18 | wt |
| SerRS019 | F. S18 | R. S19 | wt |
| SerRS020 | F. S20 | R. S20 | wt |
| SerRS021 | F. S21 | R. S21 | wt |
| SerRS022 | F. S22 | R. S22 | wt |
| SerRS023 | F. S23 | R. S23 | wt |
| SerRS024 | F. S15 | R. S15 | SerRS008 |
| SerRS025 | F. S16 | R. S16 | SerRS008 |
| SerRS026 | F. S17 | R. S17 | SerRS008 |
| SerRS027 | F. S18 | R. S18 | SerRS008 |
| SerRS028 | F. S19 | R. S19 | SerRS008 |
| SerRS029 | F. S20 | R. S20 | SerRS008 |
| SerRS030 | F. S21 | R. S21 | SerRS008 |
| SerRS031 | F. S22 | R. S22 | SerRS008 |
| SerRS032 | F. S23 | R. S23 | SerRS008 |
| SerRS033 | F. S33 | R. S33 | wt |
| SerRS034 | F. S34 | R. S34 | wt |
| SerRS035 | F. S35 | R. S35 | wt |
| SerRS036 | F. S36 | R. S36 | wt |
| SerRS037 | F. S37 | R. S37 | wt |
| SerRS038 | F. S38 | R. S38 | wt |

### Small Scale Expression of Wild-Type and Mutant SerRSs

Next, the resulting mutant plasmid was introduced into E. coli to express the mutant protein. First, E. coli BL21 strain transformed with the mutant plasmid and pREP4 (Invitrogen, V004-50) was cultured at 37° C. in 4 mL of LB medium containing kanamycin and ampicillin. Then, when the OD value at 600 nm reached 0.4 to 0.8, IPTG was added to a final concentration of 0.5 mM. After further culturing at 37° C. for 4 hours, the bacterial pellets were collected with a centrifuge.

### Small Scale Purification of Wild-Type and Mutant SerRSs

Next, the resulting bacterial pellets were disrupted, and the mutant protein of interest was purified from the supernatant. Specifically, the bacterial pellets as described above were suspended in 600 μL of CHAPS solution (0.5% CHAPS (DOJINDO: 349-04722), 50% TBS (TaKaRa, T903)) and mixed with 6 μL of 30 U/μl rLysozyme (Novagen, 71110-3) followed by incubation at room temperature for 10 minutes. The reaction was further mixed with 2 μL of 2.5 U/μL benzonase nuclease (Novagen, 70746-3) followed by incubation at room temperature for 20 minutes, and an insoluble fraction was separated by centrifugation. Then, the mutant protein was purified from the resulting supernatant using QIAGEN Ni-NTA spin column kit (Qiagen, 31314) according to the product manual. Finally, excess imidazole was removed using a desalting column, PD miniTrap G-25 (GE Healthcare, 28-9180-07) according to the product manual.

Aminoacylation Reaction with N-methylserine Using Wild-Type and Mutant SerRSs
Synthesis of E. coli tRNASer by In Vitro Transcription Reaction

E. coli tRNA (R-tRNASer3 (SEQ ID NO: 173)) was synthesized from a template DNA (D-tRNASer3 (SEQ ID NO: 172)) by in vitro transcription reaction using RiboMAX Large Scale RNA production System T7 (Promega, P1300) in the presence of 7.5 mM GMP, and purified using RNeasy Mini kit (Qiagen).

D-tRNASer3
tRNASer3 DNA sequence:
(SEQ ID NO: 172)
GGCGTAATACGACTCACTATAGGTGAGGTGGCCGAGAGGCTGAAGGCGCT
CCCCTGCTAAGGGAGTATGCGGTCAAAAGCTGCATCCGGGGTTCGAATCC
CCGCCTCACCGCCA
R-tRNASer3
tRNASer3 RNA sequence:
(SEQ ID NO: 173)
GGUGAGGUGGCCGAGAGGCUGAAGGCGCUCCCCUGCUAAGGGAGUAUGCG
GUCAAAAGCUGCAUCCGGGGUUCGAAUCCCCGCCUCACCGCCA

### Aminoacylation Reaction

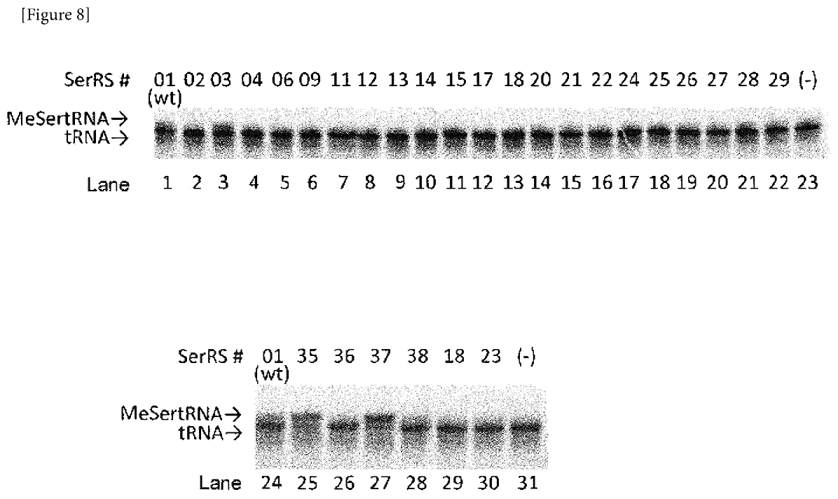

For the aminoacylation reaction, the solution containing 40 μM transcribed tRNA, 10 mM HEPES-K (pH 7.6), and 10 mM KCl solution was heated at 95° C. for 2 minutes and then left at room temperature for 5 minutes or more to refold the tRNA. This tRNA solution was added to a final concentration of 10 μM to an acylation buffer (final concentration 50 mM HEPES-K [pH 7.6], 2 mM ATP, 100 mM potassium acetate, 10 mM magnesium acetate, 1 mM DTT, 2 mM spermidine, 0.1 mg/mL Bovine Serum Albumin), mixed with wild-type or mutant SerRS (final concentration 0.1 μM-2 μM) and N-methylserine (final concentration 1 mM), and incubated at 37° C. for 10 minutes. Four volumes of a loading buffer (90 mM sodium acetate [pH 5.2], 10 mM EDTA, 95% (w/w) formamide, 0.001% (w/v) xylene cyanol) were added to the reaction solution and analyzed with acidic PAGE containing 6 M urea, and aminoacylation activity was detected by separating unreacted tRNA and aminoacyl-tRNA. RNA was stained with SYBR Gold (Life Technologies) and detected with LAS4000 (GE Healthcare) (FIG. 8).

As a result, tRNA acylated with N-methylserine was observed when the mutants 03 (SEQ ID NO: 6), 35 (SEQ ID NO: 8), and 37 (SEQ ID NO: 9) were used. It is suggested that these mutants had increased activity for aminoacylation with N-methylserine compared to wild-type SerRS (FIG. 8, lanes 3, 25, 27).

### Translational Introduction of N-methylserine Using Wild-Type and Mutant SerRSs

Template mRNA (R-S(SEQ ID NO: 175)) was synthesized from a template DNA (D-S (SEQ ID NO: 174)) by in vitro transcription reaction using RiboMAX Large Scale RNA production System T7 (Promega, P1300) and purified using RNeasy Mini kit (Qiagen).

D-S (CT21) (SEQ ID NO: 174) DNA sequence:
GGCGTAATACGACTCACTATAGGGTTAACTTTAACAAGGAGA
AAAACATGCGTTCCCGTGACTACAAGGACGACGACGACAAGTAAGCTTCG
R-S (SEQ ID NO: 175) RNA sequence:
GGGUUAACUUUAACAAGGAGAAAAACAUGCGUUCCCGUG
ACUACAAGGACGACGACGACAAGUAAGCUUCG

### Cell-Free Translation System

In order to confirm translational introduction of N-methylserine, a desired polypeptide containing N-methylserine was ribosomally synthesized by adding N-methylserine and SerRS to a cell-free translation system. The translation system used was PURE system, a reconstituted cell-free protein synthesis system from E. coli. Specifically, wild-type or mutant SerRS and N-methylserine were added to a solution containing a basic cell-free translation solution (1 mM GTP, 1 mM ATP, 20 mM creatine phosphate, 50 mM HEPES-KOH pH 7.6, 100 mM potassium acetate, 9 mM magnesium acetate, 2 mM spermidine, 1 mM dithiothreitol, 1.5 mg/ml E. coli MRE600 (RNase negative)-derived tRNA (Roche), 0.1 mM 10-HCO—H4 folate, 4 μg/ml creatine kinase, 3 μg/ml myokinase, 2 unit/ml inorganic pyrophosphatase, 1.1 μg/ml nucleoside diphosphate kinase, 0.6 μM methionyl-tRNA transformylase, 0.26 μM EF-G, 0.24 μM RF2, 0.17 μM RF3, 0.5 μM RRF, 2.7 μM IF1, 0.4 μM IF2, 1.5 μM IF3, 40 μM EF-Tu, 84 μM EF-Ts, 1.2 μM ribosome, 0.03 μM ArgRS, 0.13 μM AspRS, 0.11 μM LysRS, 0.03 μM MetRS, 0.02 μM TyrRS (wherein the proteins prepared by the inventors were essentially prepared as His-tagged proteins)), and 1 μM template mRNA, each 250 μM arginine, aspartic acid, lysine, methionine, and tyrosine, and left at 37° C. for 1 hour to ribosomally synthesize the peptide.

### Detection by Mass Spectroscopy

Mass spectroscopy was performed using MALDI-TOF MS for detecting peptides into which N-methylserine is ribosomally introduced. Specifically, a solution containing 1 μM template mRNA (R-S(SEQ ID NO: 175)) and arginine, lysine, methionine, tyrosine, and aspartic acid (each final concentration 250 μM) added to the cell-free translation system as described above was prepared. SerRS (final concentration 0.1-2 μM) and N-methylserine (final concentration 5 mM) were added to the solution and incubated at 37° C. for 60 minutes. The resulting translation reaction products were purified with SPE C-TIP (Nikkyo Technos Co., Ltd) and analyzed with MALDI-TOF MS. Translation products were identified with MALDI-TOF MS spectrometry using a-cyano-4-hydroxycinnamic acid as a matrix.

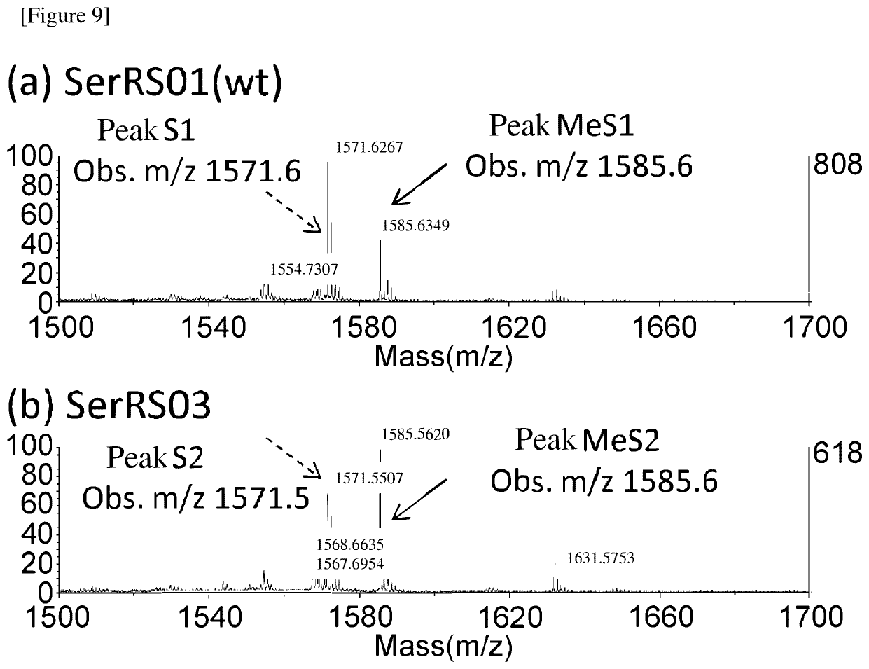

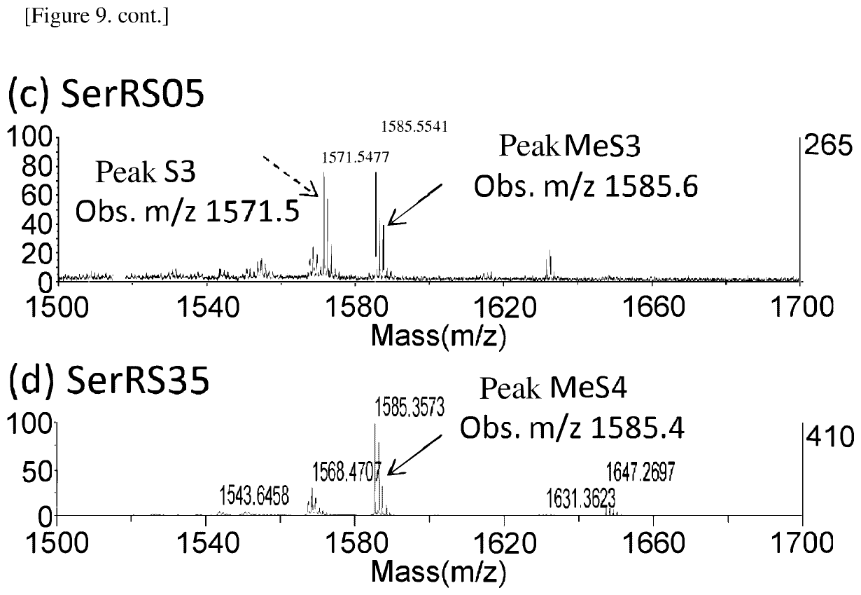

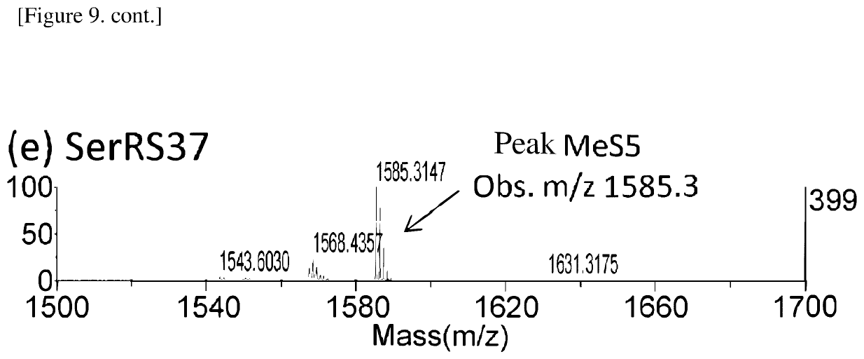

As a result of the translation using wild-type SerRS (SEQ ID NO: 25)), a peak corresponding to the target peptide peak P-CT21MeSer containing N-methylserine (FIG. 9(a), Peak MeS1 m/z: [H+M]+=1585.6) was observed, but the peak corresponding to Ser-containing peptide P-CT21Ser probably derived from Ser contaminated in the translation system in trace amounts (FIG. 9(a), Peak S1 m/z: [H+M]+=1571.6) was observed as main product (FIG. 9(a)). On the other hand, when similar experiments were performed using the modified SerRS with mutations introduced (Ser03 (SEQ ID NO: 6), 05 (SEQ ID NO: 7)), the peak corresponding to P-CT21Ser (FIG. 9(b) Peak S2, (c) Peak S3) was observed similarly but as a side product, and it was revealed that the main product was P-CT21MeSer (FIG. 9(b) Peak MeS2, (c) Peak MeS3). Particularly, when SerRS35 and 37 were used, it was demonstrated that the peak corresponding to P-CT21Ser was not observed and CT21MeSer was synthesized with high purity (FIG. 9(d) Peak MeS4, (e) Peak MeS5). Consequently, these modified SerRSs were demonstrated to have increased activity to MeSer compared to wild-type SerRS.

Peptide sequence P-CT21Ser
(SEQ ID NO: 176)
formylMetArgSerArgAspTyrLysAspAspAspAspLys
Peptide sequence P-CT21MeSer
(SEQ ID NO: 176)
formylMetArg[MeSer]ArgAspTyrLysAspAspAspAspLys

MALDI-TOF MS:

Calc. m/z: [H+M]+=1571.7 (the peptide corresponding to the sequence P-CT21Ser)

Calc. m/z: [H+M]+=1585.7 (the peptide corresponding to the sequence P-CT21MeSer)

### Example 4: Development of N-methylthreonine-accepting ARS

### Preparation of Wild-Type and Mutant ThrRS Proteins

Expression vectors having a polyhistidine sequence at the N-terminus and comprising mutations listed in Table 7 were constructed. Subsequently, an expression strain was transformed with the vectors, and the mutant proteins of interest were purified with a nickel column from supernatants obtained by disrupting cells.

TABLE 7

| | Position | Position | Position | Position | Position | Position |
|---|---|---|---|---|---|---|
| Name | 332 | 334 | 385 | 462 | 484 | 511 |
| ThrRS01(wt) | M | C | H | Y | Q | H |
| ThrRS02 | A | C | H | Y | Q | H |
| ThrRS03 | G | C | H | Y | Q | H |
| ThrRS04 | M | A | H | Y | Q | H |
| ThrRS05 | M | G | H | Y | Q | H |
| ThrRS06 | M | C | A | Y | Q | H |
| ThrRS07 | M | C | H | A | Q | H |
| ThrRS08 | M | C | H | L | Q | H |
| ThrRS09 | M | C | H | V | Q | H |
| ThrRS10 | M | C | H | F | Q | H |
| ThrRS11 | M | C | H | Y | A | H |
| ThrRS12 | M | C | H | Y | N | H |
| ThrRS13 | M | C | H | Y | Q | A |
| ThrRS14 | M | C | H | Y | Q | G |

### Translational Introduction of N-methylthreonine Using Wild-Type and Mutant ThrRSs

A template mRNA (R-T (SEQ ID NO: 178)) was synthesized from a template DNA (D-T (SEQ ID NO: 177)) by in vitro transcription reaction using RiboMAX Large Scale RNA production System T7 (Promega, P1300) and purified using RNeasy Mini kit (Qiagen).

D-T (3lib15#09)
DNA sequence:
(SEQ ID NO: 177)
GGCGTAATACGACTCACTATAGGGTTAACTTTAAGAAGGAGATATACATA
TGAAGGCTGGTCCGGGTTTTATGACTAAGAGTGGTAGTGGTAGTTAAGCT
TCG
R-T
RNA sequence:
(SEQ ID NO: 178)
GGGUUAACUUUAAGAAGGAGAUAUACAUAUGAAGGCUGGUCCGGGUUUUA
UGACUAAGAGUGGUAGUGGUAGUUAAGCUUCG

### Cell-Free Translation System

In order to confirm translational introduction of N-methylthreonine, a desired polypeptide containing N-methylthreonine was ribosomally synthesized by adding N-methylthreonine and a mutant ThrRS to a cell-free translation system. The translation system used was PURE system, a reconstituted cell-free protein synthesis system from E. coli. Specifically, wild-type or mutant ThrRS and N-methylthreonine were added to a solution containing a basic cell-free translation solution (1 mM GTP, 1 mM ATP, 20 mM creatine phosphate, 50 mM HEPES-KOH pH 7.6, 100 mM potassium acetate, 9 mM magnesium acetate, 2 mM spermidine, 1 mM dithiothreitol, 1.5 mg/ml E. coli MRE600 (RNase negative)-derived tRNA (Roche), 0.1 mM 10-HCO—H4 folate, 4 μg/ml creatine kinase, 3 μg/ml myokinase, 2 unit/ml inorganic pyrophosphatase, 1.1 μg/ml nucleoside diphosphate kinase, 0.6 μM methionyl-tRNA transformylase, 0.26 μM EF-G, 0.24 μM RF2, 0.17 μM RF3, 0.5 μM RRF, 2.7 μM IF1, 0.4 μM IF2, 1.5 μM IF3, 40 μM EF-Tu, 93 μM EF-Ts, 1.2 μM ribosome, 2.73 μM AlaRS, 0.13 μM AspRS, 0.09 μM GlyRS, 0.11 μM LysRS, 0.03 μM MetRS, 0.68 μM PheRS, 0.16 μM ProRS, 0.25 μM SerRS (wherein the proteins prepared by the inventors were essentially prepared as His-tagged proteins)), and 1 μM template mRNA, each 250 μM glycine, proline, alanine, phenylalanine, lysine, methionine, and serine, and left at 37° C. for 1 hour to ribosomally synthesize the peptide.

### Detection by Mass Spectroscopy

Mass spectroscopy was performed using MALDI-TOF MS for detecting peptides into which N-methylthreonine is ribosomally introduced. Specifically, a solution containing 1 μM template mRNA (R-T (SEQ ID NO: 178)) and glycine, proline, alanine, phenylalanine, lysine, methionine, and serine (each final concentration 250 μM) added to the cell-free translation system as described above was prepared. ThrRS (final concentration 2 μM) and N-methylthreonine (final concentration 5 mM) were added to the solution and incubated at 37° C. for 60 minutes. The resulting translation reaction products were purified with SPE C-TIP (Nikkyo Technos Co., Ltd) and analyzed with MALDI-TOF MS. Translation products were identified with MALDI-TOF MS spectrometry using a-cyano-4-hydroxycinnamic acid as a matrix.

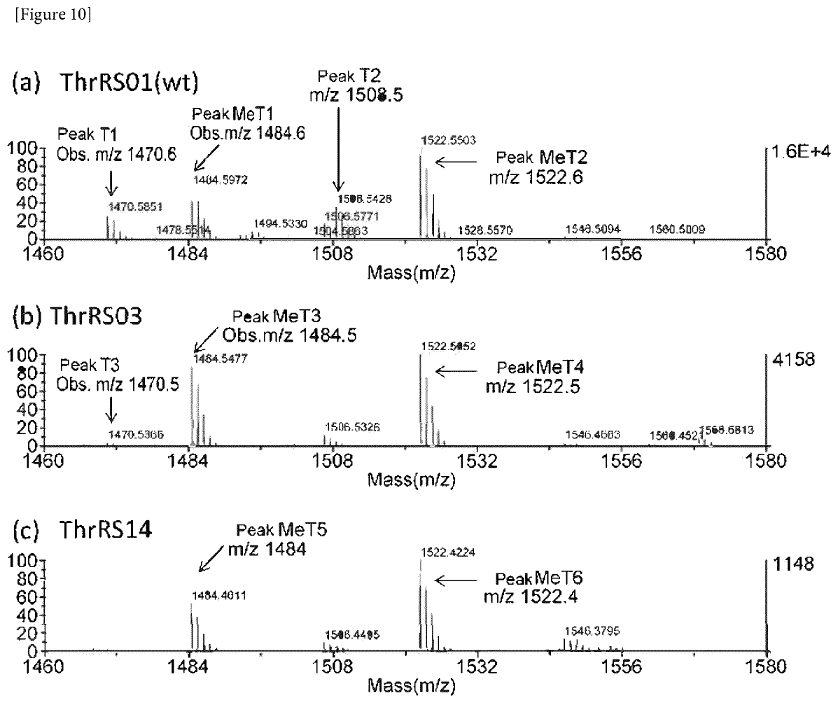

As a result of the translation using wild-type ThrRS (SEQ ID NO: 29), a peak corresponding to the peptide peak P-31ib15MeThr of interest containing N-methylthreonine ((FIG. 10(a), Peak MeT1) and a peak corresponding to a potassium salt of the peptide (FIG. 10(a), Peak MeT2) were observed as main products, but at the same time, a peak corresponding to the peptide P-31ib15Thr derived from Thr contaminated in the translation system in trace amounts (FIG. 10(a), PeakT1) and a peak corresponding to a potassium salt of the peptide (FIG. 10(a), PeakT2) were also observed, revealing that the purity of the translation products was adversely affected (FIG. 10(a)). On the other hand, when experiments were performed using the modified ThrRS 03 (SEQ ID NO: 10) and the modified ThrRS 14 (SEQ ID NO: 11) with mutations introduced, the peaks derived from P-CT21MeThr (Peak MeT3-6, FIG. 10) were similarly observed, and the peak corresponding to the peptide sequence P-31ib15Thr was only slightly observed (FIG. 10(b), (c)). In other words, it was demonstrated that the peptide with MeThr introduced was synthesized with higher purity using the modified ThrRSs compared to using wild-type ThrRS, and it was suggested that the modified ThrRS 03 and 14 are ARSs that can introduce MeThr into peptides more efficiently compared to wild-type ThrRS.

Peptide sequence P-3lib15Thr
(SEQ ID NO: 179)
formylMetLysAlaGlyProGlyPheMetThrLysSerGlySerGly
Ser
Peptide sequence P-3lib15MeThr
(SEQ ID NO: 179)
formylMetLysAlaGlyProGlyPheMet[MeThr]LysSerGlySer
GlySer

MALDI-TOF MS:

Calc. m/z: [H+M]+=1470.7, [K+M]+=1508.8 (the peptide corresponding to the sequence P-31ib15Thr)

Calc. m/z: [H+M]+=1484.7, [K+M]+=1522.8 (the peptide corresponding to the sequence P-31ib15MeThr)

### Example 5: Development of N-methyltryptophan-accepting ARS

### Preparation of Wild-Type and Mutant TrpRS Proteins

Expression vectors having a polyhistidine sequence at the N-terminus and containing mutations listed in Table 8 were constructed. Then, an expression strain was transformed with the vectors, and the mutant proteins of interest were purified with a nickel column from supernatants obtained by disrupting cells.

TABLE 8

| Name | Position 128 | Position 132 | Position 150 | Position 153 |
|---|---|---|---|---|
| ThrRS01(wt) | Y | M | Q | H |
| ThrRS02 | Y | M | Q | A |
| ThrRS03 | Y | M | A | H |
| ThrRS04 | Y | M | A | A |
| ThrRS05 | Y | A | Q | H |
| ThrRS06 | Y | A | Q | A |
| ThrRS07 | Y | A | A | H |
| ThrRS08 | Y | A | A | A |
| ThrRS09 | A | M | Q | H |
| ThrRS10 | Y | M | S | H |
| ThrRS11 | Y | M | G | H |
| ThrRS12 | Y | M | Q | G |
| ThrRS13 | Y | M | G | A |
| ThrRS14 | Y | M | A | G |
| ThrRS15 | Y | M | G | G |
| ThrRS16 | Y | G | A | A |
| ThrRS17 | Y | I | A | A |
| ThrRS18 | Y | V | A | A |
| ThrRS19 | Y | Q | A | A |
| ThrRS20 | Y | L | A | A |

### Translational Introduction of N-methyltryptophan Using Wild-Type and Mutant TrpRSs

A template mRNA(R-W (SEQ ID NO: 197)) was synthesized from a template DNA (D-W (SEQ ID NO: 196)) by in vitro transcription reaction using RiboMAX Large Scale RNA production System T7 (Promega, P1300) and purified using RNeasy Mini kit (Qiagen).

D-W (CT29)
DNA sequence:
(SEQ ID NO: 196)
GGCGTAATACGACTCACTATAGGGTTAACTTTAACAAGGAGAAAAACATG
CGTTGGCGTGACTACAAGGACGACGACGACAAGTAAGCTTCG
R-W
RNA sequence:
(SEQ ID NO: 197)
GGGUUAACUUUAACAAGGAGAAAAACAUGCGUUGGCGUGACUACAAGGAC
GACGACGACAAGUAAGCUUCG

### Cell-Free Translation System

In order to confirm translational introduction of N-methyltryptophan, a desired polypeptide containing N-methyltryptophan was ribosomally synthesized by adding N-methyltryptophan and a mutant TrpRS to a cell-free translation system. The translation system used was PURE system, a reconstituted cell-free protein synthesis system from E. coli. Specifically, wild-type or mutant TrpRS and N-methyltryptophan were added to a solution containing a basic cell-free translation solution (1 mM GTP, 1 mM ATP, 20 mM creatine phosphate, 50 mM HEPES-KOH pH 7.6, 100 mM potassium acetate, 9 mM magnesium acetate, 2 mM spermidine, 1 mM dithiothreitol, 1.5 mg/ml E. coli MRE600 (RNase negative)-derived tRNA (Roche), 0.1 mM 10-HCO—H4 folate, 4 μg/ml creatine kinase, 3 μg/ml myokinase, 2 unit/ml inorganic pyrophosphatase, 1.1 μg/ml nucleoside diphosphate kinase, 0.6 μM methionyl-tRNA transformylase, 0.26 μM EF-G, 0.24 μM RF2, 0.17 μM RF3, 0.5 μM RRF, 2.7 μM IF1, 0.4 μM IF2, 1.5 μM IF3, 40 μM EF-Tu, 84 μM EF-Ts, 1.2 μM ribosome, 0.03 μM ArgRS, 0.13 μM AspRS, 0.11 μM LysRS, 0.03 μM MetRS, 0.02 μM TyrRS (wherein the proteins prepared by the inventors were essentially prepared as His-tagged proteins)), and 1 μM template mRNA, each 250 μM arginine, aspartic acid, lysine, methionine, and tyrosine, and left at 37° C. for 1 hour to ribosomally synthesize the peptide.

### Detection by Mass Spectroscopy

Mass spectroscopy was performed using MALDI-TOF MS for detecting peptides into which N-methyltryptophan is ribosomally introduced. Specifically, a solution containing 1 μM template mRNA (R-W (SEQ ID NO: 197)) and arginine, lysine, methionine, tyrosine, and aspartic acid (each final concentration 250 μM) added to the cell-free translation system as described above was prepared. TrpRS (final concentration 5 μM) and N-methyltryptophan (final concentration 5 mM) were added to the solution and incubated at 37° C. for 60 minutes. The resulting translation reaction products were purified with SPE C-TIP (Nikkyo Technos Co., Ltd) and analyzed with MALDI-TOF MS. Translation products were identified with MALDI-TOF MS spectrometry using a-cyano-4-hydroxycinnamic acid as a matrix.

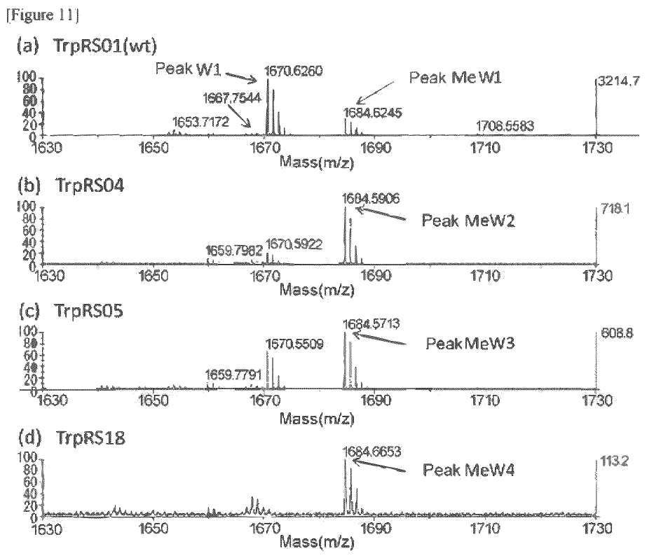

As a result of the translation using wild-type TrpRS (SEQ ID NO: 188), a peak corresponding to the target peptide peak P-CT29MeTrp containing N-methyltryptophan (FIG. 11(a), Peak MeW1) was observed, but a peak corresponding to peptide P-CT29Trp derived from Trp contaminated in the translation system in trace amounts was observed as main product (FIG. 11(a), PeakW1). On the other hand, when experiments were performed using the modified TrpRS 04 (SEQ ID NO: 184), the modified TrpRS 05 (SEQ ID NO: 185), and the modified TrpRS 18 (SEQ ID NO: 186) with mutations introduced, peaks derived from P-CT29MeTrp (Peak MeW2-4, FIG. 11(b)-(d)) were observed as main product. In other words, it is demonstrated that the peptide introduced with MeTrp was synthesized at a higher purity using modified TrpRSs compared to using wild-type TrpRS, and it was suggested that these modified TrpRSs are ARSs that can introduce MeTrp into peptides more efficiently compared to wild-type TrpRS.

Peptide sequence P-CT29Trp
(SEQ ID NO: 198)
formylMetArgTrpArgAspTyrLysAspAspAspAspLys
Peptide sequence P-CT29MeTrp
(SEQ ID NO: 199)
formylMetArg[MeTrp]ArgAspTyrLysAspAspAspAspLys

MALDI-TOF MS:

Calc. m/z: [H+M]+=1670.7 (the peptide corresponding to the sequence P-CT29Trp)

Calc. m/z: [H+M]+=1684.7 (the peptide corresponding to the sequence P-CT29MeTrp)

### Example 6: Development of N-methylleucine-accepting ARS

### Preparation of Wild-Type and Mutant LeuRS Proteins

Expression vectors having a polyhistidine sequence at the N-terminus and containing mutations listed in Table 9 were constructed. Subsequently, an expression strain was transformed with the vectors, and the mutant proteins of interest were purified with a nickel column from supernatants obtained by disrupting cells.

TABLE 9

| Name | Position 43 | Position 80 | Position 252 |
|---|---|---|---|
| LeuRS01(wt) | Y | D | T |
| LeuRS02 | G | D | T |
| LeuRS03 | A | D | T |
| LeuRS04 | E | D | T |
| LeuRS06 | F | D | T |
| LeuRS07 | Y | G | T |
| LeuRS08 | Y | A | T |
| LeuRS09 | E | G | T |
| LeuRS10 | E | A | T |
| LeuRS11 | D | G | T |
| LeuRS12 | D | A | T |
| LeuRS13 | A | G | T |
| LeuRS14 | G | G | T |
| LeuRS15 | Y | D | A |
| LeuRS16 | A | G | A |
| LeuRS17 | G | G | A |
| LeuRS18 | G | D | A |
| LeuRS19 | Y | G | A |
| LeuRS21 | E | G | A |
| LeuRS22 | E | A | A |
| LeuRS23 | D | G | A |

### Translational Introduction of N-methylleucine Using Wild-Type and Mutant LeuRSs

A template mRNA(R-L (SEQ ID NO: 201)) was synthesized from a template DNA (D-L (SEQ ID NO: 200)) by in vitro transcription reaction using RiboMAX Large Scale RNA production System T7 (Promega, P1300) and purified using RNeasy Mini kit (Qiagen).

D-L (CT23)
DNA sequence:
(SEQ ID NO: 200)
GGCGTAATACGACTCACTATAGGGTTAACTTTAACAAGGAGAAAAACATG
CGTCTCCGTGACTACAAGGACGACGACGACAAGTAAGCTTCG
R-L
RNA sequence:
(SEQ ID NO: 201)
GGGUUAACUUUAACAAGGAGAAAAACAUGCGUCUCCGUGACUACAAGGAC
GACGACGACAAGUAAGCUUCG

### Cell-Free Translation System

In order to confirm translational introduction of N-methylleucine, a desired polypeptide containing N-methylleucine was ribosomally synthesized by adding N-methylleucine and a mutant LeuRS to a cell-free translation system. The translation system used was PURE system, a reconstituted cell-free protein synthesis system from E. coli. Specifically, wild-type or mutant LeuRS and N-methylleucine were added to a solution containing a basic cell-free translation solution (1 mM GTP, 1 mM ATP, 20 mM creatine phosphate, 50 mM HEPES-KOH pH 7.6, 100 mM potassium acetate, 9 mM magnesium acetate, 2 mM spermidine, 1 mM dithiothreitol, 1.5 mg/ml E. coli MRE600 (RNase negative)-derived tRNA (Roche), 0.1 mM 10-HCO—H4 folate, 4 μg/ml creatine kinase, 3 μg/ml myokinase, 2 unit/ml inorganic pyrophosphatase, 1.1 μg/ml nucleoside diphosphate kinase, 0.6 μM methionyl-tRNA transformylase, 0.26 μM EF-G, 0.24 μM RF2, 0.17 μM RF3, 0.5 μM RRF, 2.7 μM IF1, 0.4 μM IF2, 1.5 μM IF3, 40 μM EF-Tu, 84 μM EF-Ts, 1.2 μM ribosome, 0.03 μM ArgRS, 0.13 μM AspRS, 0.11 μM LysRS, 0.03 μM MetRS, 0.02 μM TyrRS (wherein the proteins prepared by the inventors were essentially prepared as His-tagged proteins)), and 1 μM template mRNA, each 250 μM arginine, aspartic acid, lysine, methionine, and tyrosine, and left at 37° C. for 1 hour to ribosomally synthesize the peptide.

### Detection by Mass Spectroscopy

Mass spectroscopy was performed using MALDI-TOF MS for detecting peptides into which N-methylleucine is ribosomally introduced. Specifically, a solution containing 1 μM template mRNA (R-L (SEQ ID NO: 201)) and arginine, lysine, methionine, tyrosine, and aspartic acid (each final concentration 250 μM) added to the cell-free translation system as described above was prepared. LeuRS (final concentration 0.4-2 μM) and N-methylleucine (final concentration 5 mM) were added to the solution and incubated at 37° C. for 60 minutes. The resulting translation reaction products were purified with SPE C-TIP (Nikkyo Technos Co., Ltd) and analyzed with MALDI-TOF MS. Translation products were identified with MALDI-TOF MS spectrometry using a-cyano-4-hydroxycinnamic acid as a matrix.

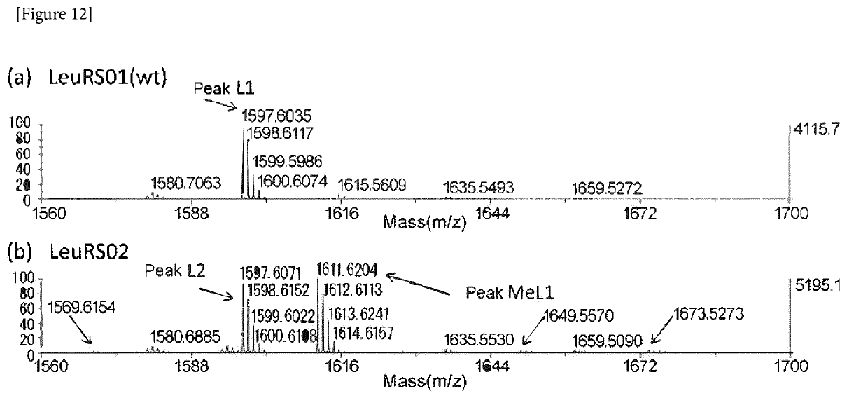

As a result of the translation using wild-type LeuRS (SEQ ID NO: 189), a peak corresponding to the target peptide peak P-CT23MeLeu containing N-methylleucine was not observed, but the peptide P-CT23Leu derived from Leu contaminated in the translation system in trace amounts (FIG. 12(a), PeakL1) was observed as an almost single product. On the other hand, when experiments were performed using the modified LeuRS 02 (SEQ ID NO: 187) with mutations introduced, the peak derived from P-CT29MeLeu (Peak MeL1, FIG. 12(b)) was observed, and the intensity of the peak was as strong as the intensity of the peak of the peptide P-CT23Leu containing Leu (Peak L2, FIG. 12(b)). In other words, it is demonstrated that the MeLeu-introduced peptide synthesized using the modified LeuRS 02 was more than that using wild-type LeuRS, suggesting that this modified LeuRS is an ARS that can introduce MeLeu into peptides more efficiently compared to wild-type LeuRS.

Peptide sequence P-CT23Leu
(SEQ ID NO: 202)
formylMetArgLeuArgAspTyrLysAspAspAspAspLys
Peptide sequence P-CT23MeLeu
(SEQ ID NO: 203)
formylMetArg[MeLeu]ArgAspTyrLysAspAspAspAspLys

MALDI-TOF MS:

Calc. m/z: [H+M]+=1597.7 (the peptide corresponding to the sequence P-CT23Leu)

Calc. m/z: [H+M]+=1611.7 (the peptide corresponding to the sequence P-CT23MeLeu)

### Example 7: Development of Modified ValRSs Having Increased Selectivity to N-methylvaline Achieved by Enhancing Valine-Hydrolyzing Ability in the Editing Domain

### Preparation of Wild-Type and Mutant ValRS Proteins

Expression vectors for modified ValRSs that have a polyhistidine sequence at the N-terminus and containing mutations (N43G,T45S) in the catalytic domain and mutation T279A(G) in the editing domain, which mutations increase activity to N-methylvaline, were constructed (Table 10). Then, an expression strain was transformed with the vectors, and the mutant proteins of interest were purified using a nickel column from supernatants obtained by disrupting cells.

TABLE 10

| | Catalytic domain | | Editing domain |
|---|---|---|---|
| Name | Position 43 | Position 45 | Position 279 |
| ValRS01(wt) | N | T | T |
| ValRS13-11 | G | S | T |
| ValRS66 | G | S | A |
| ValRS67 | G | S | G |

Aminoacylation Reaction with Valine and N-methylvaline Using Mutant ValRSs
Synthesis of E. coli tRNAVal by In Vitro Transcription Reaction

E. coli tRNA (R-tRNAVal1 (SEQ ID NO: 205)) was synthesized from a template DNA (D-tRNAVal1 (SEQ ID NO: 204)) by in vitro transcription reaction using RiboMAX Large Scale RNA production System T7 (Promega, P1300) in the presence of 7.5 mM GMP, and purified using RNeasy Mini kit (Qiagen).

D-tRNAVal1
tRNAVal1 DNA sequence:
(SEQ ID NO: 204)
GGCGTAATACGACTCACTATAGGGTGATTAGCTCAGCTGGGAGAGCACCT
CCCTTACAAGGAGGGGGTCGGCGGTTCGATCCCGTCATCACCCACCA
R-tRNAVal1
tRNAVal1 RNA sequence:
(SEQ ID NO: 205)
GGGUGAUUAGCUCAGCUGGGAGAGCACCUCCCUUACAAGGAGGGGGUCGG
CGGUUCGAUCCCGUCAUCACCCACCA

### Aminoacylation Reaction

For the aminoacylation reaction, the solution containing 50 μM transcribed tRNA, 10 mM HEPES-K (pH 7.6), and 10 mM KCl solution was heated at 95° C. for 2 minutes and then left at room temperature for 5 minutes or more to refold the tRNA. This tRNA solution was added to a final concentration of 10 μM to an acylation buffer (in final concentrations of 50 mM HEPES-K [pH 7.6], 2 mM ATP, 100 mM potassium acetate, 10 mM magnesium acetate, 1 mM DTT, 2 mM spermidine, 0.1 mg/mL Bovine Serum Albumin), mixed with a mutant ValRS (final concentration 2 μM) and N-methylvaline (final concentration 0.08 mM-5 mM) or valine (final concentration 0.031 mM-0.25 mM), and incubated at 37° C. for 10 minutes. Four volumes of a loading buffer (90 mM sodium acetate [pH 5.2], 10 mM EDTA, 95% (w/w) formamide, 0.1% (w/v) xylene cyanol) were added to the reaction solution and analyzed with acidic PAGE containing 6 M urea, and aminoacylation activity was detected by separating unreacted tRNA and aminoacyl-tRNA. RNA was stained with SYBR Gold (Life Technologies) and detected with LAS4000 (GE Healthcare).

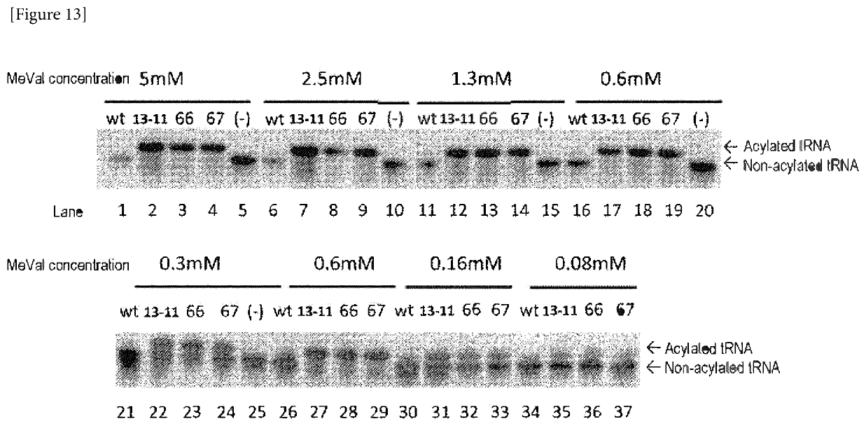

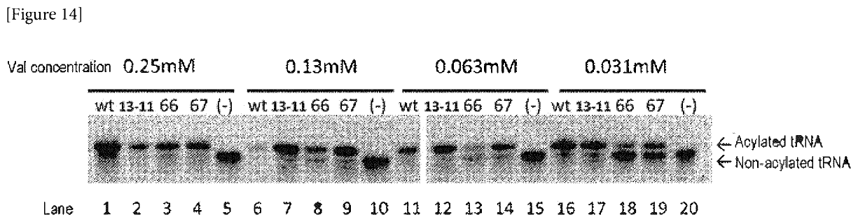

This result proved that the acylation abilities of the mutants 13-11, 66 (SEQ ID NO: 182), and 67 (SEQ ID NO: 183) were not very different at each N-methylvaline concentration when N-methylvaline was used as substrate (FIG. 13, for example, lanes 17-19). On the other hand, it was proved that when valine was used as a substrate, the acylation abilities of mutants 66 and 67 reduced compared to mutants 13-11 (FIG. 14, for example, lanes 17-19). This demonstrated that mutants 66 and 67 having mutations newly introduced into the editing domain are modified ValRSs that have reduced aminoacylation activity to valine and therefore has increased selectivity to N-methylvaline.

### INDUSTRIAL APPLICABILITY

The present invention provides modified aminoacyl-tRNA synthetases having increased reactivity with N-methyl amino acids compared to natural aminoacyl-tRNA synthetases (ARSs). The modified aminoacyl-tRNA synthetases according to the present invention can aminoacylate tRNAs with their corresponding N-methyl-substituted amino acids such as N-methyl-phenylalanine, N-methyl-valine, N-methyl-serine, N-methyl-threonine, N-methyl-tryptophan, and N-methyl-leucine more efficiently than natural aminoacyl-tRNA synthetases. The present invention can produce polypeptides containing N-methyl amino acids more efficiently.

1-15. (canceled)

16. A polypeptide comprising a modified phenylalanyl-tRNA synthetase (PheRS), wherein the PheRS is able to incorporate an N-methyl phenylalanine more efficiently than an original, natural PheRS.

17. A phenylalanyl-tRNA synthetase (PheRS) modified to enhance an aminoacylation reaction with an N-methyl phenylalanine, wherein the modification comprises at least one amino acid substitution.

18. The PheRS polypeptide according to claim 17 comprising
(a) a PheRS a, subunit modified at a position corresponding to glutamine at position 169 of PheRS a. subunit from Escherichia coli, or
(b) a PheRS α subunit having glycine or alanine at a position corresponding to glutamine at position 169 of PheRS u subunit from Escherichia coli.

19. The PheRS polypeptide according to claim 17, wherein the PheRS is derived from a bacterium.

20. The PheRS polypeptide according to claim 19, wherein the bacterium is Escherichia coli.

21. The PheRS polypeptide according to claim 17, wherein the PheRS polypeptide is selected from the group consisting of the following (a) and (b):
(a) a PheRS polypeptide comprising an amino acid sequence selected from the group consisting of SEQ ID NOs: 1 to 2, and
(b) a PheRS polypeptide comprising an amino acid sequence having at least 90% identity to an amino acid sequence selected from the group consisting of SEQ ID NOs: 1 to 2.

22. A polynucleotide encoding the PheRS polypeptide according to claim 17.

23. A vector comprising the polynucleotide according to claim 22.

24. A host cell comprising the PheRS polynucleotide according to claim 22.

25. A method for producing the PheRS polypeptide according to claim 17, comprising the step of culturing a host cell comprising a polypeptide encoding the PheRS polypeptide.

26. A method for producing a tRNA acylated with N-methyl phenylalanine, comprising the step of contacting the N-methyl phenylalanine with a tRNA in the presence of the PheRS polypeptide according to claim 17.

27. A method for producing a polypeptide comprising an N-methyl phenylalanine, comprising the step of performing translation in the presence of the PheRS polypeptide according to claim 17 and the N-methyl phenylalanine.

28. The method according to claim 27, wherein the step of performing translation is carried out in a cell-free translation system.

29. A fusion polypeptide comprising the PheRS polypeptide according to claim 17 and another polypeptide.

30. The fusion polypeptide according to claim 29, wherein the another polypeptide is a peptide tag.
